(19) United States
(12) Patent Application Publication
(10) Pub. No.: US 2019/0091673 A1
(43) Pub. Date: Mar. 28, 2019

(54) DISSOCIABLE NANOPARTICLES WITH INTER ALIA TRANSITION-METAL COMPLEX CATALYSTS

(71) Applicant: SELUX DIAGNOSTICS, INC., Watertown, MA (US)

(72) Inventors: Eric STERN, Watertown, MA (US); Aleksandar VACIC, Watertown, MA (US); Alec Nathanson FLYER, Watertown, MA (US); Benjamin SPEARS, Watertown, MA (US); Susan CLARDY, Watertown, MA (US)

(21) Appl. No.: 15/745,361

(22) PCT Filed: Jul. 15, 2016
(86) PCT No.: PCT/US2016/042589

§ 371 Date: Jan. 16, 2018

Related U.S. Application Data
(60) Provisional application No. 62/194,038, filed on Jul. 17, 2015.
(60) Provisional application No. 62/194,046, filed on Jul. 17, 2015.
(60) Provisional application No. 62/287,856, filed on Jan. 27, 2016.
(60) Provisional application No. 62/287,860, filed on Jan. 27, 2016.

(51) Int. Cl. B01J 31/16; B01J 31/18; B01J 35/00; C09K 11/07; G01N 33/58

(52) U.S. Cl. CPC B01J 31/1683; B01J 31/184; B01J 35/0013; C09K 2211/1018; G01N 33/587; B01J 2531/842; B01J 2231/005; C09K 11/07

(57) ABSTRACT

Nanoparticles for use in assay methods for detecting analytes in samples, which comprise a signal inducing agent, e.g. a transition-metal catalyst or a chemiluminophore, a chemiluminophore precursor, a soluble absorber, or a soluble absorber precursor. After binding to an analyte, the nanoparticle is dissociated by a chemical or physical trigger, e.g. an organic solvent or ultrasound, to release the signal inducing agent, which releases a detectable signal via a physical or chemical reaction. The nanoparticles comprising a chemiluminophore, a chemiluminophore precursor, a soluble absorber, or a soluble absorber precursor can also effect chemical reactions that serve as signal amplifiers.

### CROSS-REFERENCE TO RELATED APPLICATIONS

The present application claims the benefit under 35 U.S.C. § 119(e) of U.S. Provisional Patent Application Nos. 62/194,038, filed Jul. 17, 2015; 62/194,046, filed Jul. 17, 2015; 62/287,856, filed Jan. 27, 2016; and 62/287,860, filed Jan. 27, 2016, each of which is incorporated by reference in its entirety.

### BACKGROUND OF THE INVENTION

Many biochemical assays require labels for detection in order to convert a specific binding event into a measurable signal. In order to enhance the detection sensitivity of in vitro diagnostic assays, an amplification event is often performed. Labels may perform this amplification. For example, catalytic amplification may be performed by enzymes, such as horseradish peroxidase, alkaline phosphatase, etc., that are directly or indirectly bound to biological recognition molecules and thereby producing multiple detectable molecules, resulting in an amplification of each individual biochemical recognition event. Other reagents and methods for the development of assays that use non-enzymatic amplification can be useful (e.g., provide improved signal amplification and/or signal-to-noise ratios).

### SUMMARY OF THE INVENTION

Described herein are nanoparticles, and compositions and uses thereof, where the nanoparticle comprises a transition-metal catalyst, or an oxidized or reduced form thereof, that effects chemical reactions that serve as signal amplifiers.

In one aspect, the invention features a nanoparticle comprising
(a) a transition-metal catalyst; and
(b) one or more matrix-forming agents providing a dissociable matrix, wherein the transition-metal catalyst is embedded in the matrix.

In some embodiments, the matrix sequesters the transition-metal catalyst until said matrix is dissociated.

In some embodiments, the embedding of the transition-metal catalyst in the matrix is not primarily governed by electrostatic interactions.

In other embodiments, the transition-metal catalyst of (a) comprises a structure according to formula I,

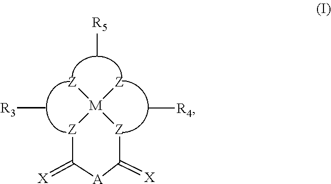

or an oxidized or reduced form thereof, wherein
M is a metal;
A is —CR₁R₂— or —NR_{1′}—;
   wherein when A is —CR₁R₂—, R₁ and R₂ are the same or different, linked or nonlinked, and each is selected from the group consisting of substituents which are unreactive, form strong bonds intramolecularly within said R₁ and R₂ and with the carbon C to which they are bound, are sterically hindered and are conformationally hindered such that oxidative degradation of a metal complex of the compound is restricted when the complex is in the presence of an oxidizing medium; and
   wherein when A is —NR_{1′}—, R_{1′} is C₁₋₂₀ alkyl, C₂₋₂₀ alkenyl, C₂₋₂₀ alkynyl, or phenyl;
   Z is a metal complexing atom selected from the group consisting of N, NH, and O;
   X is a functionality;
   wherein both Z and X are resistant to oxidative degradation such that each confers resistance to oxidative degradation to the metal complex of the compound when the complex is in the presence of an oxidizing medium;
   R₃ is a unit joining the adjacent Z atoms selected from the group consisting of:

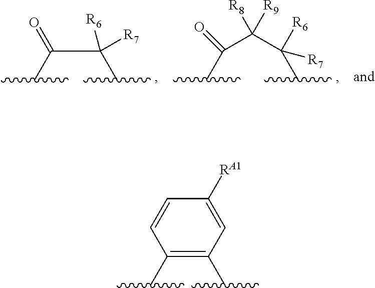

wherein R₆, R₇, R₈ and R₉ pairwise and cumulatively are the same or different and each is selected from the group consisting of hydrogen, C₁₋₂₀ alkyl, C₂₋₂₀ alkenyl, C₂₋₂₀ alkynyl, C₆₋₁₀ aryl, and halogen; or any pair of R₆, R₇, R₈ and R₉ can, together with the atoms to which they are attached, form a C₄₋₁₀ cycloalkyl;
R^{A1} is hydrogen, halogen, or —X¹—Y¹—Z¹, wherein
   X¹ is —C(R^{X1})₂—, —C(O)—, —C(O)O—, —C(O)NH—, —CR^{X1}═CR^{X1}—, —NR^{X1}—, —NR^{X1}C(O)—, —O—, or —OC(O)—, wherein R^{X1} is hydrogen or C₁₋₆ alkyl;
   Y¹ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y¹ are optionally and independently replaced by -Cy¹-,
—NR^{Y1}—, —N(R^{Y1})C(O)—, —C(O)N(R^{Y1})—, —O—, —C(O)—, —OC(O)—, —C(O)O—, or —N═N—, wherein R^{Y1} is hydrogen or C₁₋₆ alkyl; and
   each Cy¹ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
   Z¹ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
R₄ is a unit joining the adjacent Z atoms comprised of

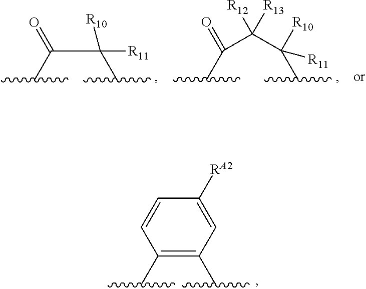

wherein R₁₀, R₁₁, R₁₂ and R₁₃ pairwise and cumulatively are the same or different and each is selected from the group consisting of hydrogen, C₁₋₂₀ alkyl, C₂₋₂₀ alkenyl, C₂₋₂₀ alkynyl, C₆₋₁₀ aryl, and halogen; or any pair of R₁₀, R₁₁, R₁₂ and R₁₃ can, together with the atoms to which they are attached, form a C₄₋₁₀ cycloalkyl;
   R^{A2} is hydrogen, halogen, or —X²—Y²—Z², wherein
      X² is —C(R^{X2})₂—, —C(O)—, —C(O)O—, —C(O)NH—, —CR^{X2}═CR^{X2}—, —NR^{X2}, —NR^{X2}C(O)—, —O—, or —OC(O)—, wherein R^{X2} is hydrogen or C₁₋₆ alkyl;
      Y² is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y² are optionally and independently replaced by -Cy²-,
   —NR^{Y2}—, —N(R^{Y2})C(O)—, —C(O)N(R^{Y2})—, —O—, —C(O)—, —OC(O)—, —C(O)O—, or —N═N—, wherein R^{Y2} is hydrogen or C₁₋₆ alkyl; and
      each Cy² is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
      Z² is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl
R₅ is a unit joining adjacent Z atoms selected from the group consisting of
   (i)

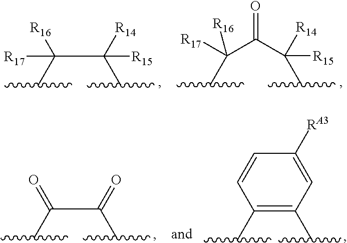

wherein R₁₄, R₁₅, R₁₆ and R₁₇ are the same or different and each is hydrogen, C₁₋₂₀ alkyl, C₂₋₂₀ alkenyl, C₂₋₂₀ alkynyl, C₆₋₁₀ aryl, and halogen;
or any pair of R₁₄, R₁₅, R₁₆ and R₁₇ can, together with the atoms to which they are attached, form a C₄₋₁₀ cycloalkyl;
R^{A3} is hydrogen, halogen, or —X³—Y³—Z³, wherein
   X³ is —C(R^{X3})₂—, —C(O)—, —C(O)O—, —C(O)NH—, —CR^{X3}═CR^{X3}—, —NR^{X3}—, —NR^{X3}C(O)—, —O—, or —OC(O)—, wherein R^{X3} is hydrogen or C₁₋₆ alkyl;
   Y³ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y³ are optionally and independently replaced by -Cy³-,
—NR^{Y3}—, —N(R^{Y3})C(O)—, —C(O)N(R^{Y3})—, —O—, —C(O)—, —OC(O)—, —C(O)O—, or —N═N—, wherein R^{Y3} is hydrogen or C₁₋₆ alkyl; and
   each Cy³ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
   Z³ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and

optionally a counter ion selected from H₂O, ammonium, and halogen.

In some embodiments, each Z is N.

In some embodiments, each X is independently O or S. In some embodiments, each X is O.

In certain embodiments, A is —CR₁R₂.

In other embodiments, A is —NR_{1′}—. In further embodiments, R_{1′} is C₁₋₂₀ alkyl (e.g., C₁₋₁₈ alkyl or C₁₋₁₂ alkyl).

In other embodiments, each of R₁ and R₂ is selected, independently, from the group consisting of hydrogen, halogen, and C₁₋₂₀ alkyl. In still other embodiments, R₁ and R₂ link to form a C₃₋₁₀ cycloaliphatic group.

In some embodiments, R₁ is C₁₋₂₀ alkyl (e.g., C₁₋₁₈ alkyl or C₁₋₁₂ alkyl). In some embodiments, R₂ is C₁₋₂₀ alkyl (e.g., C₁₋₁₈ alkyl or C₁₋₁₂ alkyl). In other embodiments, R₁ and R₂ link to form a C₃₋₁₀ cycloaliphatic group.

In certain embodiments, R₃ is a unit joining the adjacent Z atoms comprised of

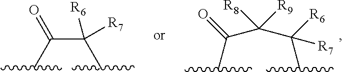

wherein each of R₆, R₇, R₈ and R₉ is, independently halogen, C₁₋₂₀ alkyl, C₂₋₂₀ alkenyl, or C₂₋₂₀ alkynyl. In other embodiments, R₆ and R₇, or R₈ and R₉, link to form a C₃₋₁₀ cycloaliphatic group.

In some embodiments, R₄ is a unit joining the adjacent Z atoms comprised of

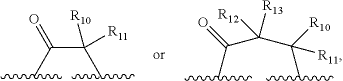

wherein each of R₁₀, R₁₁, R₁₂ and R₁₃ is, independently, halogen, C₁₋₂₀ alkyl, C₂₋₂₀ alkenyl, or C₂₋₂₀ alkynyl. In other embodiments, R₁₀ and R₁₁, or R₁₂ and R₁₃, link to form a C₃₋₁₀ cycloaliphatic group.

In certain embodiments, R₅ is a unit joining adjacent Z atoms selected from the group consisting of

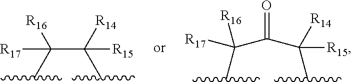

wherein each of R₁₄, R₁₅, R₁₆ and R₁₇ is independently selected from C₁₋₂₀ alkyl, C₆₋₁₀ aryl, and halogen. In other embodiments, R₁₄ and R₁₅, or R₁₆ and R₁₇, link to form a C₃₋₁₀ cycloaliphatic group.

In other embodiments, R₅ is an optionally-substituted aryl or heteroaryl group.

In some embodiments, any one of R₃, R₄, and R₅ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In another aspect, the invention features a nanoparticle comprising
(a) a transition-metal catalyst; and
(b) one or more matrix-forming agents providing a dissociable matrix, wherein the transition-metal catalyst is embedded in the matrix.

In some embodiments, the matrix sequesters the transition-metal catalyst until said matrix is dissociated.

In some embodiments, the embedding of the catalyst in the matrix is not primarily governed by electrostatic interactions.

In other embodiments, the transition-metal catalyst of (a) comprises a structure according to formula II,

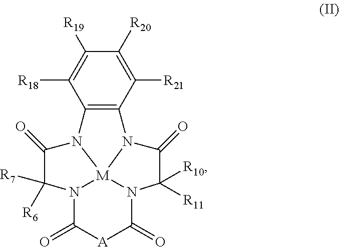

or an oxidized or reduced form thereof, wherein
   M is a metal;
A is —CR₁R₂— or —NR_{1′}—;
   wherein when A is —CR₁R₂—, each of R₁ and R₂ is, independently, hydrogen, C₁₋₂₀ alkyl, C₂₋₂₀ alkenyl, C₂₋₂₀ alkynyl, C₆₋₁₄ aryl, or halogen, or R₁ and R₂ may form, together with the carbon atom to which both are bound, a 3-10 membered ring; and
   wherein when A is —NR_{1′}—, R_{1′} is C₁₋₂₀ alkyl, C₂₋₂₀ alkenyl, C₂₋₂₀ alkynyl, or phenyl;
   each of R₆, R₇, R₁₀, and R₁₁ is, independently, hydrogen, C₁₋₂₀ alkyl, C₂₋₂₀ alkenyl, C₂₋₂₀ alkynyl, C₆₋₁₄ aryl, or halogen, or R₁ and R₂, or R₃ and R₄, or R₅ and R₆ may form, together with the carbon atom to which both are bound, a 3-10 membered ring; and
   each of is R₁₈, R₁₉, R₂₀, and R₂₁ is, independently, halogen, hydrogen, C₁₋₂₀ alkyl, C₂₋₂₀ alkenyl, C₂₋₂₀ alkynyl, C₆₋₁₄ aryl, amino, nitro, azido, cyano, —OH, C₁₋₂₀ alkoxy, —SH, C₁₋₂₀ thioalkoxy, C₆₋₁₄ aryloxy, —CO₂H, a carboxylic ester, an N-hydrosuccinimide ester group, an isothiocyanate group, an isocyanide group, or a 5-10-membered heterocyclic group.

In other embodiments, the transition-metal catalyst of (a) has a structure according to formula IIA.

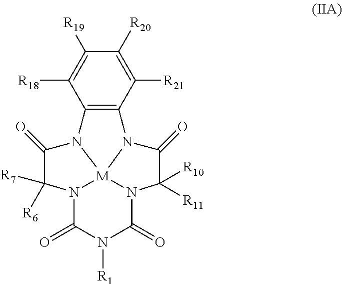

or an oxidized or reduced form thereof.

In still other embodiments, the transition-metal catalyst of (a) has a structure according to formula IIB,

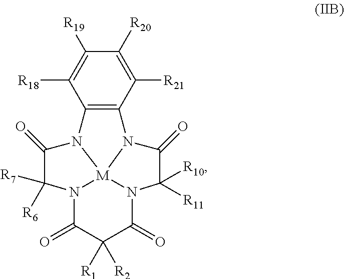

or an oxidized or reduced form thereof.

In certain embodiments, each of R₁ and R₂ is selected, independently, from the group consisting of hydrogen, halogen, and C₁₋₂₀ alkyl. In some embodiments, R₁ and R₂ link to form a C₃₋₁₀ cycloaliphatic group.

In other embodiments, one or more of R₆, R₇, R₁₀, and R₁₁ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In still other embodiments, one or more of R₁₈, R₁₉, R₂₀, and R₂₁ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In some embodiments, the nanoparticle comprises a transition metal catalyst having a structure that is

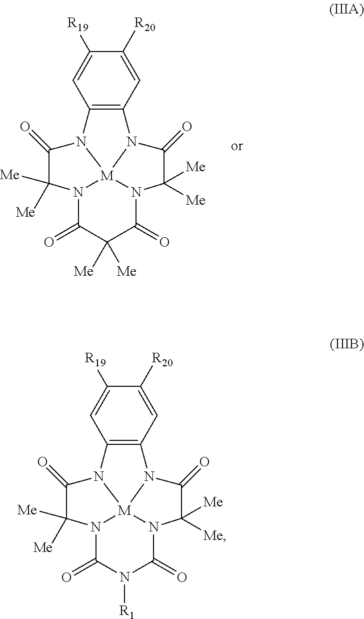

or an oxidized or reduced form thereof.

In certain embodiments, one or both of R₁₉ and R₂₀ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In some embodiments, the transition metal catalyst has a structure according to formula (IIIB) and R¹ optionally comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In other embodiments, the nanoparticle comprises a transition-metal catalyst having a structure that is,

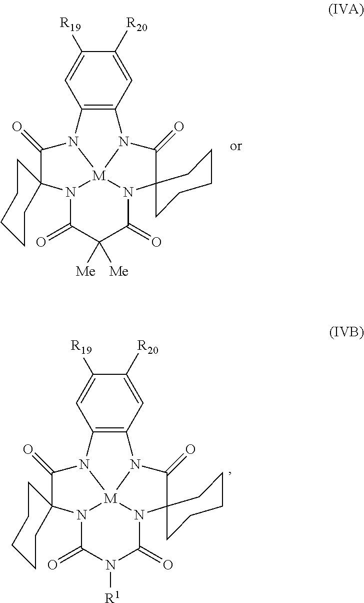

or an oxidized or reduced form thereof.

In some embodiments, one or both of R₁₉ and R₂₀ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In other embodiments, one or both of R₁₉ and R₂₀ is a norbornene or cyclooctene.

In still other embodiments, the transition metal catalyst has a structure according to formula (IVB) and R¹ optionally comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In some embodiments, M is a group 6, 7, 8, 9, 10, or 11 metal.

In other embodiments, M is Cr, Mn, Fe, Co, Ni, or Cu.

In embodiments, M is Fe (e.g., Fe(II) or Fe(III)).

In another aspect, the invention features a nanoparticle comprising:
(a) a transition-metal catalyst; and
(b) one or more matrix-forming agents providing a dissociable matrix, wherein the transition-metal catalyst is embedded in the matrix.

In some embodiments, the matrix sequesters the transition-metal catalyst until said matrix is dissociated.

In some embodiments, the embedding of the catalyst in the matrix is not primarily governed by electrostatic interactions.

In other embodiments, the transition-metal catalyst of (a) comprises a structure according to formula V,

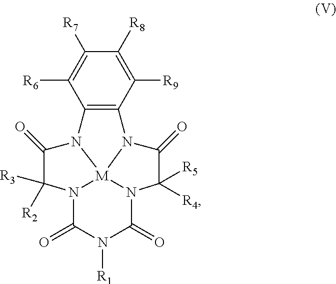

or an oxidized or reduced form thereof, wherein
M is a metal selected from the group consisting of Cr, Mn, Fe, Cu, Ni and Co;
R₁ is C₁₋₂₀ alkyl, C₂₋₂₀ alkenyl, C₂₋₂₀ alkynyl, or phenyl;
each of R₂, R₃, R₄, and R₅ is, independently, hydrogen, C₁₋₂₀ alkyl, C₂₋₂₀ alkenyl, C₂₋₂₀ alkynyl, or phenyl, or R₂ and R₃, or R₄ and R₅, combine to form a C₃₋₁₀ cycloaliphatic;
each of R₆, R₇, R₈, and R₉ is, independently, amino, nitro, azido, cyano, hydrogen, halogen, —NO₂, —COOH, —COOR₁₀, —COCl, —CN, C₁₋₂₀ alkyl, C₂₋₂₀ alkenyl, or C₂₋₂₀ alkynyl, wherein at least one of R₆, R₇, R₈, and R₉ is halogen, —NO₂, —COOH,
   —COOR₁₀, —COCl, or —CN:
R₁₀ is C₁₋₂₀ alkyl, C₂₋₂₀ alkenyl, C₂₋₂₀ alkynyl, phenyl, or 5-to-10-membered heterocyclyl.

In some embodiments, one or both of R₇ and R₈ is halogen, —NO₂, —COOH, —COOR₁₀, —COCl, —CN, or a N-hydroxysuccinimide ester group.

In certain embodiments, R₁, R₇, and/or R₈ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In still other embodiments, one or more of R₂, R₃, R₄, and R₅ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

In some embodiments, each of R₂, R₃, R₄, and R₅ is C₁ alkyl.

In some embodiments, the transition-metal catalyst further comprises a neutral ligand. In some embodiments, the neutral ligand is H₂O, NH₃, CO, or NO.

In some embodiments, the transition-metal catalyst further comprises a counterion. In some embodiments, the counterion is negatively charged (i.e., an anion). In other embodiments, the counterion is positively charged (i.e., a cation).

In embodiments, M is Fe (e.g., Fe(II) or Fe(III)).

In certain embodiments, M is Fe(III). In embodiments, the transition-metal catalyst further comprises a counterion having a charge of +1. In some embodiments, the counterion is a cationic surfactant (e.g., Adogen 464).

In some embodiments, the transition-metal catalyst mediates an oxidative or reductive transformation on a compound. In other embodiments, the transition-metal catalyst mediates an oxidative reaction on a compound (e.g., the substrate can act as a detector of a reactive oxygen species (ROS)). Exemplary substrates include those provided in Gomes et al., J. Biochem. Biophys. Methods 65, 45-80, 2005 (see, e.g., Table 1 on pages 48-49), or derivatives thereof. For example, in some embodiments, the substrate is selected from: hydroethidine (HE); 1,3-diphenylisobenzofuran (DPBF), 2-(2-pyridyl)-benzothioazoline; 2,7-dichlorodihydrofluorescein (DCFH); 7-hydroxy-6-methoxy coumarin (scopoletin); N-acetyl-3,7-dihydroxyphenoxazine (Amplex Red); 4-hydroxy-3-methoxy-phenylacetic acid (HVA or homovanillic acid); dihydrorhodamine 123 (DHR); 4-(9-anthroyloxy)-2,2,6,6,-tetramethylpiperidine-1-oxyl; 1,3-cyclohexanedione (CHD); sodium terephthalate; coumarin-3-carboxylic acid (3-CCA); N-succinimidyl ester of coumarin-3-carboxylic acid (SECCA); 2-[6-(4′-hydroxy)phenoxy-3H-xanthen-3-on-9-yl]benzoic acid (HPF); 2-[6-(4′-amino)phenoxy-3H-xanthen-3-on-9-yl]benzoic acid (APF); cis-parinaric acid (cis-PnA, (18:14):9,11,13,15-cis-trans-trans-cis-octadecaenoic acid); 4,4-difluoro-5-(4-phenyl-1,3-butadienyl)-4-bora-3a,4a-diaza-s-indacene-3-undecanoic acid (C₁₁—BODIPY); lipophilic fluorescein derivatives; dipyridamole; diphenyl-1-pyrenylphosphine (DPPP); 2,7-dichlorodihydrofluorescein acetate (DCFH-DA); beta-physcoerythrin; fluorescein; and 6-carboxyfluorescein, or a derivative thereof.

In another aspect, the invention features a nanoparticle comprising
(a) a transition-metal catalyst having the structure M(X)ₙ(R)ₒ, wherein
   M is a transition-metal;
   n is 0, 1, 2, 3, or 4;
   o is 2, 3, 4, 5, or 6;
   X is an ion of a Group V, VI, or VII element;
   R is a ligand selected from monodentate phosphine ligands, bidentate phosphine ligands, monodentate Schiff base ligands, bidentate Schiff base ligands, tridentate Schiff base ligands, macrocyclic ligands, pentamethylcyclopentadiene, monodentate arsine, or N-heterocyclic carbene ligands; and
(b) one or more matrix-forming agents providing a dissociable matrix, wherein the transition-metal catalyst is embedded in the matrix;
where the transition-metal catalyst of (a) catalyzes a bond formation reaction or a bond cleavage reaction that modulates the fluorescent or chromogenic properties of a substrate compound.

In some embodiments, X is a halogen (e.g., F, Cl, Br, or I). In other embodiments, X is an amino ligand. In still other embodiments, X is an oxygen ligand (e.g., hydroxyl, alkoxyl, or phenoxyl). In some embodiments, X is a phosphorus-containing ligand (e.g., a monodentate or polydentate phosphine ligand). In still other embodiments, X is a sulfur-containing ligand (e.g., thiol, thioalkoxyl, or thiophenoxyl). In some embodiments, X is a monodentate ligand. In other embodiments, two or more X (e.g., 2 or 3) combine to form a polydentate ligand.

In some embodiments, R is selected from: triarylphosphines, trialkylphosphines, aryldialkylphosphines, 1,1′-bis(diphenylphosphino)ferrocene, tricycloalkylphosphine, (1,1′-biphenyl-2-yl)dicyclohexylphosphine, aryldicycloalkylphosphines, 2,6-bis[1-(phenyl)iminoethyl]pyridine, 3-[[3-[(E)-[[2,6-bis(1-methylethyl)phenyl]imino]methyl]-4-hydroxyphenyl]methyl]-1-methyl-imidazolium chloride, 3,7,11,17-tetraazabicyclo[11.3.1]heptadeca-1(17),13,15-triene, tetrasulfophthalocyanine, pentamethylcyclopentadiene, triarylarsines, 1,3-diisopropylimidazolium tetrafluoroborate, 1,3-bis(1-adamantanyl)imidazolium tetrafluoroborate, 1,3-bis-(tert-butyl)-4,5-dihydro-1H-imidazolium tetrafluoroborate, N,N′-(2,4,6-trimethyl)dihydroimidazolium chloride, and N,N′-(2,6-diisopropylphenyl)dihydroimidazolium chloride.

In some embodiments, the embedding of the catalyst in the matrix is not primarily governed by electrostatic interactions.

In other embodiments, the matrix sequesters the transition-metal catalyst until said matrix is dissociated.

In still other embodiments, the substrate compound comprises a functional group that quenches fluorescence when covalently bound to the substrate compound.

In some embodiments, transition-metal catalyst induces fluorescence by mediating a bond cleavage reaction of the fluorescence quenching functional group in the substrate compound.

In still other embodiments, the substrate compound is a halogenated boron dipyrromethane (BODIPY) compound.

In certain embodiments, the substrate compound has a structure that is

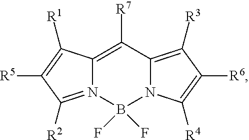

wherein
each of R¹, R², R³, R⁴, R⁵, and R⁶ is, independently, H, halogen, CN, C₁₋₂₀ alkyl, C₂₋₂₀ alkenyl, C₂₋₂₀ alkyl, C₁₋₂₀ alkoxy, —O(CH₂CH₂O)ₙCH₃, or —OCH((CH₂CH₂O)ₙCH₃)₂,
R⁷ is H, halogen, CN, C₁₋₂₀ alkyl, C₂₋₂₀ alkenyl, C₂₋₂₀ alkyl, C₁₋₂₀ alkoxy, C₆₋₁₀ aryl, or 5-to-10-membered heteroaryl;
each n is, independently, an integer between 1-6; and
wherein at least one of R¹, R², R³, R⁴, R⁵, R⁶, and R⁷ is halogen.

In some embodiments, each of R¹, R², R³, and R⁴ is, independently, H or C₁₋₂₀ alkyl.

In still other embodiments, at least one of R¹, R², R³, and R⁴ comprises a carboxylic acid substituent.

In certain embodiments, one or both of R⁵ and R⁶ is halogen.

In other embodiments, one or both of R⁵ and R⁶ is bromo or iodo.

In some embodiments, the substrate compound has the following structure,

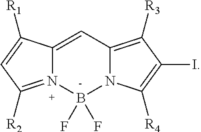

In still other embodiments, R⁷ is H or phenyl.

In some embodiments, M is Pd(O), Pd(II), Rh(I), Rh(III), Ir(I), Ir(III), Ru(II), Ru(III), Pt(O), Pt(II), or Cu(II).

In certain embodiments, the transition-metal catalyst comprises monodentate phosphine ligands, bidentate phosphine ligands, monodentate Schiff base ligands, bidentate Schiff base ligands, tridentate Schiff base ligands, macrocyclic ligands, pentamethylcyclopentadiene, monodentate arsine, or N-heterocyclic carbene ligands.

In other embodiments, the transition-metal catalyst comprises a ligand selected from: triarylphosphines, trialkylphosphines, aryldialkylphosphines, 1,1′-bis(diphenylphosphino)ferrocene, tricycloalkylphosphine, (1,1′-biphenyl-2-yl)dicyclohexylphosphine, aryldicycloalkylphosphines, 2,6-bis[1-(phenyl)iminoethyl]pyridine, 3-[[3-[(E)-[[2,6-bis(1-methylethyl)phenyl]imino]methyl]-4-hydroxyphenyl]methyl]-1-methyl-imidazolium chloride, 3,7,11,17-tetraazabicyclo[11.3.1]heptadeca-1(17),13,15-triene, tetrasulfophthalocyanine, pentamethylcyclopentadiene, triarylarsines, 1,3-diisopropylimidazolium tetrafluoroborate, 1,3-bis(1-adamantanyl)imidazolium tetrafluoroborate, 1,3-bis-(tert-butyl)-4,5-dihydro-1H-imidazolium tetrafluoroborate, N,N′-(2,4,6-trimethyl)dihydroimidazolium chloride, and N,N′-(2,6-diisopropylphenyl)dihydroimidazolium chloride.

In certain embodiments, M is Pd(II) or Pd(O).

In other embodiments, the nanoparticle comprises Pd(PCy₃)₂Cl₂, Pd(PPh₃)₂Cl₂, Pd(PPh₃)₄, Pd₂(dba)₃, Pd(TFA)₂, Pd(MeCN)₂Cl₂, Pd(acac)₂, Pd(amphos)Cl₂, Pd(dppf)Cl₂, Pd(dtbpf)Cl₂, Na₂PdCl₄, PdC, (NH₄)₂PdCl₄, PdBr₂, Pd(OAc)₂, or tris(dibenzylideneacetone)dipalladium(0).

In some embodiments, the matrix-forming agent comprises an organic polymer.

In certain embodiments, the matrix-forming agent is a non-degradable polymer (e.g., polystyrene, novolac, poly vinyl acetate, poly methyl methacrylate, poly vinyl pyrrole, poly vinyl acetate, polyisoprene, or polybutadiene).

In other embodiments, the matrix-forming agent is a polymer (e.g., a co-polymer) containing a hydrolyzable functionality (e.g., a polymer such as PLGA, PLA, or poly-ε-caprolactone). In other embodiments, the polymer is Examples of biodegradable polymers include, but are not limited to, poly(lactide), poly(glycolide), poly(orthoesters), poly(caprolactones), polylysine, poly(ethylene imine), poly(acrylic acid), poly(urethanes), poly(anhydrides), poly(esters), poly(trimethylene carbonate), poly(ethyleneimine), poly(acrylic acid), poly(urethane), poly(beta amino esters), or is a copolymer thereof (e.g., poly(lactide-co-glycolide) (PLGA)).

In still other embodiments, the nanoparticle comprises a matrix-forming agent that forms an inorganic matrix. In some embodiments, the inorganic matrix-forming agent comprises iron oxide, cerium oxide, ruthenium oxide, copper oxide, copper, gold, silver, titanium dioxide, silicon, silicon nitride, tin oxide, carbon nanotubes, vanadium oxide, alumina, aluminum, cobalt oxide, platinum, palladium, zinc oxide, magnesium oxide, manganese oxide, and/or nickel oxide.

In certain embodiments, the matrix comprises a covalent bond to the transition-metal catalyst.

In embodiments, a matrix comprising a covalent bond to a transition-metal catalyst is formed from Compound S1.6 as described herein.

In embodiments, a matrix comprising a covalent bond to a transition-metal catalyst is formed from a metalorganic compound comprising a transition metal (e.g., Fe) and Compound S1.6 as described herein.

In embodiments, a matrix comprising a covalent bond to a transition-metal catalyst is formed from Compound S2.6 as described herein.

In embodiments, a matrix comprising a covalent bond to a transition-metal catalyst is formed from a metalorganic compound comprising a transition metal (e.g., Fe) and Compound S2.6 as described herein.

In embodiments, a matrix comprising a covalent bond to a transition-metal catalyst is formed from Compound S3.7 as described herein.

In embodiments, a matrix comprising a covalent bond to a transition-metal catalyst is formed from a metalorganic compound comprising a transition metal (e.g., Fe) and Compound S3.7 as described herein.

In embodiments, a matrix comprising a covalent bond to a transition-metal catalyst is formed from Compound S3.11 as described herein.

In embodiments, a matrix comprising a covalent bond to a transition-metal catalyst is formed from Compound S4.2 as described herein.

In embodiments, a matrix comprising a covalent bond to a transition-metal catalyst is formed from a metalorganic compound comprising a transition metal (e.g., Fe) and Compound S4.2 as described herein.

In embodiments, a matrix comprising a covalent bond to a transition-metal catalyst is formed from Compound S5.3 as described herein.

In embodiments, a matrix comprising a covalent bond to a transition-metal catalyst is formed from a metalorganic compound comprising a transition metal (e.g., Fe) and Compound S5.3 as described herein.

In embodiments, a matrix comprising a covalent bond to a transition-metal catalyst is formed from Compound S6.3 as described herein.

In embodiments, a matrix comprising a covalent bond to a transition-metal catalyst is formed from a metalorganic compound comprising a transition metal (e.g., Fe) and Compound S6.3 as described herein.

In embodiments, a matrix comprising a covalent bond to a transition-metal catalyst is formed from any of the ligands described in Scheme 7 as described herein (e.g., Compound S7.1, Compound S7.2, Compound S7.4, or Compound S7.6).

In embodiments, a matrix comprising a covalent bond to a transition-metal catalyst is formed from a metalorganic compound comprising a transition metal (e.g., Fe) and any of the ligands described in Scheme 7 as described herein (e.g., Compound S7.1, Compound S7.2, Compound S7.4, or Compound S7.6).

In embodiments, a matrix comprising a covalent bond to a transition-metal catalyst is formed from Compound S7.5 or Compound S7.7.

In embodiments, a matrix comprising a covalent bond to a transition-metal catalyst is formed from any of the ligands described in Scheme 8 as described herein (e.g., Compound S8.1, Compound S8.2, Compound S8.4, or Compound S8.6).

In embodiments, a matrix comprising a covalent bond to a transition-metal catalyst is formed from a metalorganic compound comprising a transition metal (e.g., Fe) and any of the ligands described in Scheme 8 as described herein (e.g., Compound S8.1, Compound S8.2, Compound S8.4, or Compound S8.6).

In embodiments, a matrix comprising a covalent bond to a transition-metal catalyst is formed from Compound S8.5 or Compound S8.7.

In embodiments, a matrix comprising a covalent bond to a transition-metal catalyst is formed from Compound S8.9 or Compound S8.11 as described herein.

In embodiments, a matrix comprising a covalent bond to a transition-metal catalyst is formed from a metalorganic compound comprising a transition metal (e.g., Fe) and Compound S8.9 or Compound S8.11 as described herein.

In embodiments, a matrix comprising a covalent bond to a transition-metal catalyst is formed from Compound S8.10 or Compound S8.12 as described herein.

In embodiments, a matrix comprising a covalent bond to a transition-metal catalyst is formed from any of the ligands described in Scheme 9 as described herein (e.g., Compound S9.1, Compound S9.2, Compound S9.4, or Compound S9.6).

In embodiments, a matrix comprising a covalent bond to a transition-metal catalyst is formed from a metalorganic compound comprising a transition metal (e.g., Fe) and any of the ligands described in Scheme 9 as described herein (e.g., Compound S9.1, Compound S9.2, Compound S9.4, or Compound S9.6).

In embodiments, a matrix comprising a covalent bond to a transition-metal catalyst is formed from Compound S9.5 or Compound S9.7 as described herein.

In embodiments, a matrix comprising a covalent bond to a transition metal is formed from Compound S10.7 as described herein.

In embodiments, a matrix comprising a covalent bond to a transition-metal catalyst is formed from a metalorganic compound comprising a transition metal (e.g., Fe) and Compound S10.7 as described herein.

In embodiments, a matrix comprising a covalent bond to a transition metal is formed from Compound S10.8 as described herein.

In embodiments, a matrix comprising a covalent bond to a transition-metal catalyst is formed from any of the ligands described in Scheme 11 as described herein (e.g., Compound S11.1, Compound S11.2, Compound S11.4, or Compound S11.5).

In embodiments, a matrix comprising a covalent bond to a transition-metal catalyst is formed from a metalorganic compound comprising a transition metal (e.g., Fe) and any of the ligands described in Scheme 11 as described herein (e.g., Compound S11.1, Compound S11.2, Compound S11.4, or Compound S11.5).

In embodiments, a matrix comprising a covalent bond to a transition metal is formed from Compound S11.3 or Compound S11.6 as described herein.

In embodiments, a matrix comprising a covalent bond to a transition-metal catalyst is formed from Compound S12.8 as described herein.

In embodiments, a matrix comprising a covalent bond to a transition-metal catalyst is formed from a metalorganic compound comprising a transition metal (e.g., Fe) and Compound S12.8 as described herein.

In embodiments, a matrix comprising a covalent bond to a transition-metal catalyst is formed from Compound S12.9 as described herein.

In embodiments, a matrix comprising a covalent bond to a transition metal is as described in FIG. 1.

In certain embodiments, the matrix comprises a non-covalent interaction with the transition-metal catalyst.

In further embodiments, the non-covalent interaction with the transition-metal catalyst is a hydrophobic interaction, a hydrogen bonding interaction, or a van der Waals interaction.

In still other embodiments, the nanoparticle comprises an outer surface that comprises one or more functional groups for conjugating the nanoparticle to a binding agent.

In some embodiments, the nanoparticle further comprises an inner layer between the matrix core and the outer surface.

In still other embodiments, the binding agent comprises an antibody, ligand, protein, small molecule, aptamer, ss-DNA, ss-RNA, or ss-PNA.

In still other embodiments, the matrix comprises a further catalyst species.

In some embodiments, the matrix further comprises a compound that is a chemiluminophore, a chemiluminophore precursor, an absorber, or an absorber precursor.

In still other embodiments, the matrix comprises solvent dyes and/or water-soluble dyes.

In certain embodiments, the matrix comprises fluorescein dilaurate. fluorescein, rhodamine, rhodamine B octadecyl ester, Oregon green, eosin, Texas red, BODIPY, AlexaFluor, Atto, cyanine, indocarbocyanine, oxacarbocyanine, thiacarbocyanine, merocyanine, dansyl, prodan, coumarin, pyridyloxazole, nitrobenzoxadiazole, benzoxadiazole, anthraquinone, cascade blue, Nile red, Nile blue, cresyl violet, proflavin, acridine orange, acridine yellow, auramine, crystal violet, malachite green, porphin, phthalocyanine, bilirubin, 9,10-diphenylanthracene, 1-chloro-9,10-diphenylanthracene, 9,10-bis(phenylethynyl)anthracene, 1-chloro-9,10-bis(phenylethynyl)anthracene, 2-chloro-9,10-bis(phenylethynyl)anthracene, 1,8-dichloro-9,10-bis(phenylethynyl)anthracene, rubrene, 2,4-di-tert-butylphenyl-1,4,5,8-tetracarboxynaphthalene diamie, 5,12-bis(phenylethynyl)naphthacene, violanthrone, 16,16-(1,2-ethylenedioxy)violanthrone, 16,17-dihexyloxyviolanthrone, 16,17-butyloxyviolanthrone, N,N′-bis(2,5-di-tert-butylphenyl)-3,4,9,10-perylenedicarboximide, 1-N,N′-dibutylaminoanthracene, 6-methylacridinium iodide, or luminol, or a derivative thereof.

In some embodiments, the molar ratio of the compound: transition-metal catalyst that is about 10:1 to about 1:1, about 10:1 to about 3:1, about 8:1 to about 3:1, or about 5:1 to about 3:1.

In still other embodiments, the matrix further comprises a second transition-metal catalyst.

In another aspect, the invention feature a composition comprising any of the nanoparticles described herein, wherein said composition has a size distribution of nanoparticles between about 10 nm and less than about 10 μm, between about 10 nm to about 1 μm, about 10 nm to about 1 μm, about 10 nm to about 500 nm, about 10 nm to about 300 nm, or about 50 nm to about 300 nm.

In some embodiments, the composition has a size distribution of nanoparticles between about 25 nm and about 250 nm, about 25 nm and about 200 nm, about 25 nm and about 175 nm, about 25 nm and about 100 nm, or about 50 nm and about 100 nm.

In other embodiments, the composition has a polydispersity index of below about 0.35, below about 0.25, or below about 0.15.

In another aspect, the present invention is a nanoparticle comprising
(a) a transition-metal catalyst; and
(b) one or more matrix-forming agents providing a dissociable matrix, wherein the transition-metal catalyst is embedded in the matrix;
wherein
said transition-metal catalyst of (a) is selected from:

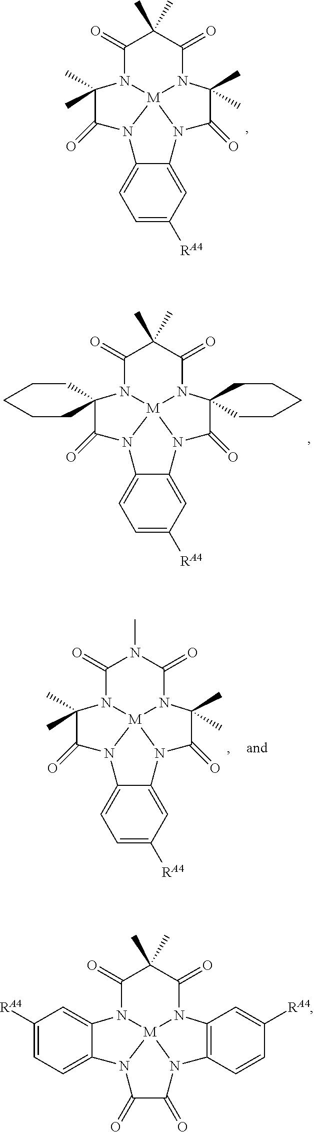

wherein
   M is a metal selected from Fe, Mg, Cu, Mn, Pd, Pt, Ag, Ru, and Ce; and
      R^{A4} is hydrogen, halogen, or —X⁴—Y⁴—Z⁴, wherein
         X⁴ is —C(R^{X4})₂—, —C(O)—, —C(O)O—, —C(O)NH—, —CR^{X4}═CR^{X4}—, —NR^{X4}—, —NR^{X4}C(O)—, —O—, or —OC(O)—, wherein R^{X4} is hydrogen or C₁₋₆ alkyl;
         Y⁴ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y⁴ are optionally and independently replaced by -Cy⁴-,
      —NR^{Y4}—, —N(R^{Y4})C(O)—, —C(O)N(R^{Y4})—, —O—, —C(O)—, —OC(O)—, —C(O)O—, or —N═N—, wherein R^{Y4} is hydrogen or C₁₋₆ alkyl; and
         each Cy⁴ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
   Z⁴ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
   a suitable counter ion selected from H₂O and halogen.

In embodiments, M is Fe (e.g., Fe(II) or Fe(III)).

In embodiments, R^{A4} is halogen (e.g., —F, —Cl, —Br, or —I).

In embodiments, R^{A4} is hydrogen.

In embodiments, R^{A4} is X⁴—Y⁴—Z⁴.

In one aspect, the invention features a polymer that includes a repeating unit including one or more signal-inducing agents (e.g., a repeating unit comprising a covalent attachment to any signal-inducing agent described herein).

In embodiments, a signal-inducing agent is releasable (e.g., a signal-inducing agent is released from a polymer via hydrolysis of one or more hydrolyzable groups in the polymer).

In embodiments, a polymer comprises multiple different signal-inducing agents.

In embodiments, a polymer comprises co-, alt-, branched-, or similar and/or hybrid structures.

In embodiments, a polymer includes a cleavable group that is within the backbone of the polymer.

In embodiments, a polymer includes a cleavable group that is pendant to the backbone of the polymer.

In embodiments, a polymer includes one or more non-payload elements for stability.

In embodiments, a polymer includes a covalent attachment to one or more detection species.

In embodiments, a polymer has a structure according to formula (A),

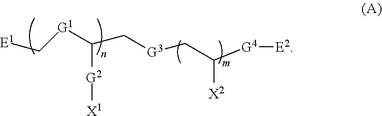

E¹ is independently hydrogen.
E² is independently hydrogen or a detection species.
Each of G¹, G², G³, and G⁴ is independently a covalent bond or cleavable group.
n is independently an integer of 1 to 100.
m is independently an integer of 0 to 100.
X¹ is a signal-inducing agent.
X² is hydrogen or non-payload element for stability.

In embodiments, each of G¹, G², G³, and G⁴ is independently a covalent bond.

In embodiments, one or more of G¹, G², G³, and G⁴ is independently a cleavable group.

In embodiments, X¹ is a signal-inducing agent comprising a transition metal catalyst (e.g., X¹ comprises any transition metal catalyst described herein).

In embodiments, E² is a detection species.

In embodiments, a polymer includes a repeating unit having a structure according to substructure S3.13,

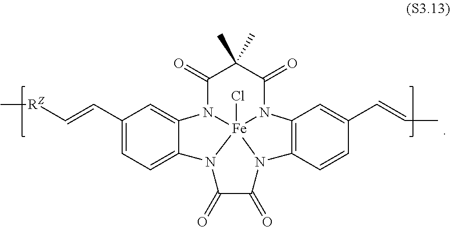

R^{Z} is hydrogen, halogen, or —X^{Z1}—Y^{Z1}—Z^{Z1}, wherein X^{Z1} is —C(R^{XZ1})₂—, —C(O)—, —C(O)O—, —C(O)NH—, —CR^{XZ1}═CR^{XZ1}—, —NR^{Z1}—, —NR^{XZ1}C(O)—, —O—, or —OC(O)—, wherein R^{XZ1} is hydrogen or C₁₋₆ alkyl; Y^{Z1} is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y^{Z1} are optionally and independently replaced by -Cy^{Z1}-, —NR^{YZ1}—, —N(R^{YZ1})C(O)—, —C(O)N(R^{YZ1})—, —O—, —C(O)—, —OC(O)—, —C(O)O—, or —N═N—, wherein R^{YZ1} is hydrogen or C₁₋₆ alkyl; and each Cy^{Z1} is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and Z^{Z1} is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl.

In one aspect, the invention features a nanoparticle that includes a polymeric matrix. In embodiments, a polymeric matrix includes a polymer that includes a repeating unit including one or more signal-inducing agents (e.g., any such polymer described herein).

In embodiments, a nanoparticle includes a compound having a structure according to S1.12 as described herein.

In embodiments, a nanoparticle includes a compound having a structure according to S2.12 as described herein. In embodiments, a nanoparticle includes a compound having a structure according to S3.13 as described herein.

In embodiments, a nanoparticle includes a compound having a structure according to S4.3 as described herein.

In embodiments, a nanoparticle includes a compound having a structure according to S5.4 as described herein.

In embodiments, a nanoparticle includes a compound having a structure according to S6.4 as described herein.

In one aspect, the invention features a nanoparticle comprising
(a) a compound that is chemiluminophore, a chemiluminophore precursor, a soluble absorber, or a soluble absorber precursor; and
(b) optionally one or more matrix-forming agents providing a matrix, wherein the compound of (a) is embedded in the matrix.

In some embodiments, the embedding of the compound of (a) is not primarily governed by electrostatic interactions.

In other embodiments, the embedding of the compound of (a) is primarily governed by surfactant stabilization during formation of the matrix.

In further embodiments, the matrix sequesters the compound of (a) until said matrix is dissociated.

In still other embodiments, the nanoparticle comprises at least about 20 mol % of the compound of (a).

In some embodiments, the compound of (a) is a fluorescein or rhodamine compound (e.g., a fluorescein or a rhodamine compound comprising acyl or sulfonyl functional groups that modulate the fluorescence of the compound).

In certain embodiments the compound of (a) is an acylated or alkylated fluorescein or an acylated or alkylated rhodamine.

In other embodiments, the compound of (a) is a fluorescein compound having a structure according to formula A,

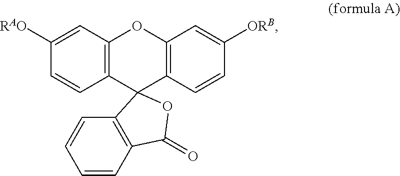

wherein each of R^{A} and R^{B} is, independently, acetyl, propionyl, butyryl, valeryl, hexanoyl, heptanoyl, decanoyl, dodecanoyl, hexadecanoyl, acrylyl, methanesulfonyl, isobutoxy carbonyl, furoyl, benzoyl, or —CH₂OC(═O)CH₃. In some embodiments, each of R^{A} and R^{B} is, independently, —C(O)(CH₂)ₓCH₃, where x is an integer between 0-20. In some embodiments, R^{A} and R^{B} are the same. In other embodiments, R^{A} and R^{B} are different. In still other embodiments, the compound of formula A further comprises 1, 2, or 3 substituent groups selected from halogen (e.g., F, Cl, Br, or I), C₁₋₆ alkyl, and C₁₋₆ alkoxy.

In other embodiments, the compound of (a) is a fluorescein compound having a structure according to formula B,

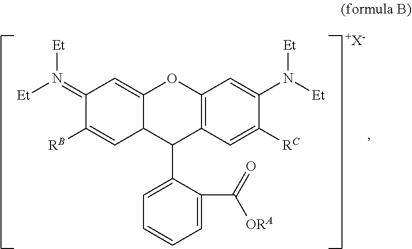

wherein R^{A} is a C₁₋₂₀ alkyl or a 5-10-membered heterocyclyl (e.g., an N-hydroxysuccinimide), and R^{B} and R^{C} are, independently, selected from hydrogen, halogen (e.g., F, Cl, Br, or I), C₁₋₆ alkyl, and C₁₋₆ alkoxy. In some embodiments, R^{B} and R^{C} are the same. In other embodiments, R^{B} and R^{C} are different.

In still other embodiments, the compound of (a) is fluorescein dilaurate, rhodamine B octadecyl ester, or rhodamine B hexyl ester.

In still other embodiments, the compound of (a) is a compound selected from: Oregon green, eosin, Texas red, BODIPY, AlexaFluor, Atto, cyanine, indocarbocyanine, oxacarbocyanine, thiacarbocyanine, merocyanine, dansyl, prodan, coumarin, 3-(2-benzothiazolyl)-7-(diethylamino)coumarin (“coumarin 6”), 3-(2-N-methylbenzimidazolyl)-7-N,N-diethylaminocoumarin (“coumarin 30”), 7-amino-4-(trifluoromethyl)coumarin (“coumarin 151”), pyridyloxazole, nitrobenzoxadiazole, benzoxadiazole, anthraquinone, cascade blue, Nile red, Nile blue, cresyl violet, proflavin, acridine orange, acridine yellow, auramine, crystal violet, malachite green, porphin, phthalocyanine, bilirubin, 9,10-diphenylanthracene, 1-chloro-9,10-diphenylanthracene, 9,10-bis(phenylethynyl)anthracene, 1-chloro-9,10-bis(phenylethynyl)anthracene, 2-chloro-9,10-bis(phenylethynyl)anthracene, 1,8-dichloro-9,10-bis(phenylethynyl)anthracene, rubrene, 2,4-di-tert-butylphenyl-1,4,5,8-tetracarboxynaphthalene diamie, 5,12-bis(phenylethynyl)naphthacene, violanthrone, 16,16-(1,2-ethylenedioxy)violanthrone, 16,17-dihexyloxyviolanthrone, 16,17-butyloxyviolanthrone, N,N′-bis(2,5-di-tert-butylphenyl)-3,4,9,10-perylenedicarboximide, 1-N,N′-dibutylaminoanthracene, 6-methylacridinium iodide, and luminol, or a derivative thereof (e.g., an acylated, alkylated, alkoxylated, and/or halogenated derivative of the compounds described herein).

In still other embodiments, the nanoparticle comprises at least about 20 mol %, 30 mol %, 40 mol %, 50 mol %, 60 mol %, 70 mol %, 80 mol %, 90 mol %, or 95 mol % of the compound of (a).

In still other embodiments, the nanoparticle comprises a matrix-forming agent selected from organic polymers, waxes, fats, oils, and surfactants, or a combination thereof.

In some embodiments, the matrix-forming agent comprises an organic polymer.

In certain embodiments, the matrix-forming agent is a non-degradable polymer (e.g., polystyrene, novolac, poly vinyl acetate, poly methyl methacrylate, poly vinyl pyrrole, poly vinyl acetate, polyisoprene, or polybutadiene).

In other embodiments, the matrix-forming agent is a polymer (e.g., a co-polymer) containing a hydrolyzable functionality (e.g., a polymer such as PLGA, PLA, or poly-ε-caprolactone). In other embodiments, the polymer is Examples of biodegradable polymers include, but are not limited to, poly(lactide), poly(glycolide), poly(orthoesters), poly(caprolactones), polylysine, poly(ethylene imine), poly(acrylic acid), poly(urethanes), poly(anhydrides), poly(esters), poly(trimethylene carbonate), poly(ethyleneimine), poly(acrylic acid), poly(urethane), poly(beta amino esters), or is a copolymer thereof (e.g., poly(lactide-co-glycolide) (PLGA)). In other embodiments, the polymer is a phospholipid.

In still other embodiments, the nanoparticle comprises a matrix-forming agent that forms an inorganic matrix. In some embodiments, the inorganic matrix-forming agent comprises iron oxide, cerium oxide, ruthenium oxide, copper oxide, copper, gold, silver, titanium dioxide, silicon, silicon nitride, tin oxide, carbon nanotubes, vanadium oxide, alumina, aluminum, cobalt oxide, platinum, palladium, zinc oxide, magnesium oxide, manganese oxide, and/or nickel oxide.

In some embodiments, the nanoparticle comprises an outer surface that comprises one or more functional groups for conjugating the nanoparticle to a binding agent.

In other embodiments, the nanoparticle further comprises an inner layer between the matrix core and the outer surface.

In certain embodiments, the binding agent comprises an antibody, ligand, protein, small molecule, an aptamer, a single-stranded nucleic acid (e.g., ssDNA or ssRNA), or a single stranded polymer nucleic acid.

In other embodiments, the nanoparticle further comprises a metalorganic compound (e.g., a metalorganic compound as described herein).

In certain embodiments, the nanoparticle has a molar ratio of the compound of (a):metalorganic compound that is about 10:1 to about 1:1, about 10:1 to about 3:1, about 8:1 to about 3:1, or about 5:1 to about 3:1.

In some embodiments, two or more surfactants are used for surfactant stabilization of the matrix.

In another aspect, the invention features a composition comprising any of the nanoparticles described herein, wherein the composition has a size distribution of nanoparticles between about 10 nm and less than about 10 μm, between about 10 nm to about 1 μm, about 10 nm to about 1 μm, about 10 nm to about 500 nm, about 10 nm to about 300 nm, or about 50 nm to about 300 nm.

In some embodiments, the composition has a size distribution of nanoparticles between about 25 nm and about 250 nm, about 25 nm and about 200 nm, about 25 nm and about 175 nm, about 25 nm and about 100 nm, or about 50 nm and about 100 nm.

In other embodiments, the composition has a polydispersity index of below about 0.35 (e.g., below about 0.25 or below about 0.15).

In another embodiment, the present invention is a nanoparticle comprising a luminophore, a luminophore precursor, chemiluminophore, a chemiluminophore precursor, a soluble absorber, or a soluble absorber precursor; one or more surfactants; and polymeric matrix-forming agents comprising a functional group, wherein the polymeric matrix-forming agents form a polymeric matrix; and wherein the compound of (a) is embedded in the matrix.

In another embodiment, the nanoparticle has a diameter between 150 nm and 200 nm.

In another embodiment, the nanoparticle has a diameter between 160 nm and 190 nm.

In another embodiment, the nanoparticle has a diameter between 170 nm and 180 nm.

In another aspect, the present invention is a method for forming the nanoparticles described herein, the method comprising
a. providing a first emulsion comprising an agent of interest, a polymeric matrix, a primary surfactant, and a first solvent system;
b. combining the first emulsion with a second solvent system to create a second emulsion;
c. mixing the second emulsion with a third solvent system to create a nanoparticle suspension; and
d. forming the water-dispersible polymeric nanoparticle in the presence of at least one secondary surfactant.

In embodiments, the invention features a liposome that includes any signal-inducing agent described herein.

In embodiments, the invention features a liposome that includes a signal-inducing agent that is any of the transition-metal catalysts described herein.

In embodiments, a liposome includes a transition metal catalyst having a structure according to formula II, formula IIA, formula IIB, formula IIIA, formula IIIB, formula IVA, formula IVB, formula V, or an oxidized or reduced form thereof.

In embodiments, a liposome includes a transition-metal catalyst having a structure selected from

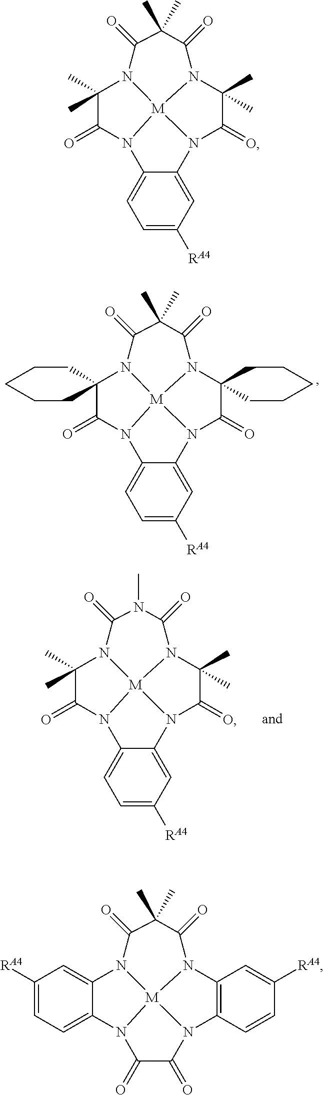

wherein R^{A4} is as described herein. In embodiments, R^{A4} is hydrogen.

In embodiments, a transition metal is selected from the group consisting of Cr, Mn, Fe, Cu, Ni and Co.

In embodiments, a transition metal is Fe (e.g., Fe(II) or Fe(III)).

In embodiments, a liposome includes an outer surface that includes one or more functional groups for conjugating a nanoparticle to a binding agent (e.g., any binding agent described herein).

In any of the methods described herein, the use of nanoparticles can be replaced with the use of liposomes (e.g., any of the liposomes described herein).

In another aspect, the present disclosure features a method for detecting an analyte, the method comprising one or more of the following steps:
(i) incubating a sample suspected of having a first analyte with a first binding agent specific to the first analyte to form a first mixture, wherein the first binding agent is conjugated to a nanoparticle or liposome comprising a first signal inducing agent, wherein the first signal inducing agent is not an enzyme if the nanoparticle or liposome contains a liquid phase; and wherein optionally the nanoparticle or liposome is free of a liquid phase;
(ii) removing from the first mixture the first binding agent that is not bound to the first analyte to form a second mixture;
(iii) dissociating the nanoparticle or liposome in the second mixture, if any, to release the first signal inducing agent into a solution, wherein the first signal inducing agent is soluble in the solution;
(iv) subjecting the first signal inducing agent to a reaction, which results in a signal change; and/or
(v) determining presence or quantity of the first analyte in the sample based on the signal change.

In some embodiments, the method further comprises, prior to step (i), incubating the sample with a second binding agent specific to the first analyte, wherein the second binding agent is immobilized on a solid support.

In another aspect, the present disclosure provides a method for detecting an analyte, comprising one or more of the following steps:
(i) incubating a solid support on which a first analyte is immobilized with a first conjugate to form a first mixture, the solid support optionally including a macroscale surface, a micro-, submicro-, or nanoparticle or a porous membrane, wherein the first conjugate comprises a first binding agent specific to the first analyte and a first nanoparticle or liposome that comprises a first signal inducing agent, wherein the first signal inducing agent is not an enzyme if the first nanoparticle contains a liquid phase, and optionally wherein the first nanoparticle or liposome is free of a liquid phase;
(ii) removing from the first mixture unbound first conjugate to form a second mixture;
(iii) dissociating the first nanoparticle or liposome in the first conjugate to release the first signal inducing agent into a solution, wherein the first signal inducing agent is soluble in the solution;
(iv) subjecting the first signal inducing agent to a reaction, which results in a signal change; and/or
(v) determining presence or quantity of the first analyte in the sample based on the signal change.

In some embodiments, the method may further comprise, prior to step (i), incubating the solid support with a sample suspected of containing the first analyte to allow for immobilization of the first analyte onto the solid support.

In some examples, step (i) is performed in the presence of the first binding agent in free form.

Alternatively, the method may further comprise, prior to step (i), incubating a sample suspected of having the first analyte with the first conjugate.

In any of the methods described herein, the first binding agent and/or the second binding agent can be an antibody; a nucleic acid (e.g., a single-stranded DNA or RNA or an aptamer, or a polymer nucleic acid), and a member of a receptor-ligand pair. In some examples, the first binding agent and the second binding agent are antibodies binding to the analyte, and wherein the first and second binding agents bind to different epitopes of the analyte.

Any of the nanoparticles described herein may comprise an outer surface that comprises one or more functional groups for conjugating the nanoparticle to the first binding agent. Optionally, it may further comprise an impermeable layer underneath the outer surface, wherein the impermeable layer blocks diffusion of the first signal inducing agent from the nanoparticle.

In another aspect, the present disclosure provides a method for detecting an analyte, comprising one or more of the following steps:
(i) incubating a sample suspected of having an analyte of interest with a binding agent specific to the analyte under conditions that permit binding between the analyte and the binding agent; wherein the binding agent is associated with a nanoparticle or liposome comprising a signaling agent; wherein the signaling agent is not an enzyme; and wherein if the signaling agent is a pre-chemiluminophore, the nanoparticle is not crystalline.
(ii) dissociating the nanoparticle or liposome bound to the analyte, if any, to release the signaling agent such that it results in a signal change; and
(iii) determining presence or quantity of the analyte in the sample based on the signal change.

In another aspect, the present disclosure provides a method for detecting an analyte, comprising one or more of the following steps:
(i) incubating a sample suspected of having an analyte of interest with a binding agent specific to the analyte under conditions that permit binding between the analyte and the binding agent; wherein the binding agent is associated with a nanoparticle or liposome comprising a signaling agent; wherein the signaling agent is not an enzyme; and further wherein the binding agent is associated with the nanoparticle or liposome via an interaction other than an electrostatic interaction.
(ii) dissociating the nanoparticle or liposome bound to the analyte, if any, to release the signaling agent such that it results in a signal change; and
(iii) determining presence or quantity of the analyte in the sample based on the signal change.

In embodiments, a binding agent is selected from antibodies or antigen-binding fragments thereof, enzymes, oligonucleotides, DNA, RNA, PNA, or LNA, proteins, peptides, polypeptides, receptors, ligands, small molecules, aptamers, polysaccharides, plastibodies, affibodies, camelids, fibronectins, or a combination thereof.

In embodiments, a binding agent is an antibody or antigen-binding fragment thereof.

In embodiments, an antibody or antigen-binding fragment thereof is a primary antibody or a secondary antibody.

In embodiments, a binding agent is a small molecule.

In embodiments, a binding agent is associated with the nanoparticle or liposome via covalent conjugation, non-covalent interaction, and/or adsorption.

In embodiments, a binding agent is associated with the nanoparticle or liposome via covalent conjugation.

In embodiments, the dissociating step comprises treating the nanoparticle or liposome with a physical trigger, a chemical trigger, or a combination thereof.

In embodiments, the physical trigger is selected from the group consisting of thermal energy, electromagnetic energy, and/or sound energy.

In embodiments, the chemical trigger is an enzyme, a catalyst, a solvent, or an acid or base or other chemical agent, or a combination thereof.

In embodiments, step (ii) and step (iii) are performed simultaneously in a solution.

In embodiments, the solution further comprises a chemical trigger for dissociating the nanoparticle.

In embodiments, the solution further comprises a pH modulator, a solvent, a catalyst, a co-catalyst, or a combination thereof.

In embodiments, the sample is a biological sample.

In embodiments, the biological sample is selected from cells, cell lysate, FFPE (FASP Protein Digestion) digests, tissues including tissue biopsies or autopsy samples, whole blood, plasma, serum, urine, stool, saliva, cerebrospinal fluid, cord blood, chorionic villus samples amniotic fluid, and transcervical lavage fluid.

In another aspect, the invention features a kit for detecting an analyte, comprising
(i) a binding agent specific to the analyte, wherein the binding agent is associated with a nanoparticle or liposome comprising a signaling agent; wherein the signaling agent is not an enzyme; and wherein if the signaling agent is a pre-chemiluminophore, the nanoparticle is not crystalline; and
(ii) a solution comprising reagents for performing a reaction that results in a signal change, once the signaling agent is released from the nanoparticle or liposome.

In another aspect, the invention features a kit for detecting an analyte, comprising
(i) a binding agent specific to the analyte, wherein the binding agent is associated with a nanoparticle or liposome comprising a signaling agent; wherein the signaling agent is not an enzyme; and further wherein the binding agent is associated with the nanoparticle via an interaction other than an electrostatic interaction; and
(ii) a solution comprising reagents for performing a reaction that results in a signal change, once the signaling agent is released from the nanoparticle or liposome.

In another aspect, the invention features a kit for detecting an analyte, comprising
(i) a nanoparticle or liposome comprising a signaling agent and one or more functional groups for associating the nanoparticle or liposome to a binding agent specific for an analyte; and
(ii) a solution comprising reagents for performing a reaction that results in a signal change, once the signaling agent is released from the nanoparticle or liposome.

In embodiments, the signaling agent is not an enzyme.

In embodiments, when the signaling agent is a pre-chemiluminophore, the nanoparticle is not crystalline.

In embodiments, one or more functional groups are designed for covalent conjugation, non-covalent interaction, and/or adsorption.

### BRIEF DESCRIPTION OF DRAWINGS

FIGS. 1A-D include diagrams illustrating exemplary designs of nanoparticles comprising signal inducing agents (payloads). FIG. 1A illustrates payloads embedded in polymer matrixes. FIG. 1B illustrates nanoparticles in core-shell format comprising heterogeneous distributed payloads. FIG. 1C illustrates nanoparticles in core-shell format comprising homogeneously distributed payloads. FIG. 1D illustrates nanoparticles comprising antibodies on the surface as binding agents and having payloads entrapped.

FIGS. 2A-B include diagrams showing amplification assay format. FIG. 2A shows one tier amplification. FIG. 2B shows two-tier amplification.

FIG. 3 shows that the fluorescent signals of both fluorescein dilaurate dependent fluorescein and TAML-dependent resorufin are correlated with the concentration of S1131 nanoparticle.

FIG. 4 is a plot illustrating the signal strength of newly synthesized nanoparticles compared to nanoparticles stored at room temperature for four months for use in a cTnl ELISA assay.

FIG. 5 is a plot illustrating the reflective fluorescence unit intensity (RFU) as compared to the number of DNA copies made in a DNA-hybridization assay.

FIG. 6 is a plot illustrating the normalized optical signal from a sandwich immunoassay for human C-reactive protein (CRP).

FIG. 7 provides data regarding non-specific binding by nanoparticles to three surfaces including: non-treated base plastic surface, plastic surface pretreated with PBS, and plastic surface pretreated with PBS containing 1% bovine serum albumin (BSA).

FIG. 8 provides data regarding non-specific binding by nanoparticles to two surfaces.

FIG. 9 relates to use of nanoparticles in a human adiponectin ELISA study.

FIG. 10 is a schematic illustrating the synthesis of an example nanoparticle.

FIG. 11 illustrates the relationship between particle size and concentration of FDL present in the nanoparticle.

FIG. 12 illustrates the effect of nanoparticle size for nanoparticles comprising fluorescein dilaurate binding.

FIG. 13 illustrates the effect nanoparticle functional group concentration on specific and nonspecific binding.

FIG. 14 is a plot illustrating the logarithmic relationship between the concentration of target sample (P4) and the presence of bound nanoparticles as measured in relative fluorescence units (RFU).

FIG. 15 is a plot illustrating the strength of the fluorescent signal, measured in relative fluorescent units (RFU), of a nanoparticle comprising fluorescein dilaurate compared to the concentration of human chorionic gonadotropin (hCG) in an assay. The nanoparticles used in this example had been aged for at least four months.

FIG. 16 shows a schematic of the nanoparticle fabrication methods.

FIG. 17 shows the tuning of the shell region to enable effective encapsulation of a water-soluble salt, and the metal-tetraamidomacrocyclic ligand complex.

FIGS. 18A-18D show representative data obtained from Nanosight measurements for the formulations prepared according to Example 6.5 (FIG. 18A); Example 6.6 (FIG. 18B); Example 6.7 (FIG. 18C); and Example 6.8 (FIG. 18D).

### DETAILED DESCRIPTION OF THE INVENTION

Described herein are nanoparticles comprising a transition-metal catalyst and one or more matrix-forming agents providing a dissociable matrix. In some embodiments, the matrix sequesters the transition-metal catalyst until said matrix is dissociated. In further embodiments, the transition-metal catalyst is embedded in the matrix without being primarily governed by electrostatic interactions (e.g., the catalyst is embedded in the matrix via van der Waals interactions or by stabilization by one or more surfactants during formation of the matrix).

Upon dissociation of the matrix in solution, the transition-metal catalyst can then effect a reaction with a substrate in solution (e.g., an oxidation reaction or cleavage of a fluorescence quenching group) that induces a measurable effect (e.g., an optically-detectable signal such as a decrease or an increase in fluorescence). Improved properties, e.g., improved signal amplification, can be obtained by the use of transition-metal catalysts having high turnover rate and/or adjusting the loading of the transition-metal catalyst in the nanoparticle.

Also described herein are nanoparticles comprising a compound that is chemiluminophore, a chemiluminophore precursor, a soluble absorber, or a soluble absorber precursor and one or more matrix-forming agents providing a matrix. In some embodiments, the loading of the chemiluminophore or the soluble absorber, or the derivative thereof, can allow for sensitivity in the detection of small amounts of analytes.

Upon dissociation of the matrix in solution, a chemiluminophore or a soluble absorber, or a derivative thereof, can either directly provide a measurable signal or undergo a reaction (e.g., a bond cleavage such as an ester cleavage) that provides a measurable signal (e.g., an optically-detectable signal such as a decrease or an increase in fluorescence).

The invention also features assay methods for detecting or quantifying one or more analytes in a sample, wherein the assay methods involve the use of nanoparticles or liposomes that comprise one or more signal inducing agents. As used herein, a “signal inducing agent” is an agent that is capable of reacting physically or chemically with itself or another substrate to produce a detectable signal. The detectable signal can be, for example, a fluorescent signal or an electrical signal. A nanoparticle can comprise one or more binding agents specific to an analyte of interest. When binding to the analyte, the nanoparticle can be dissociated (e.g., by a physical or chemical trigger) to release the transition-metal catalyst into solution in which the substrate is soluble. The solution can be a pure solvent, or a mixture of one or more solvent and one or more solutes. The substrate is then subjected to a reaction, leading to a signal change (e.g., increase a signal or reduce a signal). The presence or quantify of the analyte of interest can be determined based on the signal change.

### I. DEFINITIONS

Compounds suitable for use in this invention include, but are not limited to, those described herein for the transition-metal catalysts and substrates, and are further illustrated by the classes, subclasses, and species disclosed herein. Other compounds suitable for use in this invention include, but are not limited to, those described herein for a chemiluminophore or a soluble absorber, or a derivative thereof, as well as exemplary transition-metal catalysts and substrates, and are further illustrated by the classes, subclasses, and species disclosed herein.

It will be appreciated that preferred subsets described for each variable herein can be used for any of the structural subsets as well. As used herein, the following definitions shall apply unless otherwise indicated.

As described herein, the compounds described herein may be optionally substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which hydrogen atom can be replaced with the radical of a suitable substituent. Unless otherwise indicated, an “optionally substituted” group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

A stable compound or chemically feasible compound is one in which the chemical structure is not substantially altered when kept at a temperature from about −80° C. to about +40°, in the absence of moisture or other chemically reactive conditions, for at least a week, or a compound which maintains its integrity long enough to be useful for therapeutic or prophylactic administration to a patient.

The phrase “one or more substituents”, as used herein, refers to a number of substituents that equals from one to the maximum number of substituents possible based on the number of available bonding sites, provided that the above conditions of stability and chemical feasibility are met.

As used herein, the term “about” in relation to a numerical value x means, for example, x+10%.

As used herein, the term “independently selected” means that the same or different values may be selected for multiple instances of a given variable in a single compound.

As used herein, the term “aromatic” includes aryl and heteroaryl groups as described generally below and herein.

The term “aliphatic” or “aliphatic group”, as used herein, means an optionally substituted straight-chain or branched C₁₋₁₂ hydrocarbon which is completely saturated or which contains one or more units of unsaturation. For example, suitable aliphatic groups include optionally substituted linear or branched alkyl, alkenyl, and alkynyl groups. Unless otherwise specified, in various embodiments, aliphatic groups have 1-12, 1-10, 1-8, 1-6, 1-4, 1-3, or 1-2 carbon atoms. It is apparent to a skilled person in the art that in some embodiments, the “aliphatic” group described herein can be bivalent.

The term “alkyl”, used alone or as part of a larger moiety, refers to a saturated, optionally substituted straight or branched chain hydrocarbon group having 1-12, 1-10, 1-8, 1-6, 1-4, 1-3, or 1-2 carbon atoms.

The term “alkenyl”, used alone or as part of a larger moiety, refers to an optionally substituted straight or branched chain hydrocarbon group having at least one double bond and having 2-12, 2-10, 2-8, 2-6, 2-4, or 2-3 carbon atoms.

The term “alkynyl”, used alone or as part of a larger moiety, refers to an optionally substituted straight or branched chain hydrocarbon group having at least one triple bond and having 2-12, 2-10, 2-8, 2-6, 2-4, or 2-3 carbon atoms.

The terms “cycloaliphatic”, “carbocycle”, “carbocyclyl”, “carbocyclo”, or “carbocyclic”, used alone or as part of a larger moiety, refer to an optionally substituted saturated or partially unsaturated cyclic aliphatic ring system having from 3 to about 14 ring carbon atoms. In some embodiments, the cycloaliphatic group is an optionally substituted monocyclic hydrocarbon having 3-6, 3-8, or 3-10 ring carbon atoms. Cycloaliphatic groups include, without limitation, optionally substituted cyclopropyl, cyclobutyl, cyclopentyl, cyclopentenyl, cyclohexyl, cyclohexenyl, cycloheptyl, cycloheptenyl, cyclooctyl, cyclooctenyl, or cyclooctadienyl. The terms “cycloaliphatic”, “carbocycle”, “carbocyclyl”, “carbocyclo”, or “carbocyclic” also include optionally substituted bridged or fused bicyclic rings having 6-12, 6-10, or 6-8 ring carbon atoms, wherein any individual ring in the bicyclic system has 3-8 ring carbon atoms.

The term “cycloalkyl” refers to an optionally substituted saturated ring system of about 3 to about 10 ring carbon atoms. Exemplary monocyclic cycloalkyl rings include cyclopropyl, cyclobutyl, cyclopentyl, cyclohexyl, and cycloheptyl.

The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentyl, cyclohexenyl, cycloheptenyl, and cyclooctenyl.

The term “halogen” or “halo” means F, Cl, Br, or I.

The term “heteroatom” refers to one or more of oxygen, sulfur, nitrogen, phosphorus, and silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The terms “aryl” and “ar-”, used alone or as part of a larger moiety, e.g., “aralkyl”, “aralkoxy”, or “aryloxyalkyl”, refer to an optionally substituted C₆₋₁₄aromatic hydrocarbon moiety comprising one to three aromatic rings. For example, the aryl group is a C₆₋₁₀ aryl group (i.e., phenyl and naphthyl). Aryl groups include, without limitation, optionally substituted phenyl, naphthyl, or anthracenyl. The terms “aryl” and “ar-”, as used herein, also include groups in which an aryl ring is fused to one or more cycloaliphatic rings to form an optionally substituted cyclic structure such as a tetrahydronaphthyl, indenyl, or indanyl ring. The term “aryl” may be used interchangeably with the terms “aryl group”, “aryl ring”, and “aromatic ring”.

An “aralkyl” or “arylalkyl” group comprises an aryl group covalently attached to an alkyl group, either of which independently is optionally substituted. For example, the aralkyl group is C₆₋₁₀ arylC₁₋₆alkyl, including, without limitation, benzyl, phenethyl, and naphthylmethyl.

The terms “heteroaryl” and “heteroar-”, used alone or as part of a larger moiety, e.g., “heteroaralkyl”, or “heteroaralkoxy”, refer to groups having 5 to 14 ring atoms, preferably 5, 6, 9, or 10 ring atoms; having 6, 10, or 14 π electrons shared in a cyclic array; and having, in addition to carbon atoms, from one to five heteroatoms. A heteroaryl group may be mono-, bi-, tri-, or polycyclic, for example, mono-, bi-, or tricyclic (e.g., mono- or bicyclic). The term “heteroatom” refers to nitrogen, oxygen, or sulfur, and includes any oxidized form of nitrogen or sulfur, and any quaternized form of a basic nitrogen. For example, a nitrogen atom of a heteroaryl may be a basic nitrogen atom and may also be optionally oxidized to the corresponding N-oxide. When a heteroaryl is substituted by a hydroxy group, it also includes its corresponding tautomer. The terms “heteroaryl” and “heteroar-”, as used herein, also include groups in which a heteroaromatic ring is fused to one or more aryl, cycloaliphatic, or heterocycloaliphatic rings. Nonlimiting examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 3- to 8-membered monocyclic or 7-10-membered bicyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. Additionally, a heterocyclic ring also includes groups in which the heterocyclic ring is fused to one or more aryl rings.

As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

As used herein, the term “bivalent C_{x-y} (e.g., C₁-6) saturated or unsaturated, straight or branched, hydrocarbon chain”, refers to bivalent alkylene, alkenylene, and alkynylene chains that are straight or branched as defined herein.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocycylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkenylene”, a bivalent alkynyl chain is “alkynylene”, and so forth.

The term “alkylene” refers to a bivalent alkyl group. An “alkylene chain” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3-7-membered ring. The substituents can be on the same or different atoms.

An alkylene chain also can be optionally interrupted by a functional group. An alkylene chain is “interrupted” by a functional group when an internal methylene unit is interrupted or replaced by the functional group. Examples of suitable “interrupting functional groups” are described in the specification and claims herein.

For purposes of clarity, and unless otherwise stated, all bivalent groups described herein, including, e.g., the alkylene chain linkers described above, are intended to be read from left to right, with a corresponding left-to-right reading of the formula or structure in which the variable appears.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group. A substituted alkynylene chain is a polymethylene group containing at least one triple bond in which one or more hydrogen atoms are replaced with a substituent. Suitable substituents include those described below for a substituted aliphatic group.

As used herein, the term “cycloalkylenyl” refers to a bivalent cycloalkyl group of the following structure:

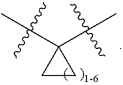

An aryl (including aralkyl, aralkoxy, aryloxyalkyl and the like) or heteroaryl (including heteroaralkyl and heteroarylalkoxy and the like) group may contain one or more substituents and thus may be “optionally substituted”. In addition to the substituents defined above and herein, suitable substituents on the unsaturated carbon atom of an aryl group (e.g., phenyl or naphthyl) or heteroaryl group (e.g., pyridyl) also include and are generally selected from -halo, —NO₂, —CN, —R⁺, —C(R+)═C(R+)₂, —C≡C—R⁺, —OR⁺, —SR^{o}, —S(O)R^{o}, —SO₂R^{o}, —SO₃R⁺, —SO₂N(R⁺)₂, —N(R⁺)₂, —NR⁺C(O)R⁺, —NR⁺C(S)R⁺, —NR⁺C(O)N(R⁺)₂, —NR⁺C(S)N(R⁺)₂, —N(R⁺)C(═NR⁺)—N(R⁺)₂, —N(R⁺)C(═NR⁺)—R^{o}, —NR⁺CO₂R⁺, —NR⁺SO₂R^{o}, —NR⁺SO₂N(R⁺)₂, —O—C(O)R⁺, —O—CO₂R⁺, —OC(O)N(R⁺)₂, —C(O)R⁺, —C(S)R^{o}, —CO₂R⁺, —C(O)—C(O)R⁺, —C(O)N(R⁺)₂, —C(S)N(R⁺)₂, —C(O)N(R⁺)—OR⁺, —C(O)N(R⁺)C(═NR⁺)—N(R⁺)₂, —N(R⁺)C(═NR⁺)—N(R⁺)—C(O)R⁺, —C(═NR⁺)—N(R⁺)₂, —C(═NR⁺)—OR⁺, —N(R⁺)—N(R⁺)₂, —C(═NR⁺)—N(R⁺)—OR⁺, —C(R^{o})═N—OR⁺, —P(O)(R⁺)₂, —P(O)(OR⁺)₂, —O—P(O)—OR⁺, and —P(O)(NR⁺)—N(R⁺)₂, wherein R⁺, independently, is hydrogen or an optionally substituted aliphatic, aryl, heteroaryl, cycloaliphatic, or heterocyclyl group, or two independent occurrences of R⁺ are taken together with their intervening atom(s) to form an optionally substituted 5-7-membered aryl, heteroaryl, cycloaliphatic, or heterocyclyl ring. Each R^{o} is an optionally substituted aliphatic, aryl, heteroaryl, cycloaliphatic, or heterocyclyl group.

An aliphatic or heteroaliphatic group, or a non-aromatic carbocyclic or heterocyclic ring may contain one or more substituents and thus may be “optionally substituted”. Unless otherwise defined above and herein, suitable substituents on the saturated carbon of an aliphatic or heteroaliphatic group, or of a non-aromatic carbocyclic or heterocyclic ring are selected from those listed above for the unsaturated carbon of an aryl or heteroaryl group and additionally include the following: ═O, ═S, ═C(R*)₂, ═N—N(R*)₂, ═N—OR*, ═N—NHC(O)R*, ═N—NHCO₂R^{o}═N—NHSO₂R^{o} or ═N—R* where R^{o} is defined above, and each R* is independently selected from hydrogen or an optionally substituted C₁₋₆ aliphatic group.

In addition to the substituents defined above and herein, optional substituents on the nitrogen of a non-aromatic heterocyclic ring also include and are generally selected from

—R⁺, —N(R⁺)₂, —C(O)R⁺, —C(O)OR⁺, —C(O)C(O)R⁺, —C(O)CH₂C(O)R⁺, —S(O)₂R⁺, —S(O)₂N(R⁺)₂, —C(S)N(R⁺)₂, —C(═NH)—N(R⁺)₂, or —N(R⁺)S(O)₂R⁺; wherein each R⁺ is defined above. A ring nitrogen atom of a heteroaryl or non-aromatic heterocyclic ring also may be oxidized to form the corresponding N-hydroxy or N-oxide compound. A nonlimiting example of such a heteroaryl having an oxidized ring nitrogen atom is N-oxidopyridyl.

As detailed above, in some embodiments, two independent occurrences of R⁺ (or any other variable similarly defined in the specification and claims herein), are taken together with their intervening atom(s) to form a monocyclic or bicyclic ring selected from 3-13-membered cycloaliphatic, 3-12-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur.

Exemplary rings that are formed when two independent occurrences of R⁺ (or any other variable similarly defined in the specification and claims herein), are taken together with their intervening atom(s) include, but are not limited to the following: a) two independent occurrences of R⁺ (or any other variable similarly defined in the specification or claims herein) that are bound to the same atom and are taken together with that atom to form a ring, for example, N(R⁺)₂, where both occurrences of R⁺ are taken together with the nitrogen atom to form a piperidin-1-yl, piperazin-1-yl, or morpholin-4-yl group; and b) two independent occurrences of R⁺ (or any other variable similarly defined in the specification or claims herein) that are bound to different atoms and are taken together with both of those atoms to form a ring, for example where a phenyl group is substituted with two occurrences of OR⁺

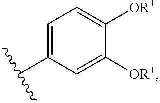

these two occurrences of R⁺ are taken together with the oxygen atoms to which they are bound to form a fused 6-membered oxygen containing ring:

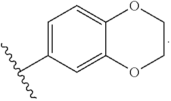 [SMILES: *c1ccc2c(c1)OCCO2]

It will be appreciated that a variety of other rings (e.g., spiro and bridged rings) can be formed when two independent occurrences of R⁺ (or any other variable similarly defined in the specification and claims herein) are taken together with their intervening atom(s) and that the examples detailed above are not intended to be limiting.

As used herein, R^{Z} is hydrogen, halogen, or —X^{Z1}—Y^{Z1}, —Z^{Z1}, wherein X^{Z1} is —C(R^{XZ1})₂—, —C(O)—, —C(O)O—, —C(O)NH—, —CR^{XZ1}═CR^{XZ1}—, —NR^{XZ1}—, —NR^{XZ1}C(O)—, —O—, or —OC(O)—, wherein R^{XZ1} is hydrogen or C₁₋₆ alkyl; Y^{Z1} is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y^{Z1} are optionally and independently replaced by -Cy^{Z1}-, NR^{YZ1-}, —N(R^{YZ1})C(O)—, —C(O)N(R^{YZ1})—, —O—, —C(O)—, —OC(O)—, —C(O)O—, or —N═N—, wherein R^{YZ1} is hydrogen or C₁₋₆ alkyl; and each Cy^{Z1} is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and Z^{Z1} is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl. In an example embodiment, R^{Z} is —O—CH₂—CH═CH₂.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures where there is a replacement of hydrogen by deuterium or tritium, or a replacement of a carbon by a ¹³C- or ¹⁴C-enriched carbon are within the scope of this invention. Such compounds are useful, as a nonlimiting example, as analytical tools or probes in biological assays.

It is to be understood that, when a disclosed compound has at least one chiral center, the present invention encompasses one enantiomer of inhibitor free from the corresponding optical isomer, racemic mixture of the inhibitor and mixtures enriched in one enantiomer relative to its corresponding optical isomer. When a mixture is enriched in one enantiomer relative to its optical isomers, the mixture contains, for example, an enantiomeric excess of at least 50%, 75%, 90%, 95% 99% or 99.5%.

The enantiomers of the present invention may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. Where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a disclosed compound has at least two chiral centers, the present invention encompasses a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s). When a mixture is enriched in one diastereomer or diastereomeric pair(s) relative to the other diastereomers or diastereomeric pair(s), the mixture is enriched with the depicted or referenced diastereomer or diastereomeric pair(s) relative to other diastereomers or diastereomeric pair(s) for the compound, for example, by a molar excess of at least 50%, 75%, 90%, 95%, 99% or 99.5%.

The diastereoisomeric pairs may be separated by methods known to those skilled in the art, for example chromatography or crystallization and the individual enantiomers within each pair may be separated as described above. Specific procedures for chromatographically separating diastereomeric pairs of precursors used in the preparation of compounds disclosed herein are provided the examples herein.

The symbol [symbol] , as used herein, represents a point of attachment between two atoms.

### II. NANOPARTICLES AND LIPOSOMES

The nanoparticles or liposomes for use in any of the assay methods described herein can be made of a suitable material such that the nanoparticles or liposomes can be dissociated under, e.g., a chemical trigger. The suitable trigger for dissociating a particular nanoparticle or liposome would depend on the materials used for making the nanoparticle, which is within the knowledge of a skilled person in the art.

In some embodiments, the nanoparticle described herein is in a single phase format which comprises a core structure (e.g., a matrix) and a functional surface. The core structure (e.g., a matrix) can be made of any suitable material(s) as known in the art or disclosed herein. A signal inducing agent as described herein is embedded or encapsulated in the core structure (e.g., a matrix). The functional surface is for conjugating to a binding agent specific to an analyte of interest.

The core structure (e.g., a matrix) may comprise polymers, waxes, surfactants, and/or lipids. In some embodiments, the core structure (e.g., a matrix) can be made of natural and/or synthetic waxes, e.g., carnauba, beeswax, paraffin, microcrystalline, candle, siliconyl, Kester wax, candelilla, jojoba wax, or rice bran wax. Alternatively or in addition, the core structure (e.g., a matrix) may comprise fatty alcohols and fatty acids: cetyl alcohol, palmitoleyl alcohol, stearyl alcohol, nonadecyl alcohol, heptadecyl alcohol, propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, linolenic acid, stearidonic acid, linoleic acid, palmitoleic acid, oleic acid, or a combination thereof.

In other embodiments, the core structure (e.g., a matrix) may comprise nondegradable polymers (e.g., polystyrene, novolac, poly vinyl acetate, poly methyl methacrylate, poly vinyl pyrrole, poly vinyl acetate, polyisoprene, polybutadiene) and/or degradable polymers (e.g., PLGA, PLA, poly-ε-caprolactone, or polyethylene glycol). The polymer can include a region that is positively charged, such as, for example, Poly(vinyl alcohol), N-methyl-4(4′-formylstyryl)pyridinium methosulfate acetal.

In an example embodiment, polymers, lipids, and surfactants useful in the present invention comprise a hydrophobic end and a hydrophilic end. Accordingly, in some embodiments, a matrix-forming agent can be a polymer comprising a functional group. In another embodiment, a polymer comprises a hydrophobic region and a hydrophilic region. In another embodiment, a functional group is located in the hydrophilic region. Suitable examples include polymers represented by the following structural formula:

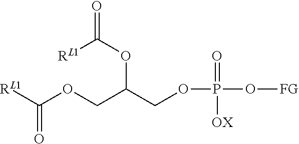

wherein R^{L1} is a C₁₋₆₀ alkyl, C₂₋₆₀ alkenyl, C₂₋₆₀ alkynyl, FG represents a functional group, and X represents a suitable counter ion (e.g., sodium, potassium, or ammonium). In another embodiment, a polymer is a fatty acid based polymer. In one example embodiment, a fatty acid based polymer is a phospholipid. In another example embodiment, the phospholipid is represented by the following structural formula:

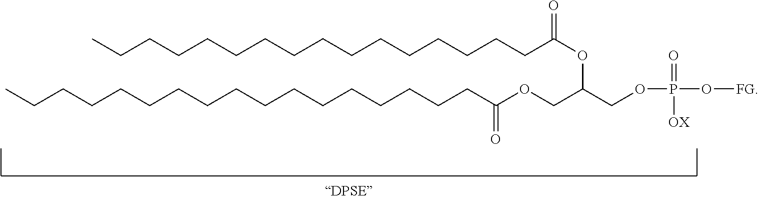

In one embodiment, FG is selected from: maleimidyl, thiolyl, hydrazidyl, tetrazinyl, trans-cyclooctenyl,

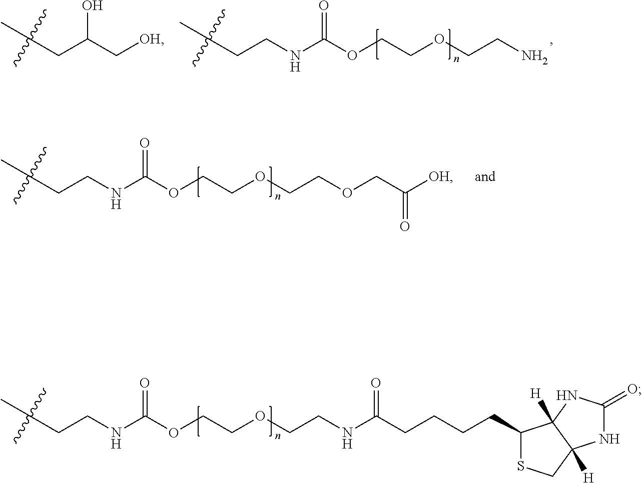

wherein n is an integer greater than 1; and [symbol] represents a point of attachment between two atoms. Any combination of the polymers and functional groups described herein can be used as matrix-forming agents.

In an alternative embodiment, the fatty acid based polymer is selected from 1,2-distearoyl-sn-glycero-3-phosphoethanolamine-N-[amino(polyethylene glycol)-2000], 1,2-distearoyl-sn-glycero-3-phosphoethanolamine-N-[methoxy(polyethylene glycol)-2000], 1,2-distearoyl-sn-glycero-3-phosphoethanolamine-N-[azido(polyethylene glycol)-2000], 1,2-distearoyl-sn-glycero-3-phosphoethanolamine-N-[carboxy(polyethylene glycol)-2000], 1,2-distearoyl-sn-glycero-3-phosphoethanolamine-N-[biotinyl(polyethylene glycol)-2000], 1,2-distearoyl-sn-glycero-3-phosphoethanolamine-N-[maleimide(polyethylene glycol)-2000], and 1,2-distearoyl-sn-glycero-3-phosphoethanolamine-N-[succinyl(polyethylene glycol)-2000], and salts thereof. Any combination of the fatty acid based polymers can be used in embodiments of the present invention.

In another embodiment, alternatively or in addition to the matrix-forming agents described above, the matrix-forming agent is a surfactant. In one embodiment the surfactant is a small surfactant. In another embodiment, a small surfactant can be anionic, cationic, nonionic, or zwitterionic. Examples of suitable anionic surfactants include, but are not limited to, carboxylates (e.g., palmitic acid, valeric acid, lauric acid, sodium stearate, and sodium cholate hydrate); sulfonates (e.g., perfluorooctanesulfonate); phosphates (e.g., polyoxyethylene tristyrylphenol phosphate); and sulfates (e.g., sodium dodecyl sulfate, sodium laureth sulfate, sodium lauryl ether sulfate, and sodium palmityl sulfate). Examples of suitable cationic surfactants include, but are not limited to benzyldimethylhexadecylammonium chloride, hexadecyltrimethylammonium bromide, 1-bromotetradecane, myristyltrimethylammonium bromide, and methyltrialkyl(C₈₋₁₀) ammonium chloride (Adogen® 464). Examples of suitable nonionic surfactants include, but are not limited to oleyl alcohol, Triton X-100, cocamide MEA, and dodecyldimethylamine oxide. Examples of suitable zwitterionic surfactants include, but are not limited to N-dodecyl-N,N-dimethyl-3-ammonio-1-propanesulfonate, 1,2-dimyristoyl-sn-glycero-3-phosphocholine, and 1,2-dimyristoyl-sn-glycero-3-phosphoethanolamine.

In another embodiment, the surfactant is a polymer-based surfactant. In another embodiment, the polymer-based surfactant is a poly(lactic acid) based polymer.

In another embodiment, the polymer-based surfactant is a poly(lactic acid) based polymer. Suitable examples of poly(lactic acid) based polymers include, but are not limited to methoxy (polyethylene glycol)-b-poly(L-lactide), methoxy poly(ethylene glycol)-b-poly(D,L-lactide), methoxy poly(ethylene glycol)-b-poly(lactide-co-glycolide), poly(D,L-lactide)-b-poly(ethylene glycol)-carboxylic acid, poly(D,L-lactide)-b-poly(ethylene glycol)-maleimide, poly(D,L-lactide-co-glycolide)-b-poly(ethylene glycol)-carboxylic acid, poly(D,L-lactide)-NH₂ (diamine), azide-poly(ethylene glycol)-amine, azide-poly(ethylene glycol)-thiol, azide-poly(ethylene glycole)-carboxylic acid, and thiol poly(ethylene glycol) amine. Any combination of the poly(lactic acid) based polymers can be used in embodiments of the present invention.

In some embodiments, the core structure (e.g., a matrix) may comprise one or more inorganic compounds, which may form a matrix. The signal inducing agent can be embedded in the matrix. Example inorganic compounds for use in the present disclosure include, but are not limited to, iron oxide, cerium oxide, ruthenium oxide, copper oxide, copper, gold, silver, titanium dioxide, silicon, silicon nitride, tin oxide, carbon nanotubes, vanadium oxide, alumina, aluminum, cobalt oxide, platinum, palladium, zinc oxide, magnesium oxide, manganese oxide, nickel oxide.

In yet other embodiments, the core structure (e.g., a matrix) may be made of a material that can also serve as a signal inducing agent as described herein. Examples include a metal ion, a metal oxide, a metalorganic compound, a fluorophore, a chemiluminophore, and/or a photosensitizer.

In some embodiments, the core structure (e.g., a matrix) may comprise a dopant. A dopant is a trace element inserted into a substance in order to alter the chemical, thermal, optical, magnetic, and/or electrical properties of the substance. In the presence disclosure, a dopant is used to enhance the disassociation of the nanoparticles to release the signal inducing agent contained therein under a trigger, such as a physical trigger. The dopant may be a light-sensitive molecule, which was known in the art. Examples include diazonaphthoquinone (DNQ) and its derivatives, for example, esters of DNQ (as known in the area of photoresists). The dopant may also be a thermally-absorbing species, such as metallic nanoparticles, e.g. gold, silver, aluminum, nickel.

Any of the core structures (e.g., a matrix) described herein may also comprise one or more surfactants, including, but not limited to, Brijs, Spans, Tweens, Tritons, Igepals, Pluoronics, Poloxamers, lecithin, glyceryl monostearate, glyceryl monooleate, glyceryl monothioglycolate, glyceryl monocaprylate, glyceryl monolaurate, 2-cyano-2-propyl dodecyl trithiocarbonate, 1,4-phenylene dimethacrylate, compritol 888 or a combination thereof.

The nanoparticle as described herein contains a functional outer surface which may coat the core structure (e.g., a matrix) directly or indirectly. The outer surface may comprise modified surfactant with functional surface and/or a mix of surfactant and surfactant with modified surface. Examples of the surfactants include, but are not limited to, Brijs, Spans, Tweens, Tritons, Igepals, Pluoronics, Poloxamers, lecithin, glyceryl monostearate, glyceryl monooleate, glyceryl monothioglycolate, glyceryl monocaprylate, glyceryl monolaurate; functional surfaces may include amine, carboxylic acids, thiol, azides, alkynes, Ni, histidines, Cu, lysines, maleimide, NHS-ester, biotin, avidin, or a combination thereof.

In some instances, an intermediate agent is conjugated to the nanoparticle via the functional surface. The intermediate agent can bind to the binding agent either directly or indirectly. In one example, a biotin is conjugated to the functional surface of the nanoparticle as an intermediate agent. A biotin-conjugated binding agent can then be attached to the nanoparticle via a streptavidin.

In some embodiments, the nanoparticle may further comprise one or more stabilizing layers between the core structure (e.g., a matrix) and the functional outer surface. The stabilizing layer may comprise poly ethylene glycol (PEG) or a similar hydrophilic polymer-modified surface. Nanoparticle anchoring may occur with a hydrophobic region of the polymer forming a block-copolymer, which may be further designed to include a functional group at the end cap of the hydrophilic polymer. The layer may comprise an impermeable layer, alone or in combination with other layers of the nanoparticle, that may inhibit the release of the signal inducing agent from the nanoparticle to the environment before dissociation of the nanoparticle. The stabilizing layers may be applied deterministically or may self-assemble.

The nanoparticle can be in a matrix format, in which the transition-metal catalyst is embedded or entrapped. Alternatively, the nanoparticle may be in a core-shell format, in which the signal inducing agent is encapsulated.

For example, any of the core structures (e.g., a matrix) described herein containing one or more signal inducing agents may be coated with a layer (a capping layer), which can be made of the same polymer material(s) as the core structure (e.g., a matrix). The outer functional surface as described herein is added on top of the capping layer. Such a nanoparticle may further comprise one or more stabilizing layer as described herein between the capping layer and the outer surface.

In some embodiments, the nanoparticle may be in a liposome format, which comprises an outside lipid membrane encapsulating a signal inducing agent (e.g., a non-enzyme or non-protein molecule). In some examples, the nanoparticle is free of any liquid phase (e.g., solid nanoparticles). In other examples, the nanoparticle may comprise a hollow core that contains air or liquid. Such a nanoparticle may be dissociated by ultrasound.

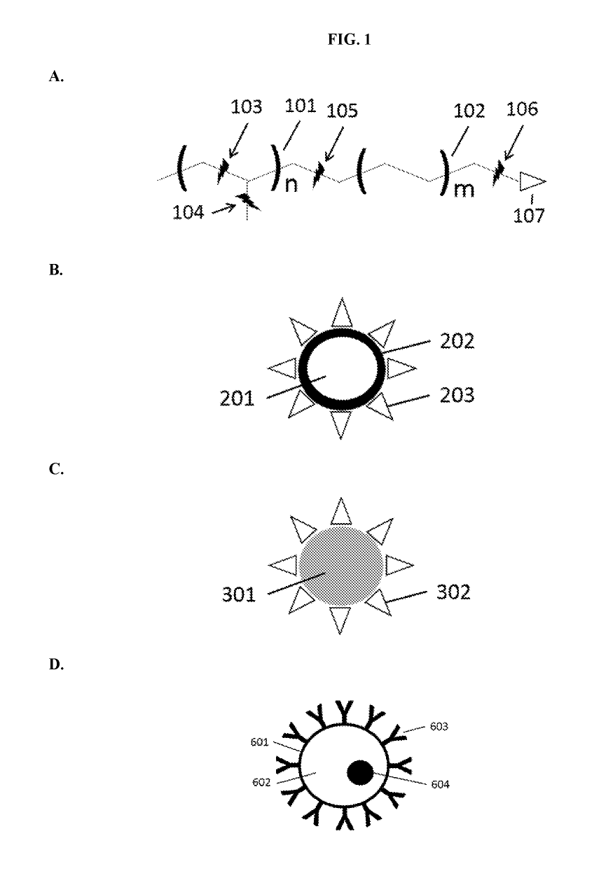

FIGS. 1A-1D illustrate exemplary designs of the nanoparticles described herein.

In one example, the core of the nanoparticles may be polymeric or particulate in nature. Polymers consist of repeating units containing one or more signal inducing agents (101) and may release the signal inducing agents (payloads) by severing pendant (104) and/or backbone (103 and 105) groups. Multiple different signal inducing agents may be contained in a single polymer. Polymers may consist of co-, alt-, branched-, or similar and/or hybrid structures. Structural pieces may contain non-payload elements (102), which may be present for stability or similar functional purposes. Each polymer may be bound to one or more detection species (107). FIG. 1A.

The particles may consist of homo- or heterogeneously distributed payloads. Homogeneous particles (301) may consist of distributions of payload particles in one or more of a polymer, small molecule, and/or crystalline matrix. Heterogeneously distributed payloads may consist of one or more core-shell structures with payload(s) at the core (201) surrounded by one or more of a polymer, small molecule, and/or crystalline shell (202). One or more payloads may be present per particle. Particle surfaces may present one or more detection and/or stability-enhancing species (203 and 302). FIGS. 1B-C.

FIG. 1D illustrates nanoparticles (601) comprising antibodies on the surface (603) as binding agents and having payloads (604) entrapped within the core (602).

Magnetically active particles may be bound to polymers or embedded in particles in order to magnetically address the labels.

In some embodiments, the nanoparticle described herein may further comprise, e.g., a further transition-metal catalysts or a chemiluminopohore or chemiluminophore precursor, which, upon a reaction, produce different detectable signals to those produced by the first transition-metal catalyst. In one example, the nanoparticle contains one signal inducing agent for signal amplification (e.g., a catalyst) and another signal inducing agent that directly releases a signal (e.g., a a chemiluminopohore or chemiluminophore precursor) after being released from the nanoparticle.

The polydispersity index (PDI) of the nanoparticles can be measured by methods known in the art. For example, the measurement of particle size and molecular size can be obtained using, e.g., dynamic light scattering.

### Preparation of Nanoparticles

Nanoparticles described herein can be prepared according to methods known in the art.

In embodiments, the nanoparticles can be prepared by the methods described below and in co-pending application 62/264,782, filed by Applicant, which is incorporated by reference in its entirety.

In embodiments, nanoparticles described herein can be prepared by a method comprising
a. providing a first emulsion comprising an agent of interest, a polymeric matrix, a primary surfactant, and a first solvent system;
b. combining the first emulsion with a second solvent system to create a second emulsion;
c. mixing the second emulsion with a third solvent system to create a nanoparticle suspension; and
d. forming the water-dispersible polymeric nanoparticle in the presence of at least one secondary surfactant.

The agent of interest can be a chemiluminophore or a transition-metal catalyst as described herein. Suitable examples of polymeric matrices, primary surfactants, and solvent systems are described in co-pending application 62/264,782, which is incorporated by reference.

The following represents one example nanoparticle used to illustrate methods of making exemplary nanoparticles useful in the claimed invention. A person of skill in the art would readily identify alternative embodiments in view of the disclosure within that still fall within the scope of the invention.

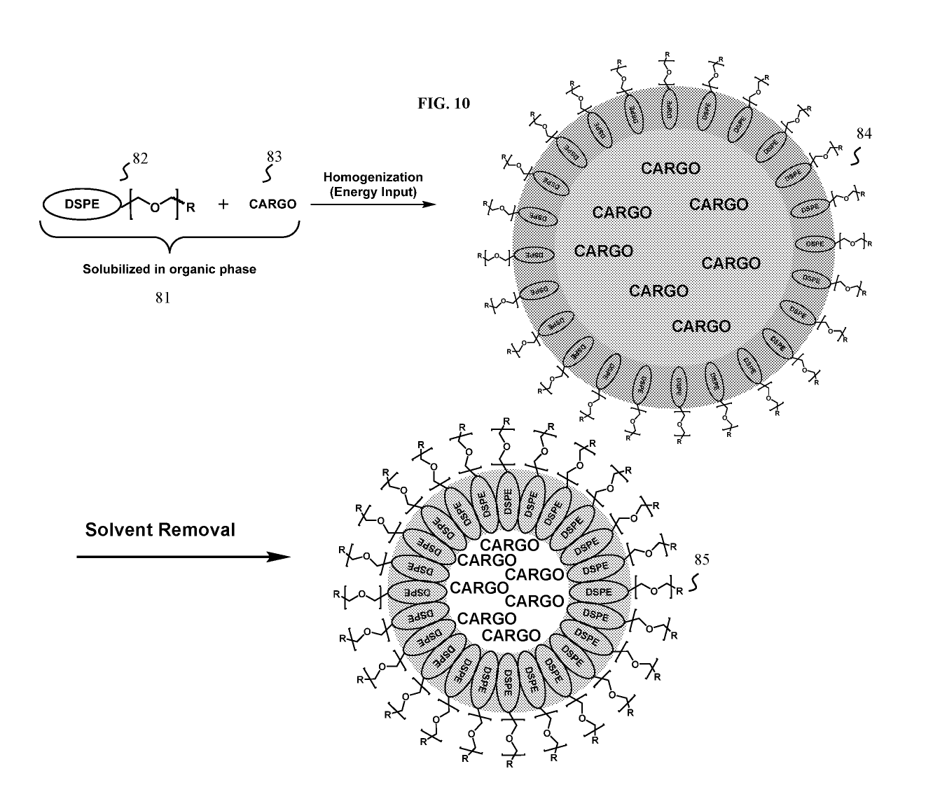

FIG. 10 is a schematic illustrating the synthesis of an example nanoparticle. First, at 81, a polymer DSPE (82) tethered to a functional group (a matrix-forming agent) is solubilized with a group labeled “CARGO” (83) in an organic solvent. The polymer DSPE and the functional groups are described in more detail above. Mixtures of polymers with different functional groups can be used. For example, 33% (or ⅓) of the polymer can have a functional group containing a biotin moiety, and the remaining 66% (or ⅔) can have a free amine moiety. In another example, 66% (or ⅔) of the polymer can have a functional group containing a biotin moiety, and the remaining 33% (or ⅓) can have a free amine moiety. In still another example, 100% of the polymer can have a functional group containing a biotin moiety.

In FIG. 10, CARGO is the material to be encapsulated or embedded into the core structure (e.g., a matrix) of the nanoparticle. For example, CARGO can include a luminophore precursor. CARGO can also include surfactants, such as, for example, PLA. The surfactants can be of different sizes. Addition of surfactants of different sizes (e.g., polymers of different lengths or containing different functional groups) or a mixture of small molecule surfactants (e.g., PLA) and polymeric surfactants increases the stability of the nanoparticles. Once the materials are solubilized in the organic phase, the hydrophobic group of the DSPE surrounds the CARGO at (84). Solvent is subsequently removed yielding the nanoparticle at (85).

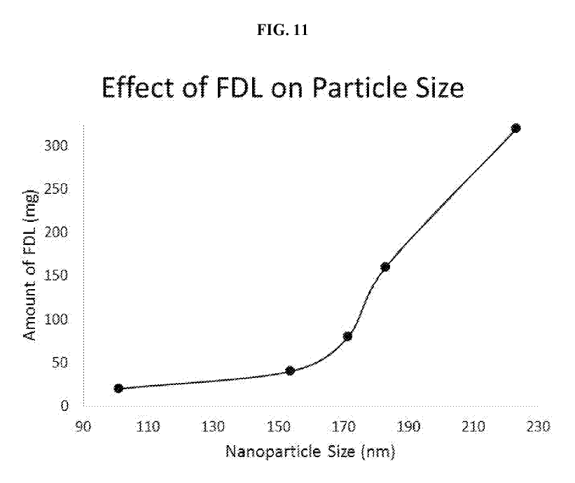

The size of the nanoparticle can be controlled. As seen in FIG. 11, the higher the concentration of fluorescein dilaurate (FDL) present in the nanoparticle, the larger the diameter of the nanoparticle. FIG. 11 is a plot of the data presented in the table below:

| FDL Amount | | |
|---|---|---|
| (mg) | Diameter (nm) | PDI |
| 20 | 98.92 | 0.075 |
| 40 | 153.6 | 0.119 |
| 80 | 179.7 | 0.038 |
| 160 | 196.6 | 0.047 |
| 320 | 228.9 | 0.068 |

The table above and the plot in FIG. 11 illustrate the relationship between particle size and concentration of FDL present in the nanoparticle. These data are indicative of an emulsion formulation process for the nanoparticles. For a given concentration of surfactant(s) and a given energy input, the more “matrix” material present in the starting formulation the larger the resulting nanoparticles will be. The nanoparticle size was measured by dynamic light scattering (DLS).

Accordingly, in one embodiment, the nanoparticle has a diameter between 90 nm and 350 nm. In another embodiment, the nanoparticle has a diameter between 90 nm and 230 nm. In another embodiment, the nanoparticle has a diameter between 150 nm and 200 nm. In another embodiment, the nanoparticle has a diameter between 160 nm and 190 nm. In another embodiment, the nanoparticle has a diameter between 170 nm and 180 nm.

In embodiments, methods described in co-pending application 62/264,782 can overcome existing issues of hydrophilic species encapsulation in polymeric nanoparticles by creating core-shell structures in a two-step emulsification process. This consists first of a “primary” oil-in-oil emulsion. After particles are precipitated through the addition of a third solvent, the resulting mixture is dispersed into a plurality-aqueous phase to form the “secondary” emulsion. Species present in the dispersed phase of the primary emulsion form the core of the resulting particles. Species present in the dispersed organic solvent in the plurality-aqueous emulsion form the shell of the resulting particles.

In the preferred embodiment cyclohexane forms the continuous phase (the first organic solvent) and acetonitrile forms the dispersed phase (the second organic solvent) of the oil-oil emulsion (a first emulsion). The first organic solvent and the second organic solvent are immiscible. The first organic solvent can be, for example, a nonpolar solvent. The second organic solvent can be, for example, a semi-polar solvent.

Poly(maleic anhydride-alt-octadecene) (PMAOD; Sigma-Aldrich) is used as the polymeric surfactant (the primary surfactant). Dissolving PMAOD in cyclohexanes or another suitable solvent (first organic solvent) forms a first solution.

The polymeric matrix is dissolved in the acetonitrile (second organic solvent) and forms the core matrix. Suitable examples of polymeric matrices include, but are not limited to: Poly(L-lactic acid) with diacrylic endcaps (PLLA-DA; 20 kD; PolySciTech); Poly(D,L-lactic acid) with acid endcap (PDLLA-A; 10-15 kD; PolySciTech); Poly(L-lactic acid) with acid endcap (PLLA-A; 15-25 kD; PolySciTech); and Poly(L-lactic acid) with acid endcap (PLLA-A; ˜180 kD; PolySciTech).

An agent of interest (or species to be encapsulated) is also dissolved in the acetonitrile. A suitable example of an agent of interest includes a metal-tetraamidomacrocyclic ligand complex (MTALC; GreenOx Catalysts; U.S. Pat. No. 6,100,394). The PLA-DA-to-MTALC mass ratio is 5:1. The combination of the polymeric matrix and the agent of interest in acetonitrile forms a second solution.

Combining the first solution and the second solution forms a first emulsion. After homogenization of the first emulsion at 7,500 rpm (IKA), benzene (a second solvent system) is added, which clarifies the emulsion, creating a second emulsion. It is important to note that PLA-DA is insoluble in benzene at room temperature.

At least one secondary surfactant may then be added to the second emulsion. Example secondary surfactants include, but are not limited to polymer-surfactant 1,2-distearoyl-sn-glycero-3-phosphoethanolamine-N-[biotinyl(polyethylene glycol)-2000](DSPE-PEG-biotin; Laysan Bio); 1,2-distearoyl-sn-glycero-3-phosphoethanolamine-N-[biotinyl(polyethylene glycol)-3400] (DSPE-PEG-biotin: Lavsan Bio); 1,2-distearoyl-sn-glycero-3-phosphoethanolamine-N-[amine(polyethylene glycol)-2000] (DSPE-PEG-amine; Laysan Bio); and may include either poly(lactic acid) (PLA; 20 kD), or styrene, divinylbenzene, and 2,2′-azosisobutyronitrile (AIBN), or divinylbenzene, pentaerythritol tetrakis(3-mercaptopropionate) (PT3MP; Sigma Aldrich), and 2,2-dimethoxy-2-phenylacetophenone (DMPA; Sigma Aldrich). In an example embodiment, at least one secondary surfactant is capable of undergoing a polymeric reaction to form a polymeric shell.

The second emulsion is then dispersed in water (a third solvent system) with homogenization at 7,500 rpm (IKA), forming a stable emulsion (a nonparticle suspension). The third solvent system comprises a polar solvent such as water. The nanoparticle suspension may be left stirring to evaporate the solvent. The nanoparticle suspension may alternatively have ethanol or a similar solvent added to precipitate particles. The nanoparticle suspension may alternatively be flushed with nitrogen, fitted with a reflux condenser, and heated to 50° C. to enable polymerization of the at least one secondary surfactant to form a polymeric shell. The nanoparticle suspension may alternatively be irradiated with long wave UV to facilitate a thiolene-click gelation reaction. The nanoparticle suspension may alternatively be added to excess water in a secondary step to fix the nanoparticle size.

By controlling the shell properties, this process enables control over the encapsulation and release of hydrophilic species. Through the presence of an aqueous component and one or more stabilizers in the dispersed organic phase of the primary emulsion proteins may be encapsulated with this approach.

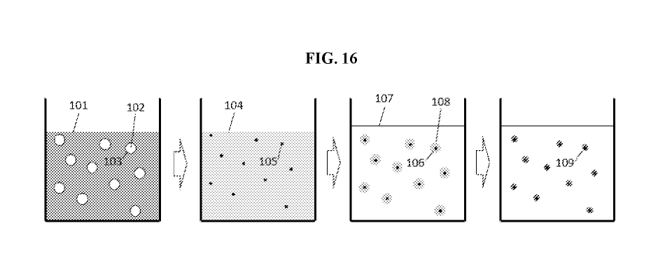

FIG. 16 shows a schematic of the nanoparticle fabrication methods. The primary emulsion is created with two immiscible oils (101 and 102) stabilized with a polymeric surfactant (103). The dispersed phase (102) contains one or more species to be encapsulated, one or more polymeric matrix-forming elements, and any stabilizers. Upon addition of a suitable solvent that dissolves compounds 101 and 102, the emulsion is clarified (104) and the particle cores (105) are precipitated. The clarified organic solution containing precipitated particles is then dispersed (106) into a plurality-aqueous phase (107). This dispersion is stabilized through the presence of one or more solvents containing one or more aqueous-soluble regions (108). The added shell-forming agents then create a shell (109) around the particle cores either directly and/or through polymerization reactions. Organic solvents are then removed by dissolution, evaporation, and/or filtration to produce the final, aqueous-soluble particles.

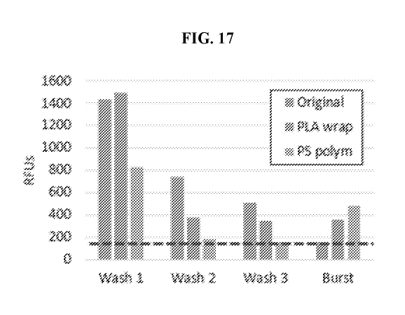

FIG. 17 demonstrates the tuning of the shell region to enable effective encapsulation of a water-soluble salt, the metal-tetraamidomacrocyclic ligand complex (MTALC; GreenOx Catalysts; U.S. Pat. No. 6,100,394). The “original” formulation contained only the polymer-surfactant 1,2-distearoyl-sn-glycero-3-phosphoethanolamine-N-[biotinyl(polyethylene glycol)-2000] (DSPE-PEG-biotin; Laysan Bio) as a shell-forming agent during the aqueous emulsion. The “PLA wrap” formulation contained a 20 kD poly(lactic acid) (PLA; PolymerSourcelnc) as a shell-forming agent during the aqueous emulsion. The “PS polym” formulation comprised styrene, divinylbenzene, and 2,2′-azosisobutyronitrile (AIBN) as shell-forming agents during the aqueous emulsion. After emulsion formation, the flask was flushed with nitrogen, fitted with a reflux condenser, and heated to 50° C. to allow polymerization.

Particles were dialyzed into 1×PBS and loaded into a microfuge spin-filter tube with a 20 kD membrane. Samples were spun and the filtrate was collected and tested for MTALC activity according to the procedure above. Each “wash” consists of particle resuspension into an addition of an equal amount of PBST and subsequent centrifugation and filtrate collection. Particles were “burst” using acetone followed by the addition of sodium bicarbonate buffer at pH 10. The baseline fluorescence reading is shown as a dashed line and labeled “baseline.”

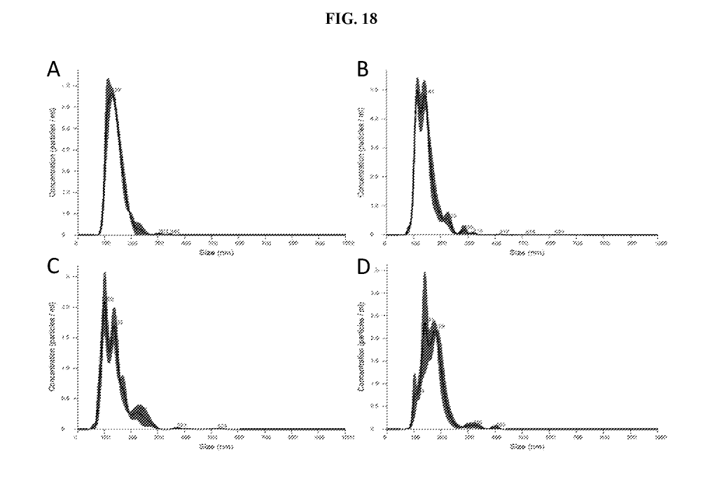

FIGS. 18A-D show representative data obtained from Nanosight measurements and demonstrates the control that can be exhibited over the various nanoparticle structures. This ability to control the size and relative dispersity of the formed particles can be seen for all formulations including FIG. 18A) example 6.5, FIG. 18B) from example 6.6, FIG. 18C) example 6.7, and FIG. 18D) example 6.8. This ability to control particle size through multiple emulsification steps demonstrates the robustness of the developed formulation method.

### III. BINDING AGENT

The nanoparticle described herein is conjugated to a binding agent, i.e., a molecule that binds to an analyte of interest. The binding agents include, but not limited to, antibodies, enzymes, oligonucleotides, DNA, RNA, PNA, or LNA, proteins, peptides, polypeptides, receptors, ligands, small molecules, aptamers, polysaccharides, plastibodies, or any selective detection materials disclosed herein. The ratio of the binding agent present on the nanoparticle, either in the polymer or on the particle surface, to the signal inducing agents may be tuned to optimize detection by conventional methods.

In some embodiments, the binding agent can be an antibody specific to the analyte, a nucleic acid, which can be a single-strand DNA or RNA, or an aptamer. Alternatively, the binding agent can be a member of a receptor/ligand pair. Selection of a suitable binding agent would depend on the nature of the analyte of interest to be detected in the assay method described herein. For example, if the analyte is a nucleic acid, a nucleic acid having a sequence complementary to the target nucleic acid may be used as the binding agent. Alternatively, if the analyte of interest is a member of a receptor/ligand pair, the other member of the same receptor ligand pair may be used as the binding agent. A receptor/ligand pair can be any two binding partners that have specific binding activity to each other, for example, biotin/streptavidin.

In some examples, the binding agent specifically binds to the analyte. An binding agent that “specifically binds” (used interchangeably herein) to a target or an epitope thereof is a term well understood in the art, and methods to determine such specific binding are also well known in the art. An agent is said to exhibit “specific binding” if it reacts or associates more frequently, more rapidly, with greater duration and/or with greater affinity with a particular target analyte than it does with alternative targets. A binding agent “specifically binds” to a target analyte if it binds with greater affinity, avidity, more readily, and/or with greater duration than it binds to other substances. It is also understood by reading this definition that, for example, an agent that specifically binds to a first target analyte may or may not specifically or preferentially bind to a second target analyte. As such, “specific binding” or “preferential binding” does not necessarily require (although it can include) exclusive binding. Generally, but not necessarily, reference to binding means preferential binding.

In some embodiments, the binding agent is an antibody that binds to the analyte of interest. An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

In some instances, the binding agent is modified by a molecule that allows for the attachment of the binding agent onto the nanoparticle. For example, the binding agent may be conjugated to biotin. Via biotin-streptavidin interaction, the biotinylated binding agent can be attached to the nanoparticle.

### IV. TRANSITION METAL CATALYSTS

The transition-metal catalysts described herein are substances that increase the rate of a chemical reaction without itself undergoing any permanent chemical change, so as to covert a suitable substrate to a product, wherein the conversion results in a signal change. In some instances, the conversion leads to presence of increase of a detectable signal, e.g., the product releases a signal while the substrate does not. In other cases, the conversion leads to the diminish or reduction of a signal, e.g., the substrate releases a signal while the product does not.

In some embodiments, the transition-metal catalysts described herein are precursors to the catalytically active species in a reaction.

In some embodiments, the transition-metal catalyst is a metalorganic compound, which is a complex comprising a metal core (e.g., Fe, Mg, Cu, Mn, Pd, Pt, Ag, Ru, or Ce) and one or more organic ligands, e.g., porphyrin, substituted porphyrins, bipyridyls, bis-diimines, polydentates, ethanediamines, ethylenediamines, pentaaminecarbonatos, tetraaminecarbonatos, coumarins. Specific examples include, but are not limited to, iron porphyrins, hemin, ruthenium diimines, ruthenium bipyridyls, iridium-coumarin complexes, bis(1,2-ethanediamine)copper, nickel porphyrin, and/or calcium ethylenediamine tetraacetate.

In some examples, the transition-metal catalyst is a reactive oxygen species generator, which catalyzes a chemical reaction to produce reactive oxygen species (ROS), i.e., chemically active molecules containing oxygen. Such catalysts can be radicals by ions and molecules, including, but not limited to, Fe(II), Fe(III), Ce(III), Ce(IV), Cu(I), Cu(II), Cr(III), Cr(VI), Co(II), Co(III), Ru, Al(0), Al(III), or a metalorganic compound as described herein. The reaction mixture containing this type of catalyst may contain a suitable substrate, which can be converted to a product by the catalyst, leading to a signal change, e.g., fluorescence increase or decrease or absorbance increase or decrease. Exemplary substrates include, but are not limited to, resazurin, coumarin-3-carboxylic acid, fluorescein, methyl orange, terepthalic acid, sodium terepthalate, 2-[6-(4′-hydroxy)phenoxy-3H-xanthen-3-on-9-yl]benzoic acid, 2-[6-(4′-amino)phenoxy-3H-xanthen-3-on-9-yl]benzoic acid, fluorescein, 2′,7′-dichlorofluorescein, 2,7-dichlorodihydrofluorescein, hydroethidine, 1,3-diphenylisobenzofuran, 2-(2-pyridil)-benzothiazoline, 4-(9-anthroyloxy)-2,2,6,6-tetramethylpiperidine-1-oxyl, 1,3-cyclohexanedione, coumarin-3-carboxylic acid NHS ester, cis-parinaric acid, 4,4-difluoro-5-(4-phenyl-1,3-butadienyl)-4-bora-3a,4a-diaza-s-indacene-3-undecanoic acid), dipyridamole, diphenyl-1-pyrenylphosphine, 2,7-dichlorodihydrofluorescein diacetate, or TEMPO. Additional components may be included in the reaction mixture to enhance the signal change, including, for example, redox-active fluorophore, redox-active absorber, or H₂O₂. The chemical reaction catalyzed by the catalyst can take place in a solution, which may be aqueous with possible organic cosolvents. The pH of the solution may be tuned for optimal detection. The reaction may need heat to increase reaction rate (e.g., to a point that does not degrade H₂O₂) or light to increase reaction rate.

In other embodiments, the catalyst is a singlet oxygen generator, which is a substance that produces singlet oxygen (dioxidene and dioxygen), an inorganic chemical in an excited state, via a chemical reaction. Examples include, but are not limited to, photosensitizers (see www3.nd.edu/˜ndrlrcdc/Compilations/QY/QY1.HTM), phthalocyanines (metal-free or with any metal core), porphryins (metal-free or with any metal core), methylene blue, or rose Bengal. Substrates for this type of catalysts include singlet oxygen-reactive fluorophores, absorbers, chemiluminophores, or photosensitizers. Examples are 9,10-dimethylanthracene, 1,3-diphenylisobenzofuran, 9-[2-(3-carboxy-9,10-dimethyl)anthryl]-6-hydroxy-3H-xanthen-3-one, or 9-[2-(3-carboxy-9,10-diphenyl)anthryl]-6-hydroxy-3H-xanthen-3-one. The reaction catalyzed by the catalyst may take place in a solution (e.g., an aqueous solution) which may contain EtOH, IPA, DMF, DMSO, or a combination thereof. In some instances, DMSO may be required. To enhance signal detection, the reaction mixture may further comprise dissolved oxygen (or source), singlet oxygen-reactive fluorescent or absorbent species, and/or DMSO in some cases. An energy source such as light or thermal may be needed for the reaction, for example, light-induced singlet oxygen generation.

In still other embodiments, the transition-metal catalyst can effect an reaction on a chemiluminscent precursor to yield a chemiluminscent compound (e.g., the transition metal catalyst can affect the cleavage of a fluorescence quenching group attached to the chemiluminscent precursor. Exemplary transition metal complexes for such reactions are described herein. Suitable examples of transition metal complexes can be found in U.S. Pat. Nos. 6,100,394; 8,722,881; and 8,754,206, all of which are incorporated by reference.

In an example embodiment, the transition metal catalyst is represented by structural formula I,

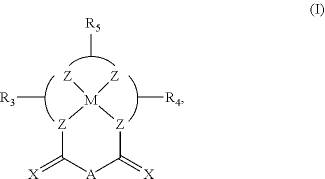

or an oxidized or reduced form thereof, wherein
M is a metal;
A is —CR₁R₂— or —NR_{1′}—;
   wherein when A is —CR₁R₂—, R₁ and R₂ are the same or different, linked or nonlinked, and each is selected from the group consisting of substituents which are unreactive, form strong bonds intramolecularly within said R₁ and R₂ and with the carbon C to which they are bound, are sterically hindered and are conformationally hindered such that oxidative degradation of a metal complex of the compound is restricted when the complex is in the presence of an oxidizing medium; and
   wherein when A is —NR_{1′}—, R_{1′} is C₁₋₂₀ alkyl, C₂₋₂₀ alkenyl, C₂₋₂₀ alkynyl, or phenyl;
   Z is a metal complexing atom selected from the group consisting of N, NH, and O;
   X is a functionality;
   wherein both Z and X are resistant to oxidative degradation such that each confers resistance to oxidative degradation to the metal complex of the compound when the complex is in the presence of an oxidizing medium;
   R₃ is a unit joining the adjacent Z atoms selected from the group consisting of:

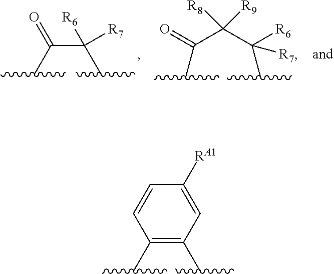

wherein R₆, R₇, R₈ and R₉ pairwise and cumulatively are the same or different and each is selected from the group consisting of hydrogen, C₁₋₂₀ alkyl, C₂₋₂₀ alkenyl, C₂₋₂₀ alkynyl, C₆₋₁₀ aryl, and halogen; or any pair of R₆, R₇, R₈ and R₉ can, together with the atoms to which they are attached, form a C₄₋₁₀ cycloalkyl;
R^{A1} is hydrogen, halogen, or —X¹—Y¹—Z¹, wherein
   X¹ is —C(R^{X1})₂—, —C(O)—, —C(O)O—, —C(O)NH—, —CR^{X1}═CR^{X1}—, —NR^{X1}—, —NR^{X1}C(O)—, —O—, or —OC(O)—, wherein R^{X1} is hydrogen or C₁₋₆ alkyl;
   Y¹ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y¹ are optionally and independently replaced by -Cy¹-,
—NR^{Y1}—, —N(R^{Y1})C(O)—, —C(O)N(R^{Y1})—, —O—, —C(O)—, —OC(O)—, —C(O)O—, or —N═N—, wherein R^{Y1} is hydrogen or C₁₋₆ alkyl; and
   each Cy¹ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
   Z¹ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
R₄ is a unit joining the adjacent Z atoms comprised of

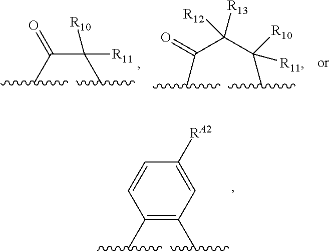

wherein R₁₀, R₁₁, R₁₂ and R₁₃ pairwise and cumulatively are the same or different and each is selected from the group consisting of hydrogen, C₁₋₂₀ alkyl, C₂₋₂₀ alkenyl, C₂₋₂₀ alkynyl, C₆₋₁₀ aryl, and halogen; or any pair of R₁₀, R₁₁, R₁₂ and R₁₃ can, together with the atoms to which they are attached, form a C₄₋₁₀ cycloalkyl;
   R^{A2} is hydrogen, halogen, or —X²—Y²—Z², wherein
      X² is —C(R^{X2})₂—, —C(O)—, —C(O)O—, —C(O)NH—, —CR^{X2}═CR^{X2-}, —NR^{X2}, —NR^{X2}C(O)—, —O—, or —OC(O)—, wherein R^{X2} is hydrogen or C₁₋₆ alkyl;
      Y² is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y² are optionally and independently replaced by -Cy²-,
   —NR^{Y2}—, —N(R^{Y2})C(O)—, —C(O)N(R^{Y2})—, —O—, —C(O)—, —OC(O)—, —C(O)O—, or —N═N—, wherein R^{Y2} is hydrogen or C₁₋₆ alkyl; and
      each Cy² is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
      Z² is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl
R₅ is a unit joining adjacent Z atoms selected from the group consisting of
   (i)

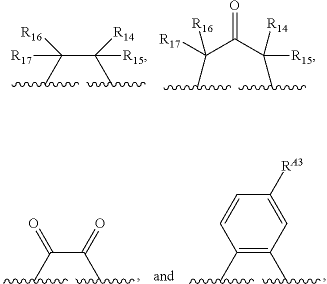

wherein R₁₄, R₁₅, R₁₆ and R₁₇ are the same or different and each is hydrogen, C₁₋₂₀ alkyl, C₂₋₂₀ alkenyl, C₂₋₂₀ alkynyl, C₆₋₁₀ aryl, and halogen;
or any pair of R₁₄, R₁₅, R₁₆ and R₁₇ can, together with the atoms to which they are attached, form a C₄₋₁₀ cycloalkyl;
R^{A3} is hydrogen, halogen, or —X³—Y³—Z³, wherein
   X³ is —C(R^{X3})₂—, —C(O)—, —C(O)O—, —C(O)NH—, —CR^{X3}═CR^{X3}—, —NR^{X3}, —NR^{X3}C(O)—, —O—, or —OC(O)—, wherein R^{X3} is hydrogen or C₁₋₆ alkyl;
   Y³ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y³ are optionally and independently replaced by -Cy³-,
—NR^{Y3}—, —N(R^{Y3})C(O)—, —C(O)N(R^{Y3})—, —O—, —C(O)—, —OC(O)—, —C(O)O—, or —N═N—, wherein R^{Y3} is hydrogen or C₁₋₆ alkyl; and
   each Cy³ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
   Z³ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
optionally a counter ion selected from H₂O, ammonium, and halogen.

In embodiments, a transition-metal catalyst comprises a structure according to formula I′,

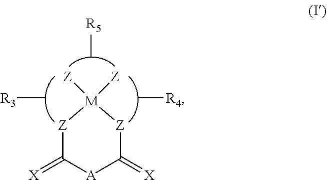

or an oxidized or reduced form thereof, wherein
M is a metal;
A is —CR₁R₂— or —NR_{1′}—;
   wherein when A is —CR₁R₂—, R₁ and R₂ are the same or different, linked or nonlinked, and each is selected from the group consisting of substituents which are unreactive, form strong bonds intramolecularly within said R₁ and R₂ and with the carbon C to which they are bound, are sterically hindered and are conformationally hindered such that oxidative degradation of a metal complex of the compound is restricted when the complex is in the presence of an oxidizing medium; and
   wherein when A is —NR_{1′}—, R_{1′} is C₁₋₂₀ alkyl, C₂₋₂₀ alkenyl, C₂₋₂₀ alkynyl, or phenyl;
   Z is a metal complexing atom selected from the group consisting of N, NH, and O;
   X is a functionality;
   wherein both Z and X are resistant to oxidative degradation such that each confers resistance to oxidative degradation to the metal complex of the compound when the complex is in the presence of an oxidizing medium;
   R₃ is a unit joining the adjacent Z atoms comprised of

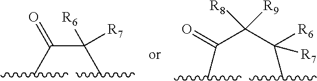

wherein R₆, R₇, R₈ and R₉ pairwise and cumulatively are the same or different and each is selected from the group consisting of hydrogen, C₁₋₂₀ alkyl (e.g., halogenated C₁₋₂₀ alkyls such as —CF₃), C₂₋₂₀ alkenyl, C₂₋₂₀ alkynyl, C₆₋₁₀ aryl (e.g., halogenated C₆₋₁₀ aryl), and halogen, or R₆ and R₇, or R₈ and R₉, combine to form a 3-10 membered cycloaliphatic; and
R₄ is a unit joining the adjacent Z atoms comprised of

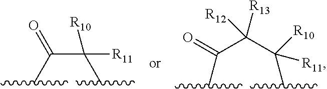

wherein R₁₀, R₁₁, R₁₂ and R₁₃ pairwise and cumulatively are the same or different and each is selected from the group consisting of hydrogen, C₁₋₂₀ alkyl (e.g., halogenated C₁₋₂₀ alkyls such as —CF₃), C₂₋₂₀ alkenyl, C₂₋₂₀ alkynyl, C₆₋₁₀ aryl (e.g., halogenated C₆₋₁₀ aryl), and halogen, or R₁₀ and R₁₁, or R₁₂ and R₁₃, combine to form a 3-10 membered cycloaliphatic;
R₅ is a unit joining adjacent Z atoms selected from the group consisting of
   (i)

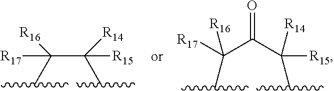

wherein R₁₄, R₁₅, R₁₆ and R₁₇ are the same or different and each is hydrogen, C₁₋₂₀ alkyl (e.g., halogenated C₁₋₂₀ alkyls such as —CF₃), C₂₋₂₀ alkenyl, C₂₋₂₀ alkynyl, C₆₋₁₀ aryl (e.g., halogenated C₆₋₁₀ aryl), and halogen, or R₁₄ and R₁₅, or R₁₆ and R₁₇, combine to form a 3-10 membered cycloaliphatic; and
(ii) optionally-substituted aryl or heteroaryl groups.

In embodiments, a transition-metal catalyst is represented by the structure of any one of formula II, formula IIA, formula IIB, formula IIIA, formula IIIB, formula IVA, formula IVB, or formula V as described herein.

In some embodiments, M is a group 6, 7, 8, 9, 10, or 11 metal.

In other embodiments, M is Cr, Mn, Fe, Co, Ni, or Cu.

In other embodiments, M is Fe (e.g., Fe(II) or Fe(III)).

In another embodiment the transition metal catalyst is selected from:

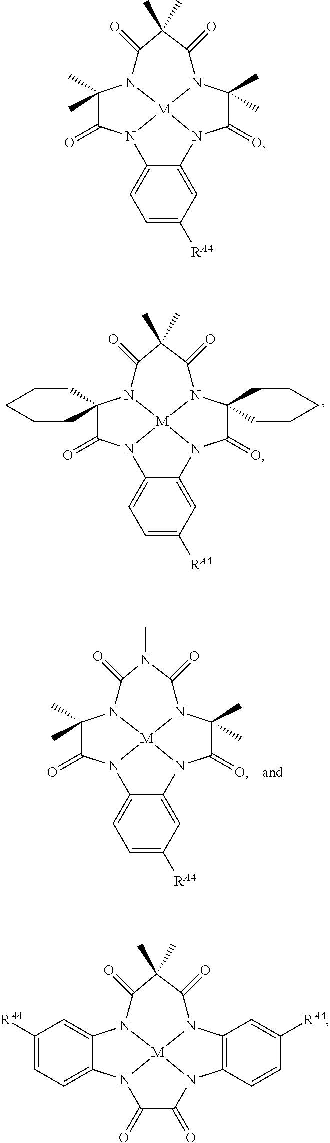

wherein
   M is a metal selected from Fe, Mg, Cu, Mn, Pd, Pt, Ag, Ru, and Ce; and
   R^{A4} is hydrogen, halogen, or —X⁴—Y⁴—Z⁴, wherein
   X⁴ is —C(R^{X4})₂—, —C(O)—, —C(O)O—, —C(O)NH—, —CR^{X4}═CR^{X4}—, —NR^{X4}—, —NR^{X4}C(O)—, —O—, or —OC(O)—, wherein R^{X4} is hydrogen or C₁₋₆ alkyl;
   Y⁴ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y⁴ are optionally and independently replaced by -Cy⁴-, —NR^{Y4}—, —N(R^{Y4})C(O)—, —C(O)N(R^{Y4})—, —O—, —C(O)—, —OC(O)—, —C(O)O—, or —N═N—, wherein R^{Y4} is hydrogen or C₁₋₆ alkyl; and
   each Cy⁴ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
   Z⁴ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
a suitable counter ion selected from H₂O and halogen.

In other embodiments, M is Fe (e.g., Fe(II) or Fe(III)).

In other embodiments, R^{A1} is hydrogen. In other embodiments, R^{A1} is halogen selected from F, Cl, Br, and I.

In other embodiments, R^{A4} is hydrogen. In other embodiments, R^{A4} is halogen selected from F, Cl, Br, and I.

In other embodiments, Y¹, Y, Y³, or Y⁴ is a bivalent linker comprising two or more repeating units of ethylene glycol (poly(ethylene glycol)). In other embodiments, the two or more repeating units of ethylene glycol can be diethylene glycol, triethylene glycol, tetraethylene glycol, or hexylene glycol.

In example embodiments, Z¹, Z², Z³, or Z⁴ is an α,β unsaturated enone or ynone represented by the following structural formulas:

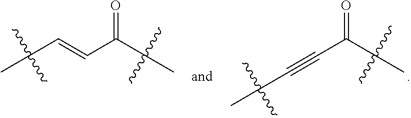 [SMILES: */C=C/C(*)=O ; *C#CC(*)=O]

In other example embodiments, Z¹, Z², Z³, or Z⁴ is a Michael acceptor.

The transition metal catalysts can be prepared according to conditions analogous to those found in U.S. Pat. Nos. 6,100,394; 8,722,881; and 8,754,206. The process for synthesizing the transition metal catalysts is described in the schemes below:

Schemes 1A-B describe the synthesis of base ligands S1.8 and S1.11 and their corresponding metal (III) complexes.

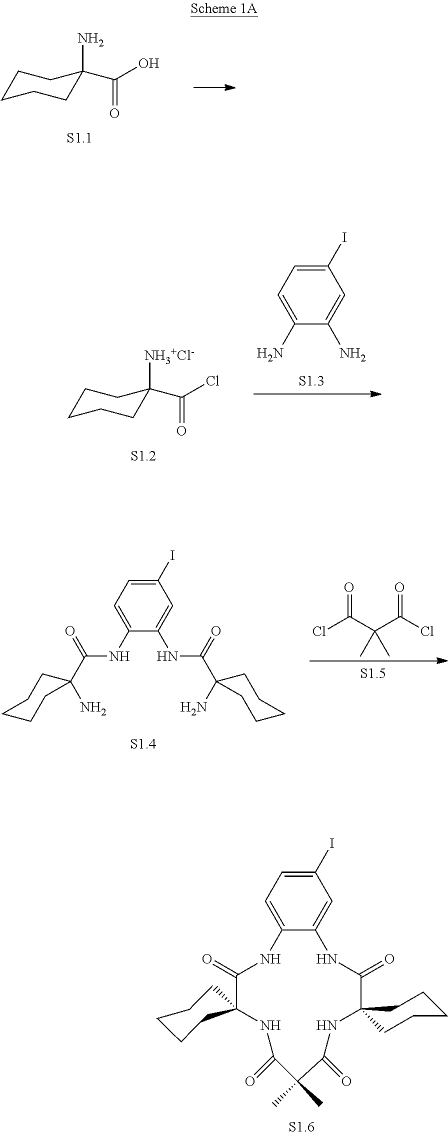

In Scheme 1A, starting material S1.1 is acidified with thionyl chloride to form S1.2. Compound S1.2 is exposed to S1.3 in the presence of a base to form compound S1.4. A base is added to a mixture containing S1.4 and S1.5 is added, yielding product S1.6.

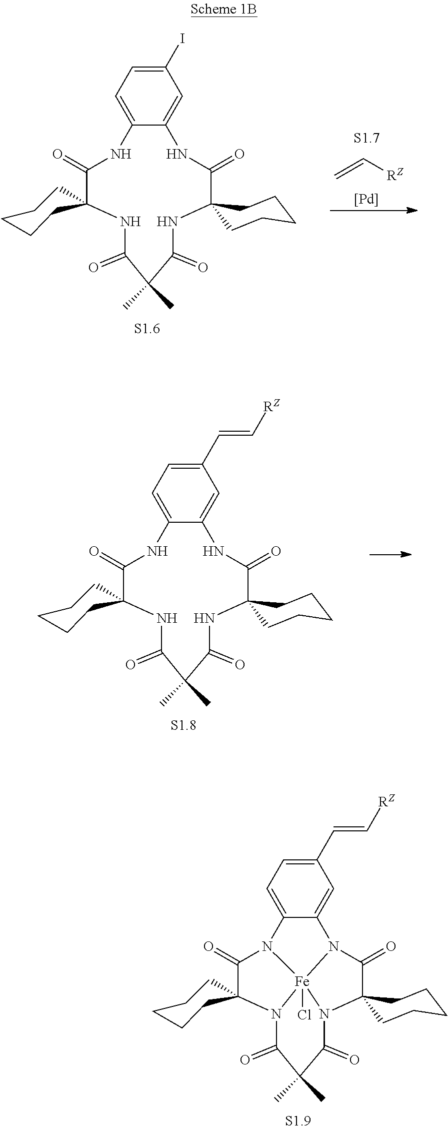

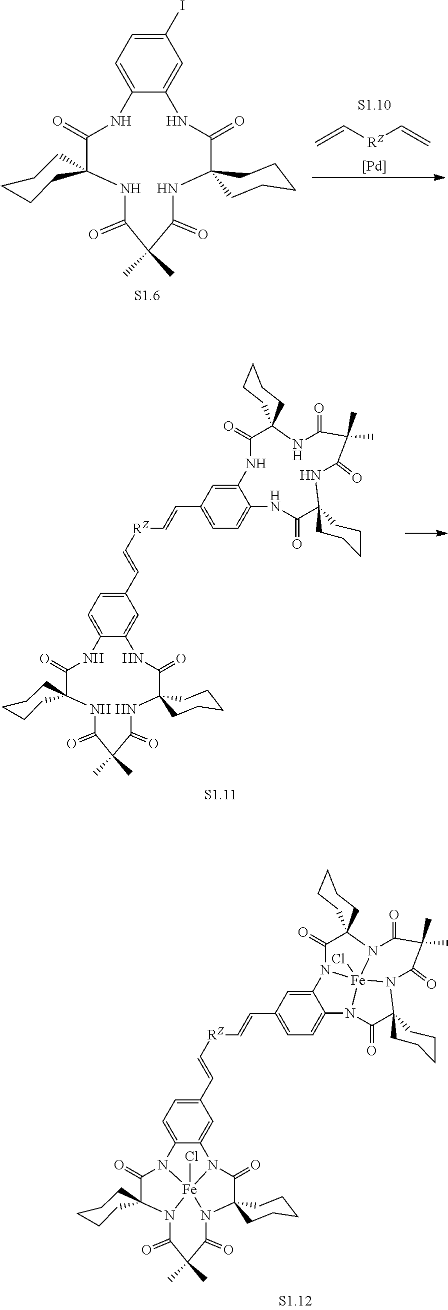

In Scheme 1B, compound S1.6 is reacted with S1.7 under Heck conditions (in the presence of a base and a suitable palladium catalyst) to form compound S1.8. A metal is complexed to compound S1.8 under conditions analogous to those described in U.S. Pat. Nos. 6,100,394 and 8,754,206. For example, S1.8 is dissolved in dry THF, cooled to 0° C., and to this mixture, n-BuLi is added under argon, followed by addition of FeCl₂ to give S1.9.

Alternatively, as illustrated in Scheme 1B, compound S1.6 can be reacted with S1.10 under Heck conditions to form compound S1.11. A metal can then be complexed as described above to form compound S1.12.

Scheme 2A-B describe the synthesis of base ligands S2.8 and S2.11 and their corresponding metal (III) complexes.

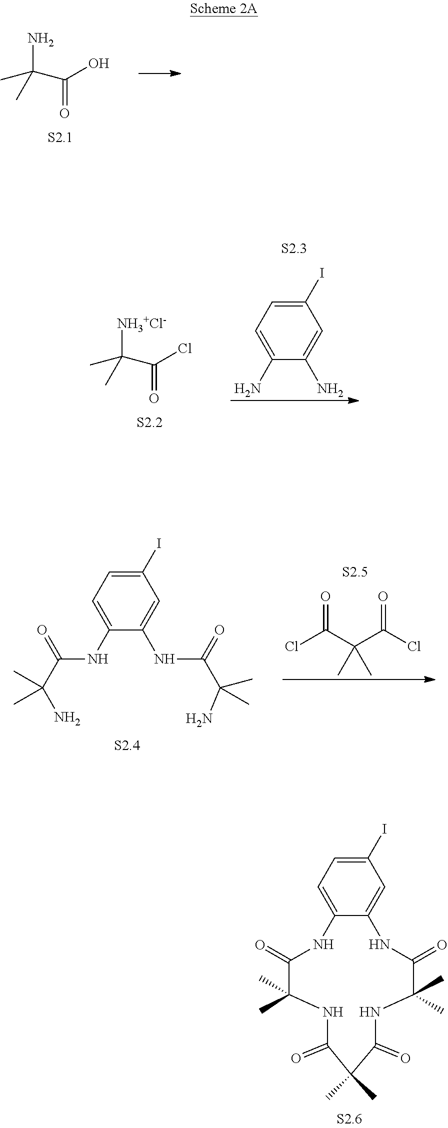

In Scheme 2A, starting material S2.1 is acidified with thionyl chloride to form S2.2. Compound S2.2 is exposed to S2.3 in the presence of a base to form compound S2.4. A base is added to a mixture containing S2.4 and S2.5 is added, yielding product S2.6.

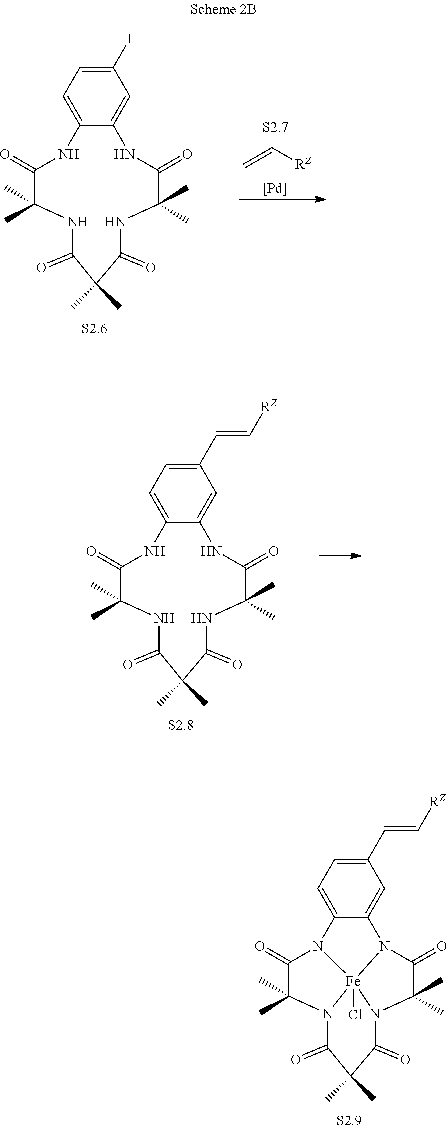

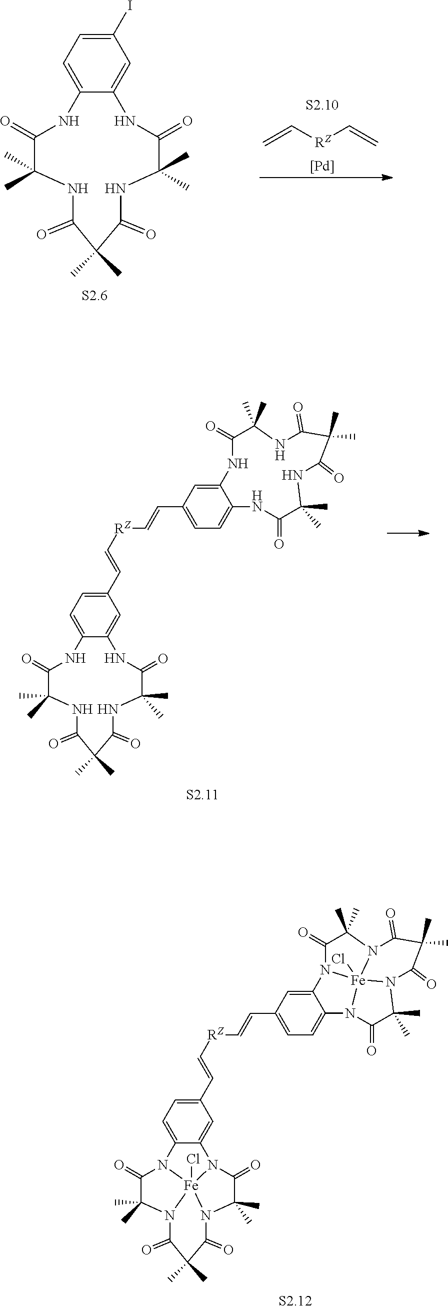

In Scheme 2B, compound S2.6 is reacted with S2.7 under Heck conditions (in the presence of a base and a suitable palladium catalyst) to form compound S2.8. A metal is complexed to compound S2.8 under conditions analogous to those described in U.S. Pat. Nos. 6,100,394 and 8,754,206. For example, S2.8 is dissolved in dry THF, cooled to 0° C., and to this mixture, n-BuLi is added under argon, followed by addition of FeCl₂ to give S2.9.

Alternatively, as illustrated in Scheme 2B, compound S2.6 can be reacted with S2.10 under Heck conditions to form compound S2.11. A metal can then be complexed as described above to form compound S2.12.

Scheme 3A-B describe the synthesis of base ligands S3.9 and S3.12 and their corresponding metal (III) complexes.

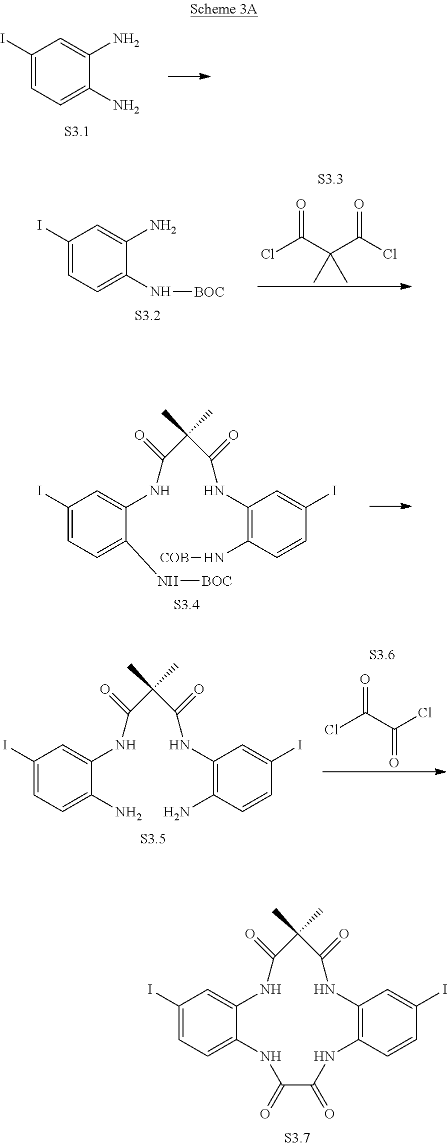

In Scheme 3A starting material 3.1 is protected by tert-butyl carbonate (BOC) by exposing compound 3.1 to di-tert-butyl dicarbonate in the presence of a base to create compound S3.2. Compound 3.2 is exposed to compound S3.3 in the presence of a base to give compound S3.4. The BOC group of compound S3.4 is removed to give compound S3.5 via exposure of compound S3.4 to an acid. Compound S3.5 is treated with a base and exposed to S3.6 to give compound S3.7.

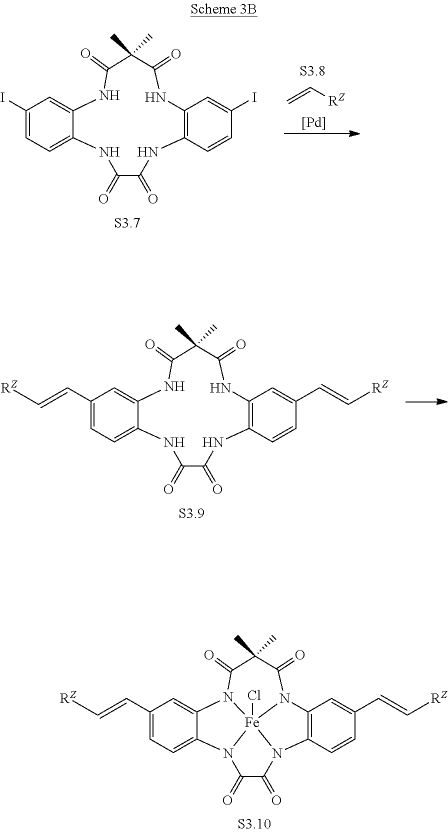

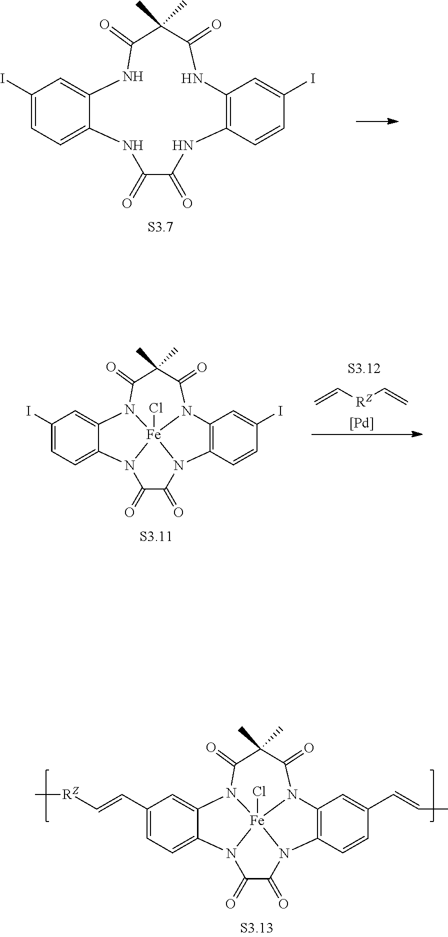

In Scheme 3B, compound S3.7 is reacted with S3.8 under Heck conditions (in the presence of a base and a suitable palladium catalyst) to form compound S3.9. A metal is complexed to compound S3.9 under conditions analogous to those described in U.S. Pat. Nos. 6,100,394 and 8,754,206. For example, S3.9 is dissolved in dry THF, cooled to 0° C., and to this mixture, n-BuLi is added under argon, followed by addition of FeCl₂ to give S3.10.

Alternatively, as illustrated in Scheme 3B, compound S3.7 can be complexed to a metal as described above to form S3.11, Compound S3.11 can be reacted with S3.12 under Heck conditions to form compound S3.13.

In Scheme 4, the functionalized transition metal catalyst (S4.2) can subsequently be reacted with compound S4.1 to give compound S4.3.

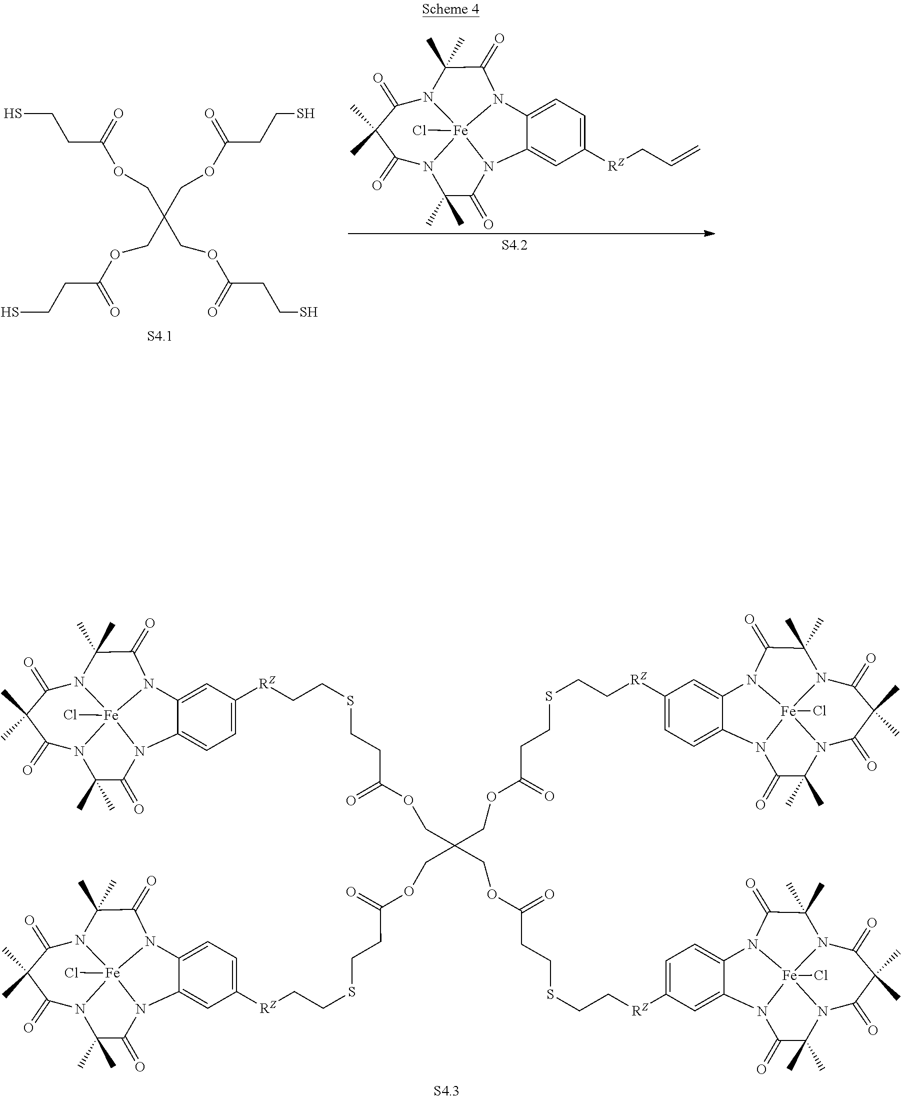

In Scheme 5, the functionalized transition metal catalyst (S5.3) can subsequently be reacted with compounds S5.1 and S5.2 to give compound S5.4.

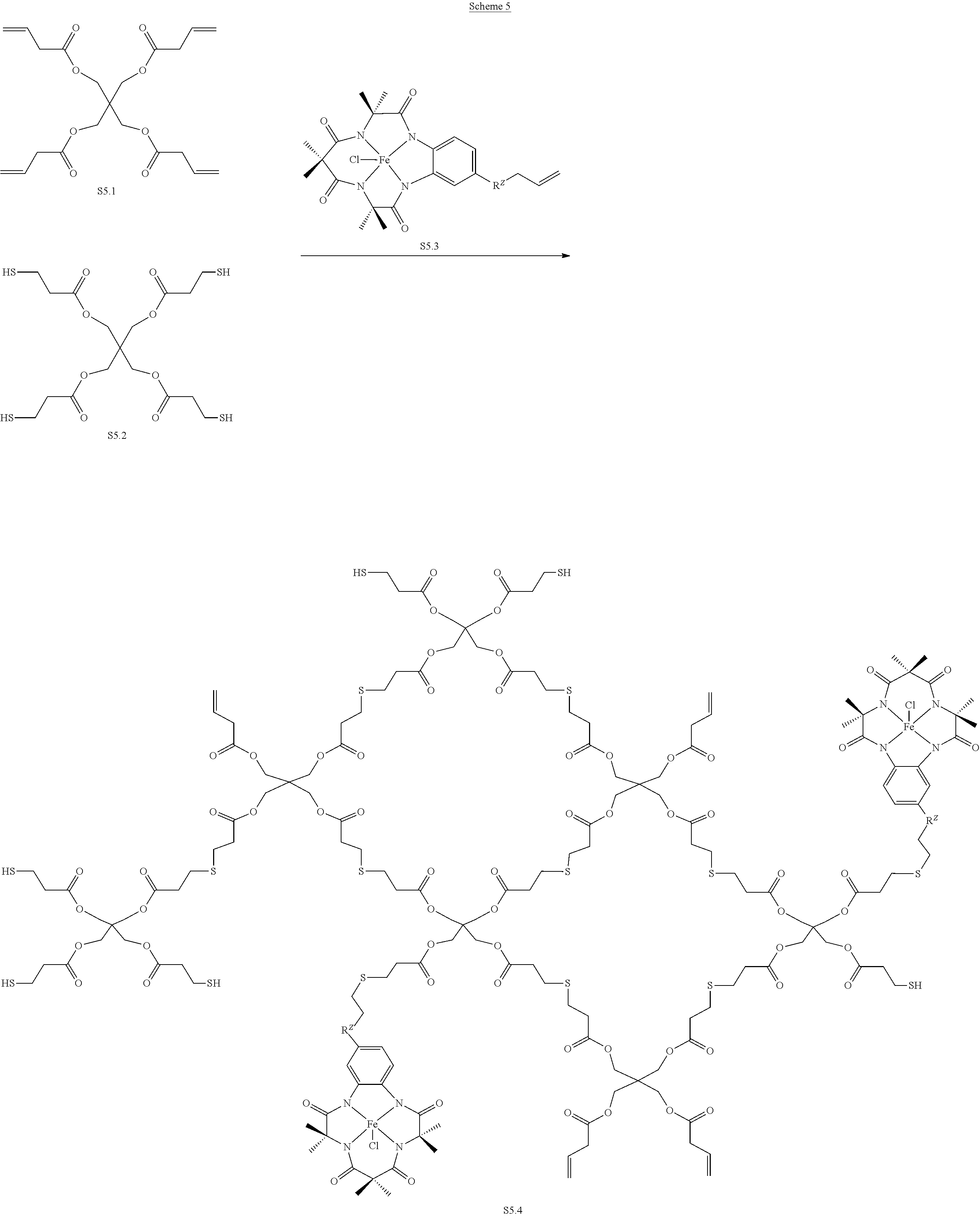

In Scheme 6, the functionalized transition metal catalyst (S6.3) can subsequently be reacted with compounds S6.1 and S6.2 to give compound S6.4.

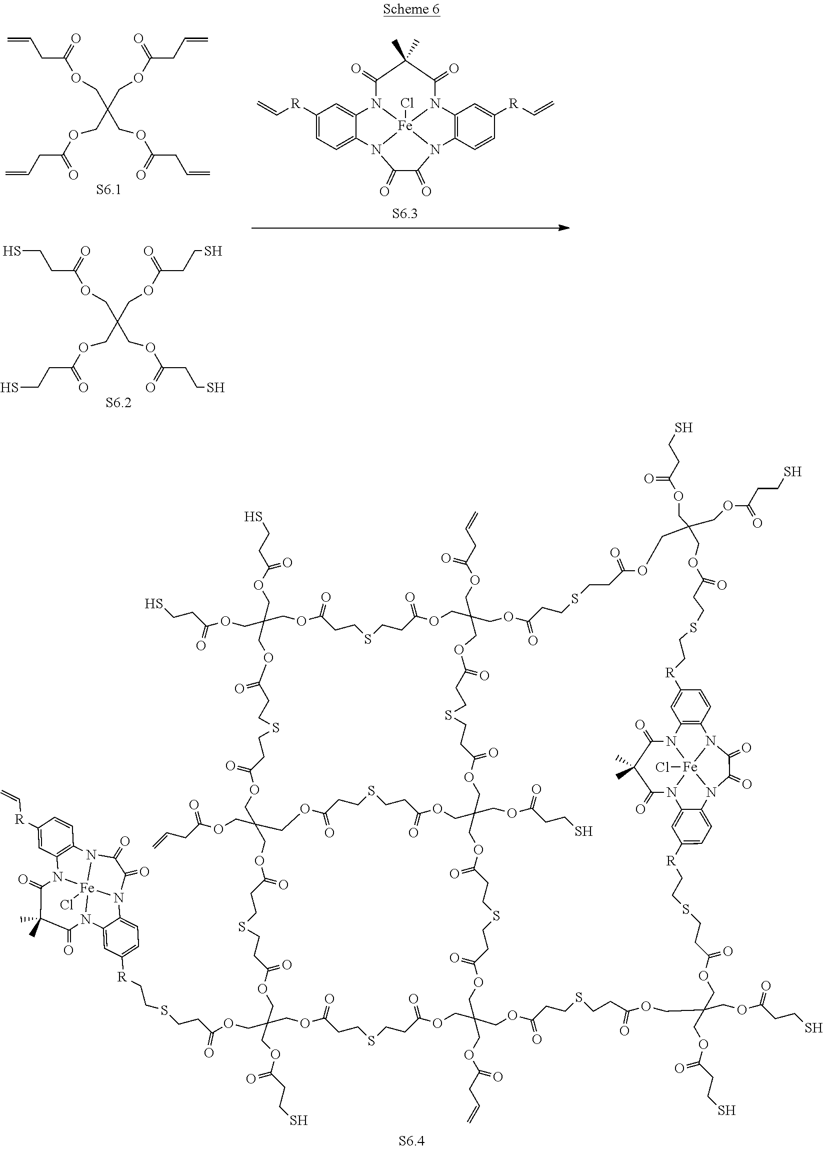

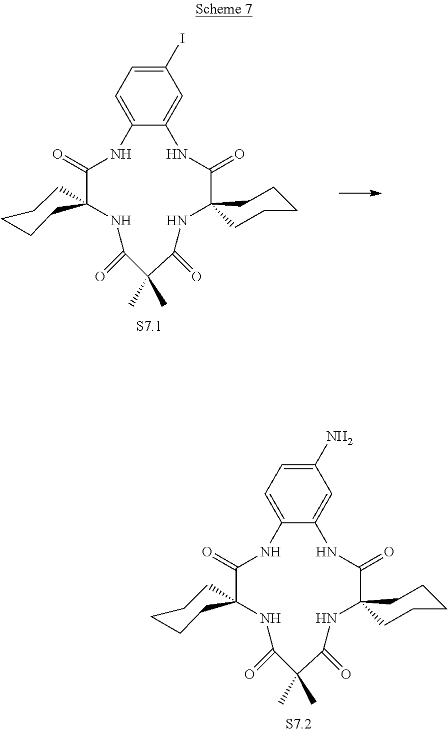

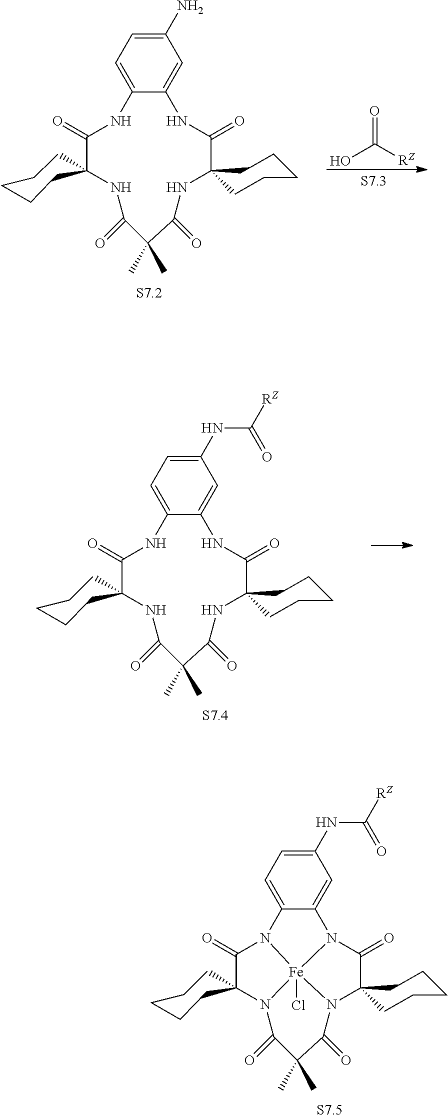

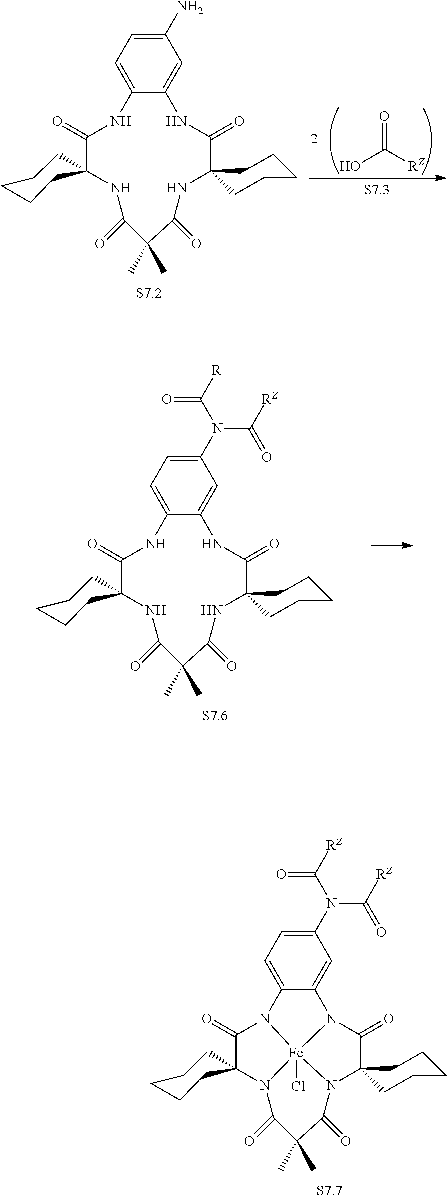

In Scheme 7, compound S7.1 is aminated to compound S7.2 under Buchwald-Hartwig conditions (in the presence of a base and a palladium catalyst). Compound S7.2 is then acylated to the amide S7.4 by the addition of at least 1 equivalent of carboxylic acid S7.3 in the presence of acid. A metal is complexed to compound S7.5 under conditions analogous to those described in U.S. Pat. Nos. 6,100,394 and 8,754,206. For example, S7.4 is dissolved in dry THF, cooled to 0° C., and to this mixture, n-BuLi is added under argon, followed by addition of FeCl₂ to give S7.5.

Compound S7.6 can be made in an analogous manner to compound S7.4 by the addition of at least 2 equivalents of carboxylic acid S7.3 to compound S7.2 in the presence of acid. A metal can then be complexed to compound S7.7 by the same procedure described above for compound S7.5.

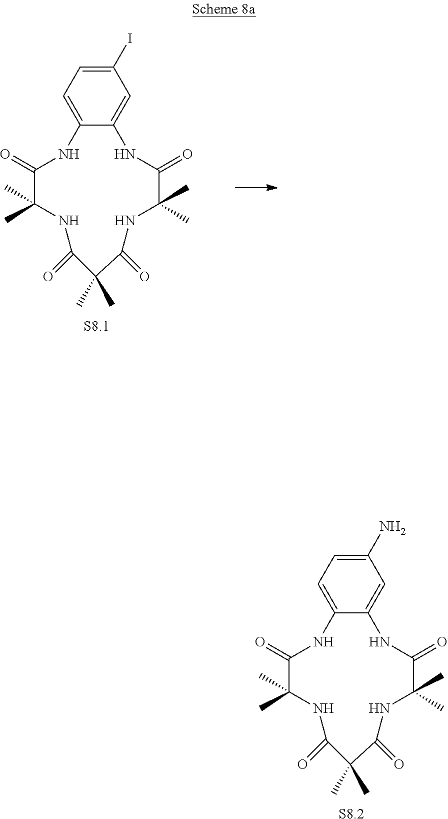

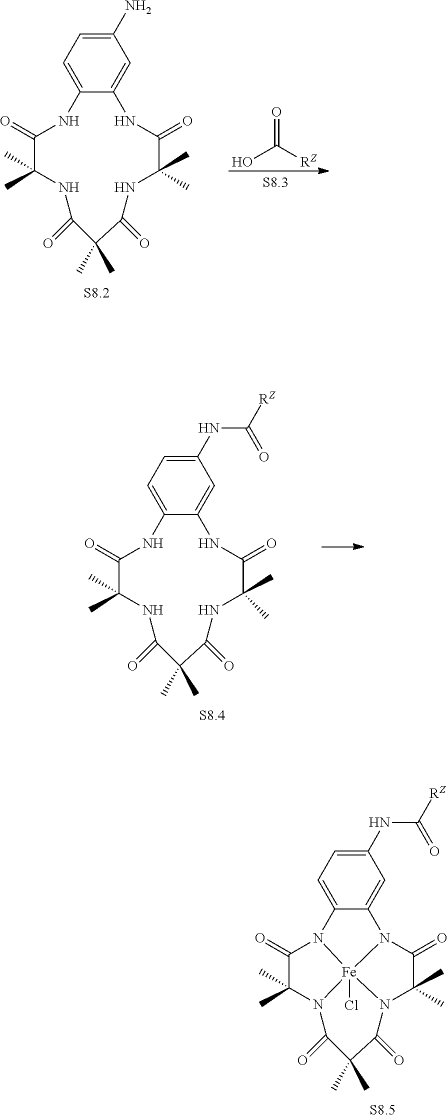

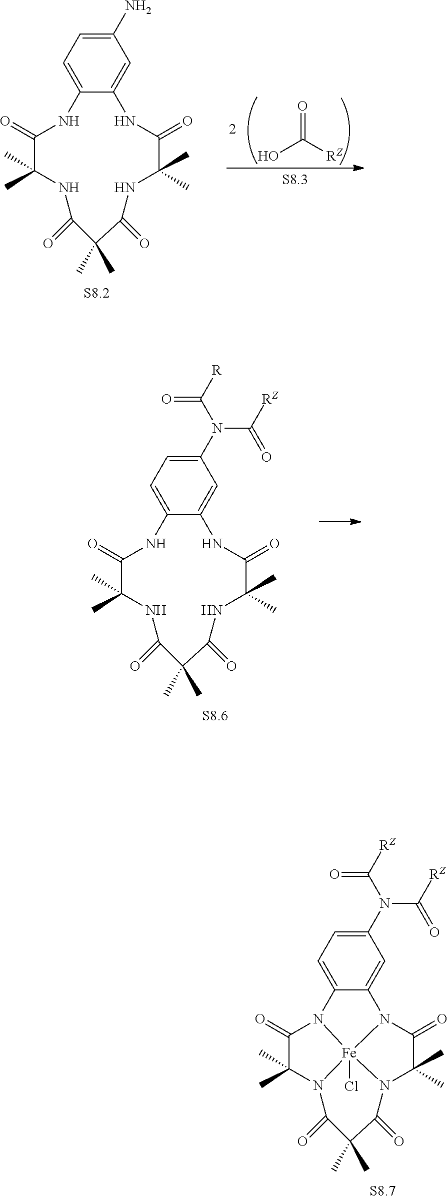

In Scheme 8a, compound S8.1 is aminated to compound S8.2 under Buchwald-Hartwig conditions (in the presence of a base and a palladium catalyst). Compound S8.2 is then acylated to the amide S8.4 by the addition of at least 1 equivalent of carboxylic acid S8.3 in the presence of acid. A metal is complexed to compound S8.5 under conditions analogous to those described in U.S. Pat. Nos. 6,100,394 and 8,754,206. For example, S8.4 is dissolved in dry THF, cooled to 0° C., and to this mixture, n-BuLi is added under argon, followed by addition of FeCl₂ to give S8.5.

Compound S8.6 can be made in an analogous manner to compound S8.4 by the addition of at least 2 equivalents of carboxylic acid S8.3 to compound S8.2 in the presence of acid. A metal can then be complexed to compound S8.7 by the same procedure described above for compound S8.5.

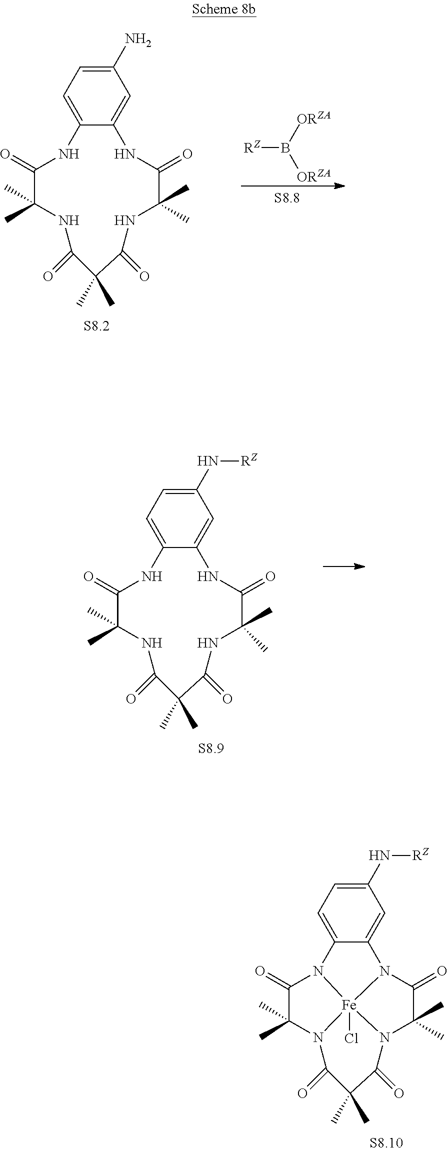

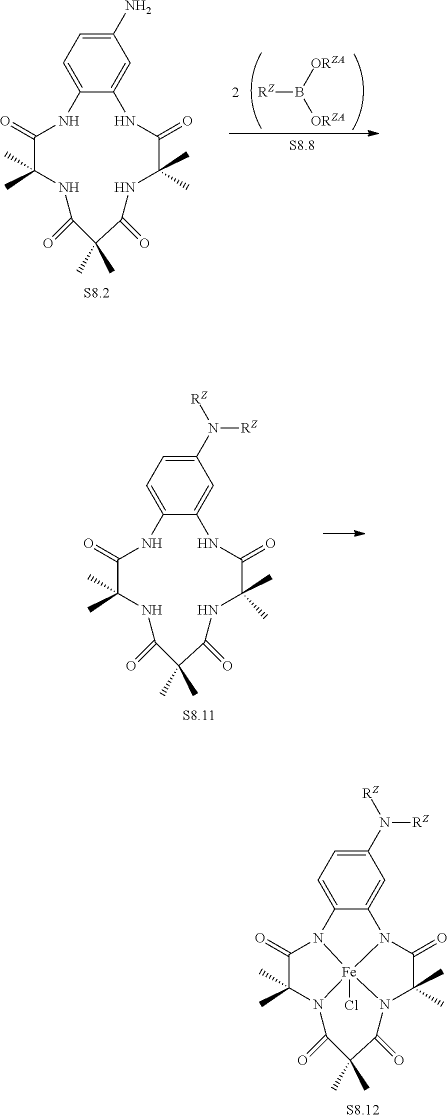

In Scheme 8b, compound S8.2 can undergo a Chan-Lam coupling using at least one equivalent of organoboronate S8.8. R^{ZA} can be hydrogen (thereby forming R^{Z}—B—(OH)₂) or any suitable ligand useful in organoboron cross-coupling reactions. A metal can then be complexed to compound S8.9 by the same procedure described above for compound S8.5 to create compound S8.10. Compound S8.11 can be made in a manner analogous to compound S8.9 by the addition of at least two equivalents of organoboronate S8.8. A metal can then be complexed to compound S8.11 by the same procedure described above for compound S8.5 to create compound S8.12.

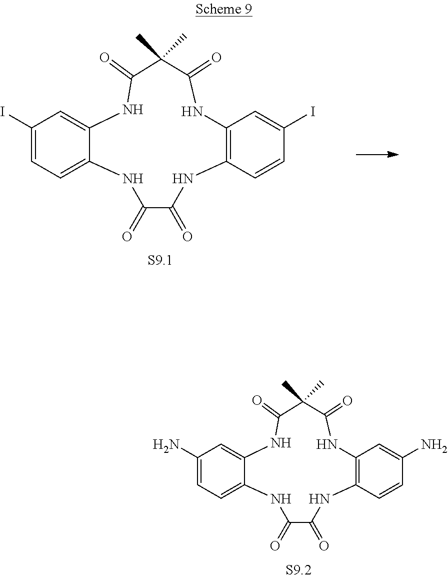

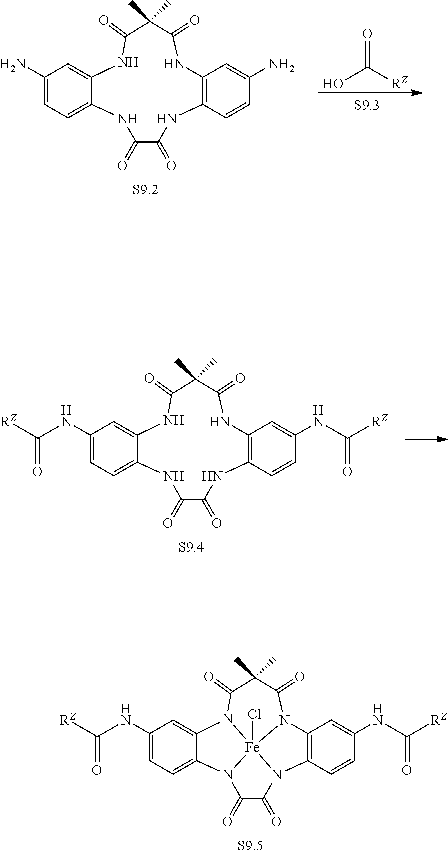

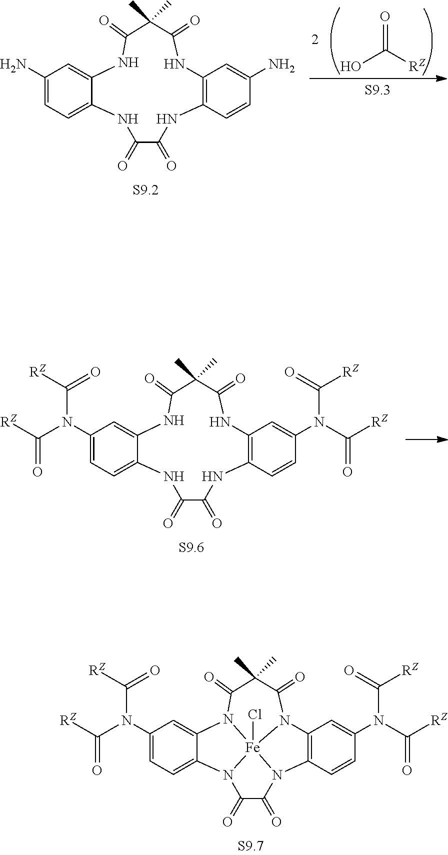

In Scheme 9, compound S8.1 is aminated to compound S8.2 under Buchwald-Hartwig conditions (in the presence of a base and a palladium catalyst). Compound S9.2 is then acylated to the amide S9.4 by the addition of at least 1 equivalent of carboxylic acid S9.3 in the presence of acid. A metal is complexed to compound S9.5 under conditions analogous to those described in U.S. Pat. Nos. 6,100,394 and 8,754,206. For example, S9.4 is dissolved in dry THF, cooled to 0° C., and to this mixture, n-BuLi is added under argon, followed by addition of FeCl₂ to give S9.5.

Compound S9.6 can be made in an analogous manner to compound S9.4 by the addition of at least 2 equivalents of carboxylic acid S9.3 to compound S9.2 in the presence of acid. A metal can then be complexed to compound S9.7 by the same procedure described above for compound S9.5.

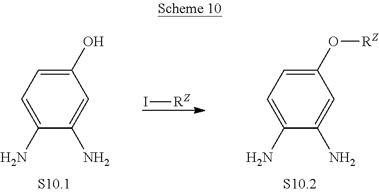

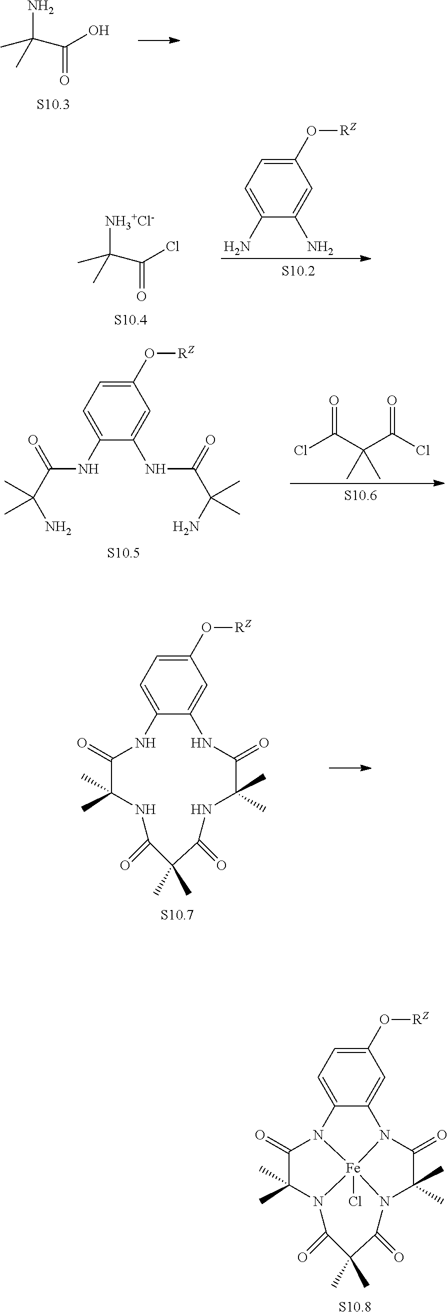

In Scheme 10, compound S10.1 can be alkylated by combining compound S10.1 and S10.2 in the presence of an acid. Compound S10.3 can be acidified with thionyl chloride to form S10.4. The addition of compound S10.4 and compound S10.2 in the presence of a base will form compound S10.5. Exposing compound S10.5 to compound S10.6 in the presence of a base gives compound 10.7. A metal is complexed to compound S10.7 under conditions analogous to those described in U.S. Pat. Nos. 6,100,394 and 8,754,206. For example, S10.7 is dissolved in dry THF, cooled to 0° C., and to this mixture, n-BuLi is added under argon, followed by addition of FeCl₂ to give S10.8.

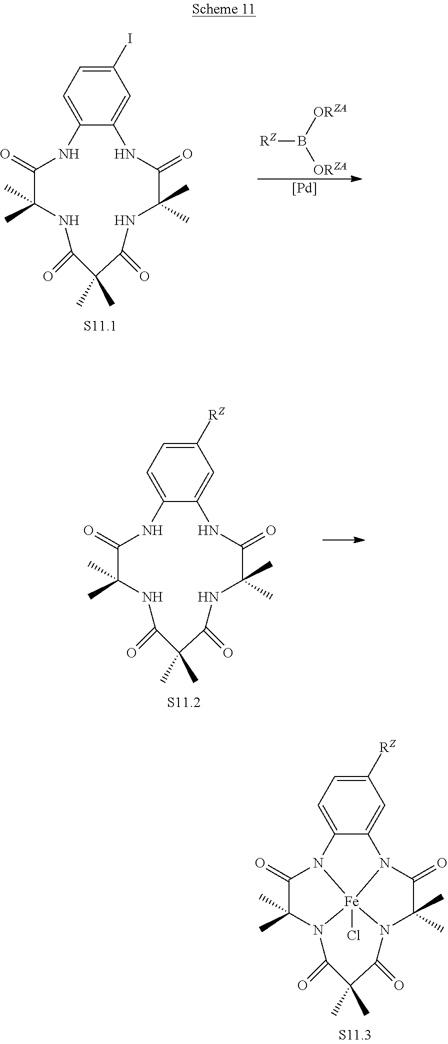

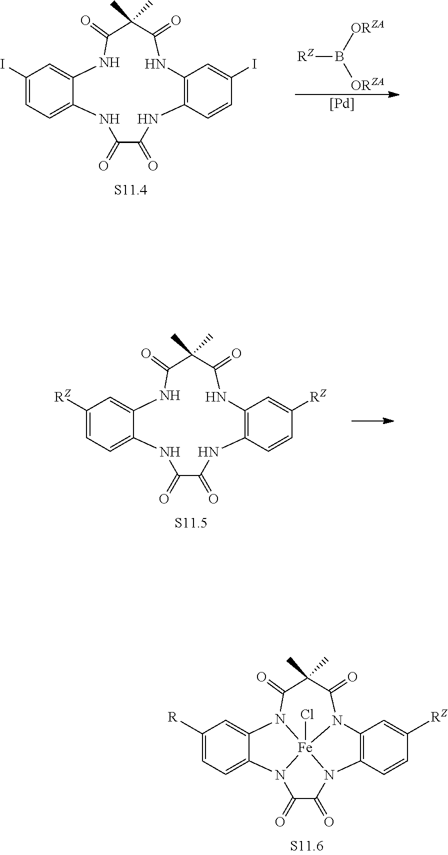

In Scheme 11, compounds S11.1 and S11.4 are converted under Suzuki conditions (exposure of compound S11.1 and S11.4 to the organoboronate R^{Z}—B—(OR^{ZA})₂ in the presence of a palladium catalyst and a base) to compounds S11.2 and S11.5, respectively. R^{ZA} can be hydrogen (thereby forming R^{Z}—B—(OH)₂) or any suitable ligand useful in organoboron cross-coupling reactions. A metal is complexed to compounds S11.2 and S11.5 under conditions analogous to those described in U.S. Pat. Nos. 6,100,394 and 8,754,206 to give compounds S11.3 and S11.6, respectively. For example, S11.2 is dissolved in dry THF, cooled to 0° C., and to this mixture, n-BuLi is added under argon, followed by addition of FeCl₂ to give S11.3.

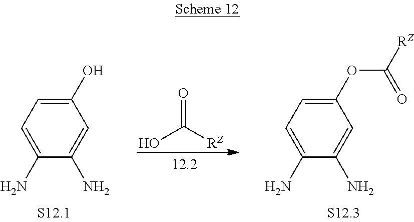

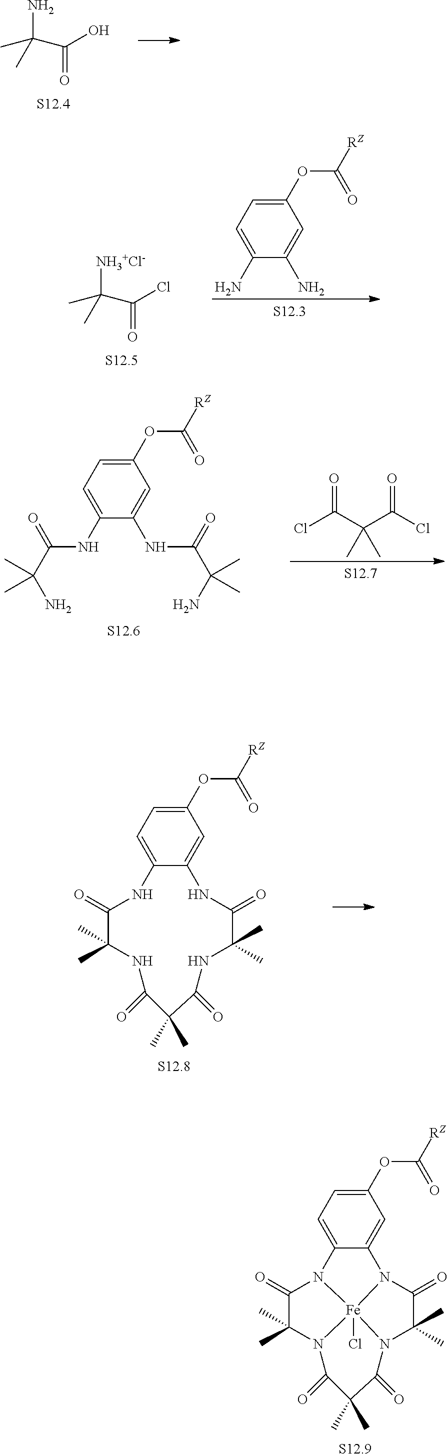

In Scheme 12, compound S12.1 is esterified by combining compound S12.1 and carboxylic acid S12.2 in the presence of an acid to produce compound S12.3. Compound S12.4 can be acidified with thionyl chloride to form S12.5. The addition of compound S12.5 and compound S12.3 in the presence of a base will form compound S12.6. Exposing compound S12.6 to compound S12.7 in the presence of a base gives compound 12.8. A metal is complexed to compound S12.8 under conditions analogous to those described in U.S. Pat. Nos. 6,100,394 and 8,754,206. For example, S10.7 is dissolved in dry THF, cooled to 0° C., and to this mixture, n-BuLi is added under argon, followed by addition of FeCl₂ to give S12.9.

### V. LUMINOPHORES AND ABSORBERS, AND PRECURSORS THEREOF

In some embodiments, the nanoparticles described herein further comprise a luminophore (e.g., a chemiluminophore), which is an atom or functional group in a chemical compound that is responsible for its luminescent properties upon electromagnetic excitation (e.g., light, radiative, or non-radiative intersystem energy transfer such as Forster resonance energy transfer, or thermal excitation), the luminophore (e.g., a dye or a fluorophore) releases a detectable signal. A fluorophore is a fluorescent chemical compound that can emit light upon excitation. Excitation may be optical or chemical in nature.

Examples of the luminophores include fluorescein, rhodamine, resorufin, AlexaFluor, BODIPY, Cy, dansyl, SYTO, chloro-9,10-diphenylanthracene, chloro-9,10-bis(phenylethynyl)anthracene, dichloro-9,10-bis(phenylethynyl)anthracene, rubrene, 5,12-bis(phenylethynyl)naphthacene. Still other exemplary luminophores include: 9,10-diphenylanthracene (DPA); 1-chloro-9,10-diphenylanthracene (1-chloro(DPA)); 2 chloro-9,10-diphenylanthracene (2-chloro(DPA)); 9,10-bis(phenylethynyl)anthracene (BPEA); 1-chloro-9,10-bis(phenylethynyl)anthracene; 2-chloro-9,10-bis(phenylethynyl)anthracene; 1,8-dichloro-9,10-bis(phenylethynyl)anthracene; 2,4-di-tert-butylphenyl 1,4,5,8-tetracarboxynaphthalene diamide; Rhodamine B; 5,12-bis(phenylethynyl)naphthacene; Violanthrone; 16,17-(1,2-ethylenedioxy)violanthrone; 16,17-dihexyloxyviolanthrone; 16,17-butyloxyviolanthrone; N,N′-bis(2,5,-di-tert-butylphenyl)-3,4,9,10-perylenedicarboximide; 1 N,N-dibutylaminoanthracene; 6-methylacridinium iodide; luminol, and coumarin.

Exemplary luminophores include those provided in Table 1, or derivatives thereof, and those disclosed in US 20100171043, which is incorporated by reference herein.

TABLE 1

| Fluorophore | Absorption | Emission | Other info |
|---|---|---|---|
| 1,5 IAEDANS | 336 | 490 | |
| 1,8-ANS | 372 | 480 | |
| 4-Methylumbelliferone | 385 | 502 | |
| 5-carboxy-2,7-dichlorofluorescein | 504 | 529 | |
| 5-Carboxyfluorescein (5-FAM) | 492 | 518 | |
| 5-Carboxynapthofluorescein (pH 10) | 512/598 | 563/668 | Ratio Dye, pH |
| 5-Carboxytetramethylrhodamine (5-TAMRA) | 542 | 568 | |
| 5-FAM (5-Carboxyfluorescein) | 492 | 518 | |
| 5-HAT (Hydroxy Tryptamine) | 370-415 | 520-540 | |
| 5-Hydroxy Tryptamine (HAT) | 370-415 | 520-540 | |
| 5-ROX (carboxy-X-rhodamine) | 578 | 604 | |
| | 567 | 591 | |
| 5-TAMRA (5-Carboxytetramethylrhodamine) | 548 | 552 | |
| | 542 | 568 | |
| 6-Carboxyrhodamine 6G | 518 | 543 | |
| 6-CR 6G | 518 | 543 | |
| 6-JOE | 520 | 548 | |
| 7-amino-4-methylcoumarin | 351 | 430 | |
| 7-Aminoactinomycin D (7-AAD) | 546 | 647 | |
| 7-Hydroxy-4-methylcoumarin | 360 | 449, 455 | |
| 9-Amino-6-chloro-2-methoxyacridine | 412, 430 | 471, 474 | |
| ABQ | 344 | 445 | |
| Acid Fuchsin | 540 | 630 | |
| ACMA (9-Amino-6-chloro-2-methoxyacridine) | 412, 430 | 471, 474 | |
| Acridine Orange + DNA | 502 | 526 | |
| Acridine Orange + RNA | 460 | 650 | |
| Acridine Orange, both DNA & RNA | 440-480 | 520-650 | |
| Acridine Red | 455-600 | 560-680 | |
| Acridine Yellow | 470 | 550 | |
| Acriflavin | 436 | 520 | |
| Acriflavin Feulgen SITSA | 355-425 | 460 | |
| Aequorin (Photoprotein) | | 466 | Photoprotein |
| AFPs—AutoFluorescent Protein - | | | |
| (Quantum Biotechnologies) | | | |
| see sgGFP, sgBFP | | | |
| Alexa Fluor 350 ™ | 346 | 442 | |
| | 342 | 441 | |
| Alexa Fluor 430 ™ | 431 | 540 | |
| Alexa Fluor 488 ™ | 495, 492 | 519, 520 | |
| Alexa Fluor 532 ™ | 531, 532 | 553, 554 | |
| Alexa Fluor 546 ™ | 556, 557 | 572, 573 | |
| Alexa Fluor 568 ™ | 577, 578 | 603 | |
| Alexa Fluor 594 ™ | 590, 594 | 617, 618 | |
| Alexa Fluor 633 ™ | 632 | 650 | |
| Alexa Fluor 647 ™ | 647 | 666 | |
| Alexa Fluor 660 ™ | 668 | 698 | |
| Alexa Fluor 680 ™ | 679 | 702 | |
| Alizarin Complexon | 530-560, 580 | 580, 624-645 | |
| Alizarin Red | 530-560 | 580 | |
| Allophycocyanin (APC) | 630, 645 | 655, 660 | |
| AMC, AMCA-S | 345 | 445 | |
| AMCA (Aminomethylcoumarin) | 345 | 425 | |
| | 347 | 444 | |
| AMCA-X | 353 | 442 | |
| Aminoactinomycin D | 555 | 655 | |
| Aminocoumarin | 346 | 442 | |
| | 350 | 445 | |
| Aminomethylcoumarin (AMCA) | 345 | 425 | |
| | 347 | 444 | |
| Anilin Blue | | 600 | |
| Anthrocyl stearate | 360-381 | 446 | |
| APC (Allophycocyanin) | 630, 645 | 655, 660 | |
| APC-Cy7 | 625-650 | 755 | |
| APTRA-BTC = Ratio Dye, Zn2+ | 466/380 | 520/530 | Ratio Dye, Zn2+ |
| APTS | 424 | 505 | |
| Astrazon Brilliant Red 4G | 500 | 585 | |
| Astrazon Orange R | 470 | 540 | |
| Astrazon Red 6B | 520 | 595 | |
| Astrazon Yellow 7 GLL | 450 | 480 | |
| Atabrine | 436 | 490 | |
| ATTO-TAG ™ CBQCA | 465 | 560 | |
| ATTO-TAG ™ FQ | 486 | 591 | |
| Auramine | 460 | 550 | |
| Aurophosphine G | 450 | 580 | |
| Aurophosphine | 450-490 | 515 | |
| BAO 9 (Bisaminophenyloxadiazole) | 365 | 395 | |
| BCECF (high pH) | 492, 503 | 520,528 | |
| BCECF (low pH) | 482 | 520 | |
| Berberine Sulphate | 430 | 550 | |
| Beta Lactamase | 409 | 447, 520 | |
| BFP blue shifted GFP (Y66H) | 381, 382, 383 | 445, 447, 448 | blue shifted GFP |
| Blue Fluorescent Protein | | | (Y66H) |
| | | | Blue Fluorescent |
| | | | Protein |
| BFP/GFP FRET | | | |
| Bimane | 398 | 490 | |
| Bisbenzamide | 360 | 461 | |
| Bisbenzimide (Hoechst) | 360 | 461 | |
| bis-BTC = Ratio Dye, Zn2+ | 455/405 | 529/505 | Ratio Dye, Zn2+ |
| Blancophor FFG | 390 | 470 | |
| Blancophor SV | 370 | 435 | |
| BOBO ™ -1 | 462 | 481 | |
| BOBO ™ -3 | 570 | 602 | |
| Bodipy 492/515 | 490 | 515 | |
| Bodipy 493/503 | 533 | 549 | |
| Bodipy 500/510 | 509 | 515 | |
| Bodipy 505/515 | 502 | 510 | |
| Bodipy 530/550 | 528 | 547 | |
| Bodipy 542/563 | 543 | 563 | |
| Bodipy 558/568 | 558 | 569 | |
| Bodipy 564/570 | 564 | 570 | |
| Bodipy 576/589 | 579 | 590 | |
| Bodipy 581/591 | 584 | 592 | |
| Bodipy 630/650-X | 625 | 642 | |
| Bodipy 650/665-X | 647 | 665 | |
| Bodipy 665/676 | 605 | 676 | |
| Bodipy FI | 504, 505 | 511, 513 | |
| Bodipy FL ATP | 505 | 514 | |
| Bodipy FI-Ceramide | 505 | 511 | |
| Bodipy R6G SE | 528 | 547 | |
| Bodipy TMR | 542 | 574 | |
| Bodipy TMR-X conjugate | 544 | 573 | |
| Bodipy TMR-X, SE | 544 | 570 | |
| Bodipy TR | 589 | 617 | |
| Bodipy TR ATP | 591 | 620 | |
| Bodipy TR-X SE | 588 | 616 | |
| BO-PRO ™ -1 | 462 | 481 | |
| BO-PRO ™ -3 | 544 | 570 | |
| Brilliant Sulphoflavin FF | 430 | 520 | |
| BTC - Ratio Dye Ca2+ | 464/401 | 533/529 | Ratio Dye Ca2+ |
| BTC-5N - atio Dye, Zn2+ | 459/417 | 517/532 | Ratio Dye, Zn2+ |
| Calcein | 494 | 517 | |
| Calcein Blue | 373 | 440 | |
| Calcium Crimson ™ | 588, 589 | 611, 615 | |
| Calcium Green | 501, 506 | 531 | |
| Calcium Green-1 Ca2+ Dye | 506 | 531 | Ca2+ Dye |
| Calcium Green-2 Ca2+ | 506/503 | 536 | Ca2+ |
| Calcium Green-5N Ca2+ | 506 | 532 | Ca2+ |
| Calcium Green-C18 Ca2+ | 509 | 530 | Ca2+ |
| Calcium Orange | 549 | 575 | |
| | | 576 | |
| Calcofluor White | 385, 395, 405 | 437, 440, 445 | |
| Carboxy-X-rhodamine (5-ROX) | 576 | 601 | |
| Cascade Blue ™ | 377 | 420 | |
| | 398 | 423 | |
| | 399 | | |
| Cascade Yellow | 399 | 550 | |
| | 400 | 552 | |
| Catecholamine | 410 | 470 | |
| CCF2 (GeneBlazer) | | | |
| CFDA | 494 | 520 | |
| CFP—Cyan Fluorescent Protein | 430, 433, 436, | 474, 475, 476, | Cyan Fluorescent |
| | (453) | (501) | Protein |
| CFP/YFP FRET | | | |
| Chlorophyll | 480 | 650 | |
| Chromomycin A | 436-460 | 470 | |
| Chromomycin A | 445 | 575 | |
| CL-NERF (Ratio Dye, pH) | 504/514 | 540 | Ratio Dye, pH |
| CMFDA | 494 | 520 | |
| Coelenterazine Ca2+ Dye, bioluminescence | (429) | 465 | Ca2+ Dye, |
| | | | bioluminescence, |
| | | | native molecule |
| Coelenterazine cp (Ca2+ Dye,) | (430) | 442 | Ca2+ Dye, |
| | | | bioluminescence |
| Coelenterazine f | (437) | 473 | Ca2+ Dye, |
| | | | bioluminescence |
| Coelenterazine fcp | | 452 | Ca2+ Dye, |
| | | | bioluminescence |
| Coelenterazine h | (437) | 464 | Ca2+ Dye, |
| | | | bioluminescence |
| Coelenterazine hcp | (433) | 444 | Ca2+ Dye, |
| | | | bioluminescence |
| Coelenterazine ip | | 441 | Ca2+ Dye, |
| | | | bioluminescence |
| Coelenterazine n | (431) | 467 | Ca2+ Dye, |
| | | | bioluminescence |
| Coelenterazine O | 460 | 575 | |
| Coumarin Phalloidin | 387 | 470 | |
| C-phycocyanine | | | |
| CPM Methylcoumarin | 384 | 469 | Methylcoumarin |
| CTC | 400-450 | 602 | |
| CTC Formazan | | | |
| Cy2 ™ | 489 | 506 | |
| Cy3.1 8 | 554 | 568 | |
| Cy3.5 ™ | 581 | 598 | |
| Cy3 ™ | 514 | 566 | |
| | 552 | 570 | |
| | 554 | | |
| Cy5.1 8 | 649 | 666 | |
| Cy5.5 ™ | 675 | 695 | |
| Cy5 ™ | 649 | 666 | |
| | | 670 | |
| Cy7 ™ | 710, 743 | 767, 805 | |
| Cyan GFP | 433 (453) | 475 (501) | |
| cyclic AMP Fluorosensor (FiCRhR) | 500 | 517 | |
| CyQuant Cell Proliferation Assay | 480 | 520 | Cell Proliferation |
| | | | Assay |
| Dabcyl | 453 | | |
| Dansyl | 340 | 578 | |
| Dansyl Amine | 337 | 517 | |
| Dansyl Cadaverine | 335 | 518 | |
| Dansyl Chloride | 372 | 518 | |
| Dansyl DHPE | 336 | 517 | |
| Dansyl fluoride | 356 | none | |
| DAPI | 359 | 461 | |
| Dapoxyl | 403 | 580 | |
| Dapoxyl 2 | 374 | 574 | |
| Dapoxyl 3 | 373 | 574 | |
| DCFDA | 504 | 529 | |
| DCFH (Dichlorodihydrofluorescein Diacetate) | 505 | 535 | |
| DDAO | 463 | 607 | |
| DHR (Dihydorhodamine 123) | 505 | 534 | |
| Di-4-ANEPPS | 496 | 705 | |
| Di-8-ANEPPS (non-ratio) | 488 | 605 | |
| | 498 | 713 | |
| DiA (4-Di-16-ASP) | 456 | 591 | |
| Dichlorodihydrofluorescein Diacetate (DCFH) | 505 | 535 | |
| DiD - Lipophilic Tracer | 644 | 665 | Lipophilic Tracer |
| DiD (DilC18(5)) | 644 | 665 | |
| DIDS | 341 | 415 | |
| Dihydorhodamine 123 (DHR) | 505 | 535 | |
| Dil (DilC18(3)) | 549, 551 | 565 | |
| Dinitrophenol | 349 | | |
| DiO (DiOC18(3)) | 484, 487 | 501, 502 | |
| DiR | 748 | 780 | Lipophilic Tracer |
| DiR (DilC18(7)) | 750 | 779 | |
| DM-NERF (high pH) | 497/510 | 540 | Ratio Dye, pH |
| DNP | 349 | | |
| Dopamine | 340 | 490-520 | |
| DsRed | 558 | 583 | Red fluorescent |
| DTAF | 494 | 520 | |
| DY-630-NHS | 621 | 660 | Hemicyane |
| | | | label for proteins and |
| | | | DNA |
| DY-635-NHS | 634 | 664 | Hemicyane |
| | | | label for proteins and |
| | | | DNA |
| EBFP | 383 | 447 | Enhanced Blue |
| | | | Fluorescent Protein |
| ECFP | 436 | 474 | Enhanced Cyan |
| | | | Fluorescent Protein |
| EGFP | 488, 498 | 507, 516 | Enhanced Green |
| | | | Fluorescent Protein |
| ELF 97 | 345 | 530 | |
| Eosin | 524 | 545 | |
| Erythrosin | 529, 532 | 554, 555 | |
| Erythrosin ITC | 529 | 555 | |
| Ethidium Bromide | 510, 523 | 595, 605 | |
| Ethidium homodimer -1 (EthD-1) | 528 | 617 | |
| Euchrysin | 430 | 540 | |
| EukoLight | | | |
| Europium (III) chloride | | | |
| EYFP | 513, 520 | 527, 532 | Enhanced Yellow |
| | | | Fluorescent Protein |
| Fast Blue | 360 | 440 | |
| FDA | 494 | 520 | |
| Feulgen (Pararosaniline) | 570 | 625 | |
| FIF (Formaldehyd Induced Fluorescence) | 405 | 433 | |
| FITC | 490, 494 | 520, 525 | |
| FITC Antibody | 493 | 517 | |
| Flazo Orange | 375-530 | 612 | |
| Fluo-3 | 480-506, 506 | 520, 527 | |
| Fluo-4 | 494 | 516 | |
| Fluorescein (FITC) | 490, 494 | 520, 525 | |
| Fluorescein Diacetate | 494 | 520 | |
| Fluoro-Emerald | 495 | 524 | |
| Fluoro-Gold (Hydroxystilbamidine) | 361 | 536 | |
| Fluor-Ruby | 555 | 582 | |
| FluorX | 494 | 520 | |
| FM 1-43 ™ | 479 | 598 | |
| FM 4-46 | 515 | 640 | |
| Fura Red ™ (high pH) | 572 | 657 | |
| Fura Red ™/Fluo-3 | | | |
| Fura-2, high calcium | 335 | 505 | Excitation ratio dye |
| Fura-2, low calcium | 363 | 512 | Excitation ratio dye |
| Fura-2/BCECF | | | |
| Genacryl Brilliant Red B | 520 | 590 | |
| Genacryl Brilliant Yellow 10GF | 430 | 485 | |
| Genacryl Pink 3G | 470 | 583 | |
| Genacryl Yellow 5GF | 430 | 475 | |
| GeneBlazer (CCF2) | | | |
| GFP (S65T) | 498 | 516 | |
| GFP red shifted (rsGFP) | 498 | 516 | |
| GFP wild type, non-UV excitation (wtGFP) | 475 | 509 | |
| GFP wild type, UV excitation (wtGFP) | 395 | 509 | |
| GFPuv | 385 | 508 | |
| Gloxalic Acid | 405 | 460 | |
| Granular Blue | 355 | 425 | |
| Haematoporphyrin | 530-560 | 580 | |
| Hoechst 33258 | 345 | 487 | |
| Hoechst 33342 | 347 | 483 | |
| Hoechst 34580 | 392 | 440 | |
| HPTS | 355 | 465 | |
| Hydroxycoumarin | 325-360 | 386-455 | |
| Hydroxystilbamidine (FluoroGold) | 361 | 536 | |
| Hydroxytryptamine | 400 | 530 | |
| Indo-1, high calcium | 330 | 401 | Emission ratio dye |
| Indo-1, low calcium | 346 | 475 | Emission ratio dye |
| Indodicarbocyanine (DiD) | 644 | 665 | |
| Indotricarbocyanine (DiR) | 748 | 780 | |
| Intrawhite Cf | 360 | 430 | |
| JC-1 | 514 | 529 | |
| JO-JO-1 | 530 | 545 | |
| JO-PRO-1 | 532 | 544 | |
| LaserPro | 795 | 812 | |
| Laurodan | 355 | 460 | |
| LDS 751 (DNA) | 543 | 712 | |
| LDS 751 (RNA) | 590 | 607 | |
| Leucophor PAF | 370 | 430 | |
| Leucophor SF | 380 | 465 | |
| Leucophor WS | 395 | 465 | |
| Lissamine Rhodamine | 572, 577 | 591, 592 | |
| Lissamine Rhodamine B | 577 | 592 | |
| LIVE/DEAD Kit Animal Cells | 494 | 517 | for more details refer |
| Calcein/Ethidium homodimer | 528 | 617 | to www.probes.com |
| LOLO-1 | 566 | 580 | |
| LO-PRO-1 | 568 | 581 | |
| Lucifer Yellow | 425, 428 | 528, 536, 540 | |
| Lyso Tracker Blue | 373 | 422 | |
| Lyso Tracker Blue-White | 466 | 536 | |
| Lyso Tracker Green | 504, 534 | 511, 551 | |
| Lyso Tracker Red | 490 | 516 | |
| Lyso Tracker Yellow | 551 | 576 | |
| LysoSensor Blue | 374 | 424 | |
| LysoSensor Green | 442 | 505 | |
| LysoSensor Yellow/Blue | 384 | 540 | |
| Mag Green | 507 | 531 | |
| Magdala Red (Phloxin B) | 524 | 600 | |
| Mag-Fura Red | 483/427 | 659/631 | Ratio Dye, Mg2+ |
| Mag-Fura-2 | 369/329 | 508 | Ratio Dye Ca2+ |
| | 369/330 | 511/491 | Ratio Dye Mg2+ |
| Mag-Fura-5 | 369/330 | 505/500 | Ratio Dye, Ca2+ |
| | 369/332 | 505/482 | Ratio Dye, Mg2+ |
| Mag-Indo-1 | 349/328 | 480/390 | Ratio Dye, Ca2+ |
| | 349/330 | 480/417 | Ratio Dye, Mg2+ |
| Magnesium Green | 506, 507 | 531 | |
| Magnesium Orange | 550 | 575 | |
| Malachite Green | 628 | | |
| Marina Blue | 362 | 459 | |
| Maxilon Brilliant Flavin 10 GFF | 450 | 495 | |
| Maxilon Brilliant Flavin 8 GFF | 460 | 495 | |
| Merocyanin | 555 | 578 | |
| Methoxycoumarin | 360 | 410 | |
| Mitotracker Green FM | 490 | 516 | |
| Mitotracker Orange | 551 | 576 | |
| Mitotracker Red | 578 | 599 | |
| Mitramycin | 450 | 470 | |
| Monobromobimane | 398 | 490 | |
| Monobromobimane (mBBr-GSH) | 398 | 500 | |
| Monochlorobimane | 380 | 461 | |
| MPS (Methyl Green Pyronine Stilbene) | 364 | 395 | |
| NBD | 466 | 539 | |
| NBD Amine | 450 | 530 | |
| Nile Red | 515-555, 559 | 590, 640 | |
| Nitrobenzoxadidole | 465 | 510-650 | |
| Noradrenaline | 340 | 490-520 | |
| Nuclear Fast Red | 289-530 | 580 | |
| Nuclear Yellow | 365 | 495 | |
| Nylosan Brilliant lavin E8G | 460 | 510 | |
| Oregon Green | 503 | 522 | |
| Oregon Green 488-X | 494 | 517 | |
| Oregon Green ™ | 503 | 522 | |
| Oregon Green ™ 488 | 490, 493 | 514, 520 | |
| Oregon Green ™ 500 | 497 | 517 | |
| Oregon Green ™ 514 | 506 | 526 | |
| Pacific Blue | 405 | 455 | |
| Pararosaniline (Feulgen) | 570 | 625 | |
| PBFI | 340/380 | 420 | Excitation ratio dye |
| PE-Cy5 | 488 | 670 | |
| PE-Cy7 | 488 | 755, 767 | |
| PerCP | 488 | 675 | |
| PerCP-Cy5.5 | 488 | 710 | |
| PE-TexasRed [Red 613] | 488 | 613 | |
| Phloxin B (Magdala Red) | 524 | 600 | |
| Phorwite AR | 360 | 430 | |
| Phorwite BKL | 370 | 430 | |
| Phorwite Rev | 380 | 430 | |
| Phorwite RPA | 375 | 430 | |
| Phosphine 3R | 465 | 565 | |
| PhotoResist | 365 | 610 | |
| Phycoerythrin B [PE] | 546-565 | 575 | |
| Phycoerythrin R [PE] | 565 | 578 | |
| PKH26 (Sigma) | 551 | 567 | |
| PKH67 | 496 | 520 | Chroma |
| PMIA | 341 | 376 | |
| Pontochrome Blue Black | 535-553 | 605 | |
| POPO-1 | 433 | 457 | |
| POPO-3 | 533 | 574 | |
| PO-PRO-1 | 435 | 455 | |
| PO-PRO-3 | 539 | 567 | |
| Primuline | 410 | 550 | |
| Procion Yellow | 470 | 600 | |
| Propidium Iodid (PI) | (305), 536, 538 | 617 | |
| PyMPO | 412, 415 | 561, 564, 570 | |
| Pyrene | 360 | 387 | |
| Pyronine | 410 | 540 | |
| Pyronine B | 540-590 | 560-650 | |
| Pyrozal Brilliant Flavin 7GF | 365 | 495 | |
| QSY 7 | 560 | | |
| Quinacrine Mustard | 440 | 510 | |
| Red 613 [PE-TexasRed] | 488 | 613 | |
| Resorufin | 571 | 584, 585 | |
| RH 414 | 532 | 716 | |
| Rhod-2 | 552 | 576 | |
| Rhodamine | 550 | 573 | |
| Rhodamine 110 | 496, 497 | 520 | |
| Rhodamine 123 | 507 | 529 | |
| Rhodamine 5 GLD | 470 | 565 | |
| Rhodamine 6G | 525 | 555 | |
| Rhodamine B | 540 | 625 | |
| Rhodamine B 200 | 523-557 | 595 | |
| Rhodamine B extra | 550 | 605 | |
| Rhodamine BB | 540 | 580 | |
| Rhodamine BG | 540 | 572 | |
| Rhodamine Green | 502 | 527 | |
| Rhodamine Phallicidine | 558, 542 | 575, 565 | |
| Rhodamine Phalloidine | 542 | 565 | |
| Rhodamine Red | 570 | 590 | |
| Rhodamine WT | 530 | 555 | |
| Rose Bengal | 525, 540 | 550-600 | |
| R-phycocyanine | | | |
| R-phycoerythrin (PE) | 565 | 578 | |
| rsGFP | 473 | 509 | red shifted GFP |
| | | | (S65T) |
| S65A | 471 | 504 | |
| S65C | 479 | 507 | |
| S65L | 484 | 510 | |
| S65T | 488 | 511 | |
| Sapphire GFP | 395 | 511 | |
| SBFI | 340/380 | 420 | Excitation ratio dye |
| Serotonin | 365 | 520-540 | |
| Sevron Brilliant Red 2B | 520 | 595 | |
| Sevron Brilliant Red 4G | 500 | 583 | |
| Sevron Brilliant Red B | 530 | 590 | |
| Sevron Orange | 440 | 530 | |
| Sevron Yellow L | 430 | 490 | |
| sgBFP ™ | 387 | 450 | |
| sgBFP ™ (super glow BFP) | 387 | 450 | Quantum's |
| | | | SuperGlo ™GFP |
| | | | AFPs |
| sgGFP ™ | 474 | 488 | |
| sgGFP ™ (super glow GFP) | 474 | 509 | Quantum's |
| | | | SuperGlo ™GFP |
| | | | AFPs |
| SITS | 336 | 436 | Ion Channels |
| SITS (Primuline) | 395-425 | 450 | |
| SITS (Stilbene Isothiosulphonic Acid) | 365 | 460 | |
| SNAFL calcein | 506/535 | 535/620 | Ratio Dye, pH |
| SNAFL-1 | 508/540 | 543/623 | Ratio Dye, pH |
| SNAFL-2 | 514/543 | 546/630 | Ratio Dye, pH |
| SNARF calcein | 552/574 | 590/629 | Ratio Dye, pH |
| SNARF1 | 576/548 | 635/587 | Excitation and |
| | | | emission ratio dye |
| Sodium Green | 506, 507 | 532 | Na+, K+ |
| SpectrumAqua | 433,/53 | 480/55 | Vysis |
| SpectrumGreen | 497/30, 509/31 | 538/44, 524/56 | Vysis |
| SpectrumOrange | 559/38, 560 | 588/48 | Vysis |
| Spectrum Red | 587, 587/35 | 612, 612/51 | |
| SPQ (6-methoxy-N-(3-sulfopropyl) quinolinium) | 344 | 443 | |
| Stilbene | 335 | 440 | |
| Sulphorhodamine B can C | 520 | 595 | |
| Sulphorhodamine G Extra | 470 | 570 | |
| SYTO 11 | 508, 510 | 527, 530 | Dye for DNA, RNA |
| SYTO 12 | 499, 500 | 522, 519 | Dye for DNA, RNA |
| SYTO 13 | 488, 491 | 509, 514 | Dye for DNA, RNA |
| SYTO 14 | 517, 521 | 549, 547 | Dye for DNA, RNA |
| SYTO 15 | 516, 518 | 546, 555 | Dye for DNA, RNA |
| SYTO 16 | 488, 494 | 518, 525 | Dye for DNA, RNA |
| SYTO 17 | 621 | 634 | Dye for DNA |
| SYTO 18 | 490, 493 | 507, 527 | Dye for DNA, RNA |
| SYTO 20 | 512 | 530 | Dye for DNA |
| SYTO 21 | 494 | 517 | Dye for DNA |
| SYTO 22 | 515 | 535 | Dye for DNA |
| SYTO 23 | 499 | 520 | Dye for DNA |
| SYTO 24 | 490 | 515 | Dye for DNA |
| SYTO 25 | 521 | 556 | Dye for DNA |
| SYTO 40 | 420 | 441 | Dye for DNA |
| SYTO 41 | 430 | 454 | Dye for DNA |
| SYTO 42 | 433 | 460 | Dye for DNA |
| SYTO 43 | 436 | 467 | Dye for DNA |
| SYTO 44 | 446 | 471 | Dye for DNA |
| SYTO 45 | 452 | 484 | Dye for DNA |
| SYTO 59 | 622 | 645 | Dye for DNA |
| SYTO 60 | 652 | 678 | Dye for DNA |
| SYTO 61 | 628 | 645 | Dye for DNA |
| SYTO 62 | 652 | 676 | Dye for DNA |
| SYTO 63 | 657 | 673 | Dye for DNA |
| SYTO 64 | 599 | 619 | Dye for DNA |
| SYTO 80 | 531 | 545 | Nucleic Acid Stain |
| SYTO 81 | 530 | 544 | Nucleic Acid Stain |
| SYTO 82 | 541 | 560 | Nucleic Acid Stain |
| SYTO 83 | 543 | 559 | Nucleic Acid Stain |
| SYTO 84 | 567 | 582 | Nucleic Acid Stain |
| SYTO 85 | 567 | 583 | Nucleic Acid Stain |
| SYTOX Blue | 445 | 470 | Nucleic Acid Stain |
| SYTOX Green | 504 | 523 | Nucleic Acid Stain |
| SYTOX Orange | 547 | 570 | Nucleic Acid Stain |
| Tetracycline | 390-425 | 525-560 | |
| Tetramethylrhodamine (TRITC) | 555 | 576 | |
| Texas Red ™ | 595 | 620 | |
| Texas Red-X ™ conjugate | 595 | 615 | |
| Thiadicarbocyanine (DiSC3) | 651, 653 | 674, 675 | |
| Thiazine Red R | 596 | 615 | |
| Thiazole Orange | 510 | 530 | |
| Thioflavin 5 | 430 | 550 | |
| Thioflavin S | 430 | 550 | |
| Thioflavin TCN | 350 | 460 | |
| Thiolyte | 370-385 | 477-488 | |
| Thiozole Orange | 453 | 480 | |
| Tinopol CBS (Calcofluor White) | 390 | 430 | |
| TMR | 550 | 573 | |
| TO-PRO-1 | 515 | 531 | |
| TO-PRO-3 | 644 | 657 | |
| TO-PRO-5 | 747 | 770 | |
| TOTO-1 | 514 | 531, 533 | |
| TOTO-3 | 642 | 660 | |
| Tricolor (PE-Cy5) | (488) 650 | 667 | |
| TRITC (TetramethylRodamineIsoThioCyanate) | 550 | 573 | |
| True Blue | 365 | 425 | |
| TruRed | 490 | 695 | |
| Ultralite | 656 | 678 | |
| Uranine B | 420 | 520 | |
| Uvitex SFC | 365 | 435 | |
| wt GFP | 395 (475) | 508 | wild type GFP |
| WW 781 | 605 | 639 | |
| X-Rhodamine | 580 | 605 | |
| XRITC | 582 | 601 | |
| Xylene Orange | 546 | 580 | |
| Y66F | 360 | 508 | |
| Y66H | 360 | 442 | |
| Y66W | 436 | 485 | |
| Yellow GFP | 513 | 527 | Yellow shifted |
| | | | Green Fluorescent |
| | | | Protein |
| YFP | 513, 520 | 527, 532 | Yellow Fluorescent |
| | | | Protein |
| YO-PRO-1 | 491 | 506 | |
| YO-PRO-3 | 613 | 629 | |
| YOYO-1 | 491 | 508, 509 | |
| YOYO-3 | 612 | 631 | |

In some examples, the luminophore is a luminescent platinum group metal complex with one or more α-diimine ligands, for example, Ruthenium (II) diamine complexes (e.g., ruthenium(II) tris(2,2′-bipyridyl); ruthenium(II) tris(1,10-phenanthroline), and ruthenium(II) tris(4,7-diphenyl-1,10-phenantroline). Alternatively, the luminophore can be a platinum (II) porphyrin, such as platinum(II) octaethylporphyrin or platinum(II) tetrakis(pentafluorophenyl)porphyrin; a palladium(II) porphyrin such as palladium(II) octaethylporphyrin; a cyclometalated iridium (III) coumarin complex, a luminescent lanthanide complex such as europium (III) complex or terbium (III) complex, or a quantum dot.

When a luminophore such as a fluorphore is used, absorptive species generative therefrom can be monitored by irradiation with light of the proper wavelength or by a radiative transfer of energy such as FRET, which would allow for a chemiluminescent species to excite a fluorophore such that no input light would be needed.

In some embodiments, the signal inducing agent used in the assay methods described herein is a luminophore precursor (e.g., a chemiluminophore precursor), which is a molecule that converts to a compound which releases a detectable signal via a physical or chemical reaction. A luminophore precursor may be a precursor species that reacts to yield fluorescent or absorbent species upon release (e.g., at suitable pH value). Examples include, but are not limited to, acylated fluorescein derivatives, acylated SNARF derivatives, or acylated BCECF derivatives, or other compounds as described herein. When placed in a solution having a suitable pH value (containing a suitable acid or base pH modulator), the precursor could convert to a fluorescent or absorbent dye, which is capable of releasing a detectable signal. An energy source such as thermal may be needed to increase the rate of the conversion, which lead to a signal change (fluorescence increase or decrease or absorbance increase or decrease). In other examples, the dye precursor can be a precursor that yields a fluorescent molecule by stoichiometric reaction with an oxidizing agent, reducing agent, and/or a metal. Conversion of the precursor molecule to the fluorescent or absorbent molecule can be performed in a suitable solution (e.g., aqueous based), which may comprise an oxidant, a reductant, or a metal. Examples include hydrogen peroxide, hypochlorous acid, sodium hypochlorite, hydrogen sulfide, dithianes, thiols, glutathione, acetylcysteine, Hg(II), Cu(II), Cu(I), or Co(II).

Still other luminophores, or precursors thereof, include luminol (C₈H₇N₃O₂) and its derivatives, bis(2,4,5-trichlorophenyl-6-carbopentoxyphenyl)oxalate and its derivatives, acridinium and its derivatives, dioxetane and its derivatives, substituted aryl oxalates, coelenterazine and its derivatives, peroxyoxalic derivatives, and Ruthenium(II) complexes such as tris(2,2′-bipyridine).

In some embodiments, the luminophore precursor is a fluorescein or rhodamine compound (e.g., a fluorescein or a rhodamine compound comprising acyl or sulfonyl functional groups that modulate the fluorescence of the compound). In certain embodiments the luminophore precursor is an acylated or alkylated fluorescein or an acylated or alkylated rhodamine.

In other embodiments, the luminophore precursor is a fluorescein compound having a structure according to formula A,

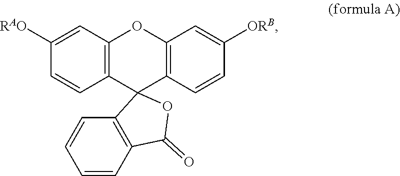

wherein each of R^{A} and R^{B} is, independently, acetyl, propionyl, butyryl, valeryl, hexanoyl, heptanoyl, decanoyl, dodecanoyl, hexadecanoyl, acrylyl, methanesulfonyl, isobutoxy carbonyl, furoyl, benzoyl, or —CH₂OC(═O)CH₃. In some embodiments, each of R^{A} and R^{B} is, independently, —C(O)(CH₂)ₓCH₃, where x is an integer between 0-20. In some embodiments, R^{A} and R^{B} are the same. In other embodiments, R^{A} and R^{B} are different. In still other embodiments, the compound of formula A further comprises 1, 2, or 3 substituent groups selected from halogen (e.g., F, Cl, Br, or I), C₁₋₆ alkyl, and C₁₋₆ alkoxy.

In other embodiments, the fluorescein compound is represented by the following structural formula:

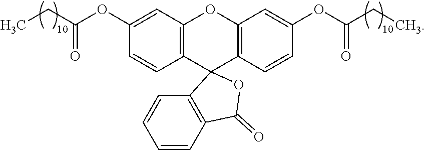

In other embodiments, the luminophore precursor is a fluorescein compound having a structure according to formula B,

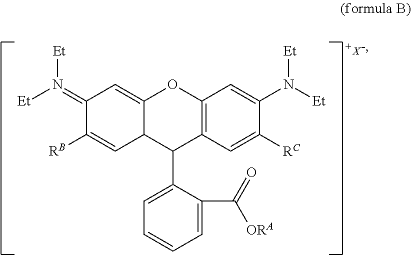

wherein R^{A} is a C₁₋₂₀ alkyl or a 5-10-membered heterocyclyl (e.g., an N-hydroxysuccinimide), and R^{B} and R^{C} are, independently, selected from hydrogen, halogen (e.g., F, Cl, Br, or I), C₁₋₆ alkyl, and C₁₋₆ alkoxy. In some embodiments, R^{B} and R^{C} are the same. In other embodiments, R^{B} and R^{C} are different.

In still other embodiments, the luminophore precursor is fluorescein dilaurate, rhodamine B octadecyl ester, or rhodamine B hexyl ester.

In other embodiments, the luminophore precursor is a coumarin compound having a structure according to formula C:

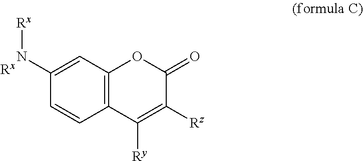

wherein each R^{x} is independently hydrogen or C₁-C₃ alkyl, R^{y} is hydrogen or —CF₃, and R^{z} is hydrogen or a 5-10 membered heterocyclyl. In some embodiments, both R^{X} are the same. In other embodiments, R^{z} is benzothiazolyl or benzimidazolyl optionally substituted with methyl. In still other embodiments, R^{z} is

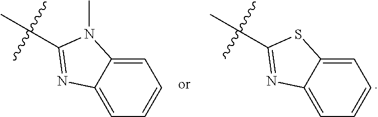 [SMILES: *c1nc2ccccc2n1C ; *c1nc2ccccc2s1]

In other embodiments, the luminophore precursor is selected from the following:

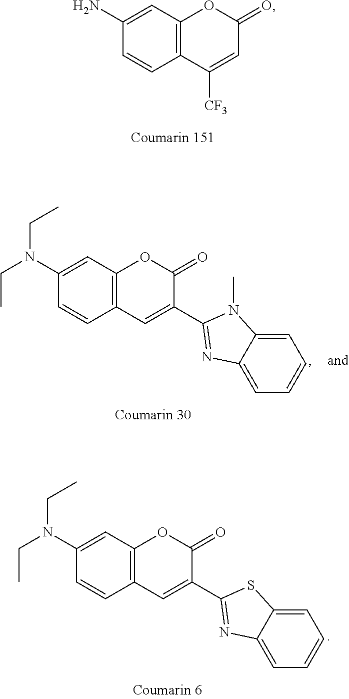 [SMILES: Nc1ccc2c(C(F)(F)F)cc(=O)oc2c1 ; CCN(CC)c1ccc2cc(-c3nc4ccccc4n3C)c(=O)oc2c1 ; CCN(CC)c1ccc2cc(-c3nc4ccccc4s3)c(=O)oc2c1]

In still other embodiments, the luminophore or luminophore precursor is a compound selected from: Oregon green, eosin, Texas red, BODIPY, AlexaFluor, Atto, cyanine, indocarbocyanine, oxacarbocyanine, thiacarbocyanine, merocyanine, dansyl, prodan, coumarin, pyridyloxazole, nitrobenzoxadiazole, benzoxadiazole, anthraquinone, cascade blue, Nile red, Nile blue, cresyl violet, proflavin, acridine orange, acridine yellow, auramine, crystal violet, malachite green, porphin, phthalocyanine, bilirubin, 9,10-diphenylanthracene, 1-chloro-9,10-diphenylanthracene, 9,10-bis(phenylethynyl)anthracene, 1-chloro-9,10-bis(phenylethynyl)anthracene, 2-chloro-9,10-bis(phenylethynyl)anthracene, 1,8-dichloro-9,10-bis(phenylethynyl)anthracene, rubrene, 2,4-di-tert-butylphenyl-1,4,5,8-tetracarboxynaphthalene diamie, 5,12-bis(phenylethynyl)naphthacene, violanthrone, 16,16-(1,2-ethylenedioxy)violanthrone, 16,17-dihexyloxyviolanthrone, 16,17-butyloxyviolanthrone, N,N′-bis(2,5-di-tert-butylphenyl)-3,4,9,10-perylenedicarboximide, 1-N,N′-dibutylaminoanthracene, 6-methylacridinium iodide, and luminol, or a derivative thereof (e.g., an acylated, alkylated, alkoxylated, and/or halogenated derivative of the compounds described herein).

### VI. ASSAY METHODS

The nanoparticles and liposomes described herein may be used with multiple chemical and/or biochemical assay formats and/or platforms including, but not limited to, well-, microwell-, microfluidic-, gel-, magnetic particle-, solid chromatographic-based assay formats, for detecting and quantifying analytes of interest in a sample. Assay types may include, but are not limited to, sandwich, hybridization, competition, and other assays.

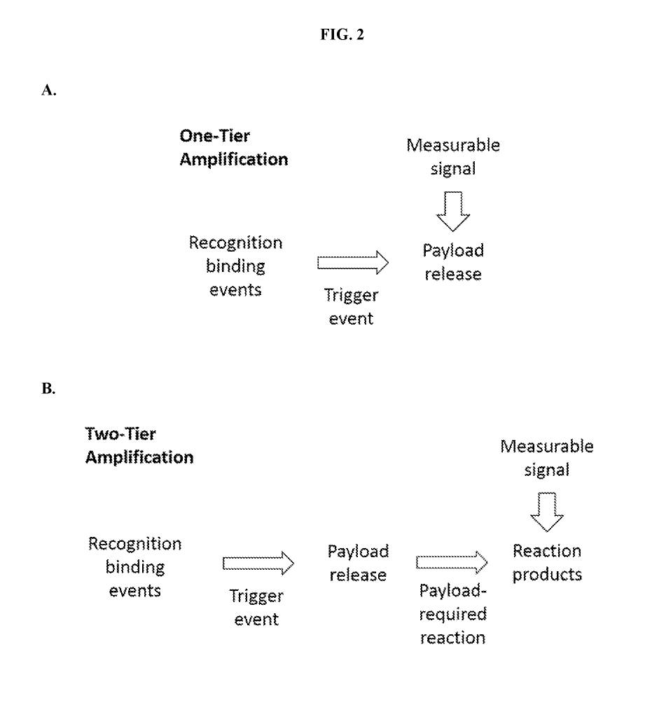

Depending upon the type of the signal inducing agent used in the nanoparticles or liposomes, the away method can be a one-tier amplification assay or a two-tier amplification assay. See examples in FIG. 2A-B. The ratio of the number of payload species to binding events dictates the amplification of the signal, termed a “one-tier” amplification (401) (FIG. 2A).

Examples include the release of specific ions that can be electrically or optically detected including, but not limited to, F, Cu⁺, Cu²⁺, Fe²⁺, Fe³⁺, NO₃⁻, SO₄²⁺, NH₄⁺, Hg²⁺, Ti²⁺, Ti⁴⁺, S⁻, Ca²⁺, H⁺, Au²⁺, Ag⁺, Pd²⁺, Pt²⁺, etc. In order to enhance optical detection, ions may complex with species in the solution, such as the aqueous cupric ammonium ion.

The signal inducing agent may also participate in one or more reactions that produce one or more measurable signals. The signals may be optical, electrical, magnetic, acoustic, or other. The payloads may be reagents or catalysts in the reaction(s) that produce the signals, with catalysis the preferred mode of operation. They may be molecular, ionic, or particulate in nature. The signal inducing agent may result in a reaction that either increases or decreases the measured signal. Examples of reactions include, but are not limited to, oxidation, reduction, addition, elimination, polymerization, and/or rearrangement chemistries. The signal amplification may thus be two-fold or “two-tier” (501): the first level is based on the ratio of the number of payload species to binding events and the second level is based on the reaction(s) in which the payload species participate. FIG. 2B. The addition of a “stop chemistry” may be required to terminate the reaction for optimal detection.

Nanoparticles or liposomes with signal inducing agents that produce two-tier amplification may require reagents to be added to the sample being tested. These reagents may be added before, during, or after the biochemical binding event(s). In order to control the timing of the onset of the reaction, one or more reagents may be contained in an inactive state, such as protected in a particle or polymer, until the onset of a defined trigger. Suitable triggers are the same as those that release signal inducing agents. Such “reagent vessels” may contain surface molecules that participate in the biochemical binding event(s). They may also contain magnetic particles to enable magnetically-driven assay control.

Multiple assays may be run in parallel and/or serially. Control assays may validate assay performance and/or provide and/or enhance quantification. Species other than the “detection species,” termed “tracers,” may be present for these controls.

Assay and/or particle design may also enable multiplexed detection to be performed. Labels may respond to similar or different triggers, may containing similar or different payloads, and/or may contain similar or different tracers. For bead-based assays, tracers may be present on beads that participate in the assays. Tracers may be used to tune the number of labels available.

Microfluidic assays may be performed on a cartridge designed to spin. Such centrifugal forces may be used to drive fluid flow and/or contain reactions. The spin speed may be used to control the assays, isolating reactions and determining reaction times. Such fluid control may be defined by elements like, but not limited to, flow time through microfluidic paths, soluble plugs with defined dissolution times, plugs that open with sufficient pressure, etc.

(i) Sandwich Assays

In some embodiments, the assay methods described herein are carried out in a sandwich format, which is suitable for detecting a relatively large analyte, which allows for binding to two binding agents, such as two antibodies that bind to different epitopes of the analyte. Performing a sandwich assay typically involves at least one binding agent (e.g., an antibody) with specificity for an analyte of interest for detection (the detection agent). The sample with an unknown amount of the analyte can be immobilized on a solid support (e.g., a polystyrene microtiter plate) either non-specifically (via adsorption to the surface) or specifically (via a capture agent that specifically binds the analyte, such as an antibody). After the analyte is immobilized, the detection agent is added, forming a complex with the analyte. The detection agent can be covalently linked to the nanoparticle as described herein. Between each step, the solid support such as the plate is typically washed with a mild detergent solution to remove any agent such as antibodies that are non-specifically bound. After the final wash step, the nanoparticle can be dissociated as described herein to release the signal inducing agent, which can be subjected to a reaction resulting in a signal change. The signal change indicates the presence and/or quantity of the analyte in the sample.

To perform the Sandwich assay as described herein, a sample suspected of containing an analyte of interest can be incubated with a solid support, e.g., a microwell plate, on which a capture agent (e.g., an antibody specific to the analyte) is immobilized. The solid support can then be washed to remove free analytes. The nanoparticle, which is conjugated to a binding agent specific to the analyte, can be incubated with the solid support under suitable conditions allowing for the binding of the binding agent to the analyte of interest captured on the solid support. In some examples, both the capture agent and the binding agent attached to the nanoparticles or liposomes are antibodies which bind to different epitopes of the analyte. After removal of any free nanoparticles or liposomes from the mixture, the nanoparticles or liposomes captured on the solid support (via binding to any analyte of interest captured on the solid support) are dissociated by a suitable trigger (e.g., a chemical trigger, a physical trigger, or a combination thereof) to release the signal inducing agent entrapped in the nanoparticles or liposomes. Selection of a suitable trigger would depend on the type of nanoparticles or liposomes used in the assay, which is within the knowledge of those skilled in the art. For example, if the nanoparticle or liposomes contains a hollow core having air or liquid, ultrasound can be used to dissociate such nanoparticles or liposomes. In another example, if the nanoparticle or liposomes contains a dopant, a physical trigger (e.g., light or thermal) and/or a chemical trigger (e.g., solvent or pH) can be used to dissociate the nanoparticles. Provided below is a table showing exemplary release designs and conditions that may be needed for dissociating the nanoparticles or liposomes (Table 2):

TABLE 2

| | | Solution components required for |
|---|---|---|
| Release design | Energy required for release | release |
| Thermal | Heat - may be conductive, | Any solvent may be used |
| | radiative, convective | |
| Chemical | Not required, though heat may | Presence of solvent, which may be pH- |
| | be used to enhance degradation | tuned, directly degrades NP, releasing |
| | reaction | cargo |
| Thermal + chemical | Heat - may be conductive, | Specific solubilizing solvent for NP |
| | radiative, convective | must be present - for example organic |
| | | component such as IPA, EtOH, DMSO |
| | | must be added |
| Light + chemical | Light - likely in UV region | Solvent required to solubilize degraded |
| | degrades NP backbone or | NP components, which may be pH- |
| | component in NP | tuned aqueous-based |
| Light + thermal + | | Essentially thermally-enchanced |
| chemical | | light + chemical to speed degradation |

After dissociation of the nanoparticles or liposomes, the signal inducing agent entrapped therein can be released, preferably in a solution in which the signal inducing agent, as well as other components for the reaction involving the signal inducing agent as described herein, is soluble. The solution can be a homogenous solvent or a mixture of one or more solvent and/or one or more solutes. When a chemical trigger (e.g., an acid or a base) is used to dissociate the nanoparticles or liposomes, the chemical trigger can be placed in the solution.

The signal inducing agent released into the solution is then subject to a reaction to produce a product that is capable of releasing a detectable signal. The reaction can be any event that changes the physical or chemical property of one molecule (which can be the signal inducing agent itself), resulting in a signal change as described herein. In some embodiments, components required for occurrence of the reaction, e.g., substrates of a catalyst, fluorophore precursors, oxidant, reductant, pH modulators, substances to enhance the reaction or signal detection, as described herein may be contained in the same solution. Thus, the step of releasing the signal inducing agent from the nanoparticles or liposomes and the step of subjecting the agent to a reaction to produce a detectable signal can take place simultaneously. The signal change can be determined by a conventional method, e.g., an optical method or an electrical method. Based on the signal change, the presence or quantify of the analyte of interest can be measured.

In some embodiments the signal-inducing agent reacts physically or chemically to produce an electrical signal. In an example embodiment, the signal-inducing agent is a transition metal catalyst. In another embodiment, the transition metal catalyst reacts chemically with another substrate. In an example embodiment, the chemical reaction is an oxidation reaction. The oxidation potential of the oxidation reaction can produce an electrical or electrochemical signal which can be measured by methods known to those of skill in the art. The signal-inducing agent released from the nanoparticles or liposomes may remain free in solution during electrical interrogation.

Alternatively, the signal-inducing agent released from the nanoparticles or liposomes may comprise one or more functional moieties capable of binding to specific groups on or in the vicinity of an electrode. Electrode functionalization may be performed by multiple methods known to those skilled in the art including, but not limited to, electropolymerization, self-assembly, film adhesion, etc. Redox transfer agents, such as ferrocene, may also be present.

(ii) Competition Assays

In some embodiments, the assay methods described herein are carried out through competitive binding, which is suitable for, e.g., detecting small analytes.

The competitive assay may be performed by incubating a sample suspected of containing an analyte of interest with a binding agent specific to the analyte to form a binding agent/analyte complex, the binding agent being conjugated to the nanoparticles as described herein. Typically, the binding agent-nanoparticle conjugate or the binding agent-liposome conjugate is in excessive amount relative to the analyte in the sample. The more analyte in the sample, the less unbound nanoparticle remains. Thus, the amount of the unbound nanoparticle or liposome is inversely proportional to the amount of the analyte in the sample. The mixture is then incubated with a solid support on which the analyte is immobilized under conditions allowing for the binding of the unbound nanoparticle or liposome to the immobilized analyte. The solid support can be washed after the incubation to remove unbound substances. The nanoparticle or liposome that is bound to the solid support is then dissociated as described herein to release the signal inducing agent contained therein following the descriptions provided herein. The signal inducing agent can then be subject to a reaction as described herein to produce a signal change, based on which the presence and/or quantity of the analyte in the sample.

Alternatively, a competitive assay can be performed as follows. A sample suspected of containing an analyte of interest is incubated with a solid support under conditions allowing for immobilization of the analyte onto the solid support. The solid support is washed for multiple times to remove unbound substances and is then incubated with both a free binding agent specific to the analyte and nanoparticles as described herein, on which a binding agent specific to the analyte is attached. The binding agent attached to the nanoparticles or liposomes may be the same as the free binding agent. The free binding agent and the nanoparticle or liposome compete against each other for binding to the analyte immobilized on the solid support. After being washed for multiple times to remove any unbound substances, the nanoparticles or liposomes bound to the solid support can be dissociated following methods described herein to release the signal inducing agent contained in the nanoparticles. The signal inducing agent can then be subject to a reaction as described herein to produce a signal change, based on which the presence and/or quantity of the analyte in the sample.

In other embodiments, a competitive assay may comprise nanoparticles or liposomes on which an analyte of interest or a member of a receptor/ligand pair (e.g., biotin) is attached. To perform the assay method, a solid support on which a binding agent such as an antibody that is specific to the analyte is immobilized is provided. The solid support is incubated with a sample suspected of containing an analyte of interest in the presence of the nanoparticle or liposome on which the analyte is attached. The incubation is carried out under suitable conditions allowing for binding of the binding agent on the solid support to the analyte in the sample and that on the nanoparticles or liposomes. The analyte attached to the nanoparticle or liposome competes against the free analyte in the sample for binding to the binding agent on the solid support. After the incubation, the solid support can be washed for multiple times to remove unbound substances. The nanoparticles bound to the solid support can be dissociated following methods described herein to release the signal inducing agent contained in the nanoparticles or liposomes. The signal inducing agent can then be subject to a reaction as described herein to produce a signal change, based on which the presence and/or quantity of the analyte in the sample.

Alternatively, the solid support can be incubated with a sample suspected of containing the analyte of interest and a conjugate comprising the analyte and a member of a receptor/binding pair (e.g., biotin or streptavidin) under suitable conditions allowing for the formation of binding agent/analyte complex. The solid support can be washed for multiple times to remove unbound substances and then be incubated with nanoparticles or liposomes on which an agent that binds (directly or indirectly) the analyte conjugate is attached. For example, both the analyte conjugate and the nanoparticle or liposome may be biotinylated and the incubation is carried out in the presence of streptavidin, which bridges the binding of the analyte conjugate and the nanoparticle or liposome. After the incubation, the solid support is again washed for multiple times to remove unbound substances. The nanoparticles or liposomes bound to the solid support can be dissociated following methods described herein to release the signal inducing agent contained in the nanoparticles or liposomes. The signal inducing agent can then be subject to a reaction as described herein to produce a signal change, based on which the presence and/or quantity of the analyte in the sample.

(iii) Assays in Other Formats

In addition to Sandwich and competitive formats, the assay methods described herein can also be performed in other formats as known in the art or disclosed herein.

For example, the assay methods may be performed in a manner similar to direct ELISA as follows. A sample suspected of containing an analyte of interest can be incubated with a solid support under conditions allowing for the immobilization of the analyte onto the solid support. After being washed for several times to remove unbound substances, the solid support is incubated with a nanoparticle or liposome as described herein on which a binding agent specific to the analyte is attached to allow for binding of the nanoparticle or liposome (via the binding agent) to the immobilized analyte. The solid support is then washed again to remove unbound substances. The nanoparticles or liposomes bound to the solid support can be dissociated following methods described herein to release the signal inducing agent contained in the nanoparticles. The signal inducing agent can then be subject to a reaction as described herein to produce a signal change, based on which the presence and/or quantity of the analyte in the sample.

In another example, the assay method may be performed in a lateral flow assay format. Such an assay may be carried out on a solid support (e.g., a membrane). The solid support may be made by a suitable material that allows for movement of biomolecules along the solid support. Examples include, but are not limited to, nitrocellulose, nylon, cellulose, polyvinylidine fluoride (PVDF), polycarbonate, polypropylene, polyethylene, Teflon, and Kevlar. To perform the assay, a sample suspected of containing an analyte of interest can be placed at one end of the solid support. Upon moving along the solid support, the analyte in the sample binds any of the nanoparticles or liposomes as described herein to form a complex. The complex can then be captured by a capture agent which is immobilized at a specific zone of the solid support. Upon washing, the nanoparticles or liposomes are dissociated as described herein to release the signal inducing agent contained therein. Reagents needed for signal generating mediated by the signal inducing agent can be entrapped in microparticles, which are immobilized either at the zone as the capture agent or at a nearby zone such that the signal inducing agent, upon release, can enter into the microparticles for signal production. The reagents contained in the microparticles depend on the signal inducing agent used in the nanoparticles or liposomes. For example, if the signal inducing agent is a catalyst, a suitable substrate, as well as other relevant components as described herein, can be contained in the microparticles or liposomes.

In some embodiments, nanoparticles containing two different signal inducing agents are used for detecting/quantifying an analyte of interest in samples in different concentrations so as to obtain accurate results. For example, nanoparticles or liposomes containing a catalyst and a fluorophore as the signal inducing agents can be used. When a sample contains a low amount of the analyte, signal amplification may be needed. In that case, the catalyst is used to amplify the signal for detecting the analyte following the procedures described herein. On the other hand, if the sample contains a relative high amount of the analyte, the fluorophore can be used for detecting/quantifying the analyte in the sample.

In other embodiments, an assay method described herein involves the use of two or more nanoparticles or liposomes for detecting/quantifying two or more analytes in a sample. The two or more nanoparticles or liposomes are conjugated to binding agents targeting different analytes of interest. Further, the two or more nanoparticles or liposomes contain signal inducing agents, which upon reactions, produce different signals (e.g., green fluorescence or red fluorescence), which can be relied on for detecting or quantifying different analytes. In some examples, the two or more nanoparticles or liposomes can be made by the same or similar materials such that they can be dissociated by the same trigger (e.g., a physical trigger or chemical trigger).

### VII. KIT FOR PERFORMING ASSAY METHODS DESCRIBED HEREIN

The present disclosure also provides kits for use in performing the assay methods described herein. Such kits can include one or more conjugates each comprising a nanoparticle or liposome as described herein. The kit may further comprise components for performing a reaction in the presence of the signal inducing agent to produce a product, which results in a signal change. In some embodiments, the kit may comprise two or more nanoparticles or liposomes comprising different signal inducing agents and binding agents specific to different analytes.

The kit disclosed herein may further comprise relevant components in connection with the different assay format as described herein. For example, a kit for performing the assay method in Sandwich format may further comprise a binding agent specific to the same analyte as the binding agent attached to the nanoparticles or liposomes. The binding agent may be in free form or immobilized on a solid support. The binding agent and that attached to the nanoparticles or liposomes may bind to different epitopes of the same analyte.

In other embodiments, kit for performing the assay method in competitive assay format may further comprise a binding agent specific to the analyte, wherein the binding agent is either in free form or immobilized on a solid support and a conjugate comprising the analyte and molecule that can bind the nanoparticles or liposomes. Alternatively, the kit may further comprise the binding agent in free form and optionally a solid support for immobilizing the analyte in the sample. The free binding agent may be the same as the binding agent on the nanoparticle or liposome or may compete against the binding agent on the nanoparticle or liposome for binding to the analyte. In another example, the kit may further comprise the analyte either in free form or immobilized on a solid support.

In yet other embodiments, the kit comprises a membrane suitable for a lateral flow assay, on which necessary components (e.g., those described herein) are immobilized.

In some embodiments, the kit can further comprise instructions for use in accordance with any of the methods described herein. The included instructions can comprise a description of performing each step of the the assay method. The kit may further comprise a description of selecting suitable samples to be analyzed by the assay method.

The kits provided herein are in suitable packaging. Suitable packaging includes, but is not limited to, vials, bottles, jars, flexible packaging (e.g., sealed Mylar or plastic bags), and the like. Also contemplated are packages for use in combination with a specific device, including any spectrometer, fluorescence spectrophotometer, and/or luminometer. These include 6-, 12-, 48-, 96-, 384-well benchtop microplate readers offered by multiple vendors (e.g. Perkin-Elmer, Molecular Devices), benchtop devices (e.g. Abbott, Alere, BioMerieur), automated devices (e.g. Siemens, Roche).

Kits may optionally provide additional components such as buffers and interpretive information. Normally, the kit comprises a container and a label or package insert(s) on or associated with the container. In some embodiments, the present disclosure provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### VIII. EXAMPLES

### Example 1: Fe(III)-TAML Encapsulated Nanoparticles with FL-DL Matrix

Materials:
Fe(III)-TAML® (sodium salt) metalorganic compound purchased from GreenOx (see, e.g., U.S. Pat. No. 6,100,394, which is incorporated herein by reference in its entirety) having the following structure,

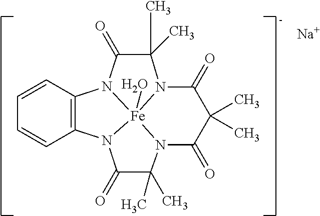

Adogen 464 (Sigma Aldrich)
Fluorescein-Dilaurate (FL-DL) (Sigma Aldrich)
DSPE-PEG-2k-Biotin (Lysan Bio)
Benzyl Alcohol (BA) (Sigma)
Ethyl Acetate (EA) (Sigma)
De-Ionized (DI) Water

Nanoparticles comprising a Fe(III)-TAML metalorganic compound were prepared according to the following procedure. See also Table 3.
1. 40 gm of DI Water were weighed in a beaker. 1.6 gm of BA were added to the beaker and stirred on a magnetic stirrer until the BA has dissolved in the DI water.
2. 2 g of BA were weighed into a 20 mLs scintillation glass vial. 5 mgs of TAML and 20 mgs of Adogen 464 were added to the BA solution, which were dissolved by vortexing.
3. 20 mg of FL-DL and 1.0 mg of DSPE-PEG-2k-Biotin were added to the above mixture and also dissolved by vortexing.
4. 3 g of EA were added and mixed.
5. The contents of the organic mixture in step 4 were added to the aqueous solution (with BA) of Step 1.
6. A coarse emulsion was obtained by emulsifying with a hand held homogenizer.
7. The coarse emulsion of Step 6 was passed through a Microfluidizer at a pressure of 7000 psi to make a fine emulsion.
8. The fine emulsion was quenched by adding it to a beaker with 200 gms of DI water with stirring to obtain the nanoparticles.
9. Tangential flow filtration (TFF) was used on the above nanoparticle solution of Step 8 and then concentrated to 20 mLs.
10. 00 mLS of PBST (0.05% Tween 80) was added, and the mixture was concentrated by TFF to 20 mLs.
11. The nanoparticles were collected in a 20 mLs glass scintillation vial.
12. The nanoparticles were filtered through a 0.2 μm filter to give the final product.

TABLE 3
SL-135 Composition

| Component | Mol % |
|---|---|
| TAML | 14 |
| Adogen 464 | 28 |
| FL-DL | 57 |
| DSPE-PEG-Biotin-2k | 1 |

A polydispersity index of 0.078 for the SL-135 composition was measured.

### Example 2: Performance of Nanoparticle with Encapsulated Fluorescein Dilaureate and TAML Catalyst

One lot of nanoparticles (SL131) was prepared with encapsulated fluorescein dilaurate and TAML catalyst, along with biotinylated surface. A 1:3 seral dilutions of this particle are prepared and 100 μL per well of each diluted solution was added to a commercial streptavidin coated 96-well microtiter plate and incubated at room temperature shaking at 575 rpm for 60 min, to allow the biotinylated particles to bind to the streptavidin on the well bottom. The plate was then washed with 350 μL PBST per well for a total of 4 times to remove non-bound nanoparticle from the well. Upon binding, a SA-biotin-nanoparticle complex is formed.

The presence of the specifically bound nanoparticles was visualized by addition of 150 μL ethanol and 50 μL of 1M NaOH per well. Ethanol disrupts the nanoparticle to release the fluorescen dilaurate molecules and NaOH frees the fluorescein from the non-fluorescent fluorescein dilaurate by breaking down the ester bond between the fluorescein and the dilaureats moieties. The fluorescent signal was collected by reading the plate at excitation/emission/cutoff of 490 nm/545 nm/530 nm, which is specific for the fluorescein.

The activity of the encapsulated TAML catalyst was tested using the on fluorescent Amplex Red as enzyme substrate. In the presence of H₂O₂, TAML catalyzes the oxidation of non-fluorescent Amplex Red to the strongly fluorescent resorufin with an Excitation/emission of 530/590 nm. To each well with SA-biotin captured nanoparticle, the following reagents were added −50 μL of 200 μm Amplex Red, 150 μL of carbonate-bicarbonate buffer, (pH10.01), 50 μL of 1 mM H₂O₂. After a brief mixing by gently tapping the plate and a 15 minute incubation at room temperature, the fluorescent signal was collected by reading the plate at Ex530/Em590/Cutoff590.

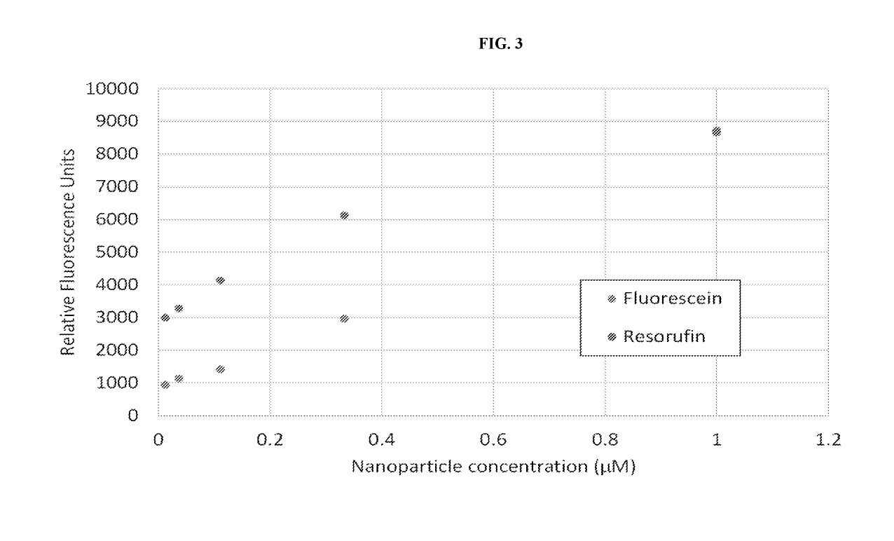

As shown in FIG. 3, the fluorescent signals of both fluorescein dilaurate dependent fluorescein and TAML-dependent resorufin are correlated with the concentration of S1131 nanoparticle.

### Example 3: Stability of TAML-Loaded Nanoparticles and Compatibility with Immunoassays

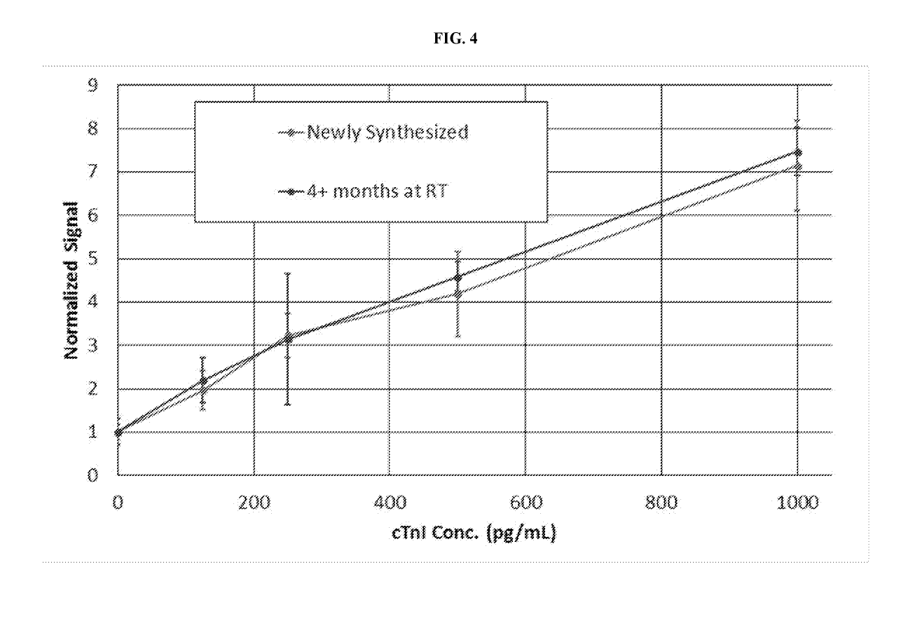

FIG. 4 is a plot illustrating the signal strength of newly synthesized nanoparticles compared to nanoparticles stored at room temperature for four months for use in a cTnl ELISA assay. Poly(lactic acid)-diacrylate (20 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-2000](DSPE-PEG-biotin; Laysan Bio) was then added and the solution was heated to 50° C. A solution of Compritol 888 CG ATO (Gattefosse) in benzene at 50° C. was added. This solution was homogenized into a 5× volume of deionized water at 50° C., forming a stable, milky emulsion with water as the continuous phase. The emulsion, in an open beaker, was transferred to a room-temperature hotplate and was left stirring overnight in a chemical fume hood to enable solvent evaporation. The resulting particles were filtered and concentrated with a 300 kD membrane (EMD Merck) and measured by the NanoSight (Malvern) technique. The average particle size was 115 nm and the standard deviation was 35 nm.

In order to confer neutravidin functionality to the particle surface, a particle concentration was determined such that the effective biotin concentration in 1× phosphate buffered saline, pH ˜7.2 was 0.1 μm. Neutravidin (ThermoFisher) was added at a concentration of ˜10 μm and binding was allowed to proceed at room temperature for 2 hours. The resulting particles were filtered and concentrated with a 300 kD membrane (EMD Merck) and NanoSight measurements showed a slight increase in particle size to 120 nm and standard deviation, 45 nm.

The particles were then used as the reporters in a human cardiac troponin (cTnI) immunoassay. A human cTnI ELISA microplate kit (Ray Biotech) was used and a standard curve was prepared as instructed. The neutravidin-functionalized, TAML-loaded nanoparticles were used in place of the avidin-horseradish peroxidase enzyme during the final binding step. The particles were burst and a solution of hydrogen peroxide and 2,7-dihydrodichlorofluorescein diacetate at pH 10 was then added and the fluorescence at 490/545 was measured. The results of freshly-made nanoparticles were compared with particles stored for 2 months at 37° C.

### Example 4: TAML-Loaded Nanoparticle Application to DNA Hybridization Assay

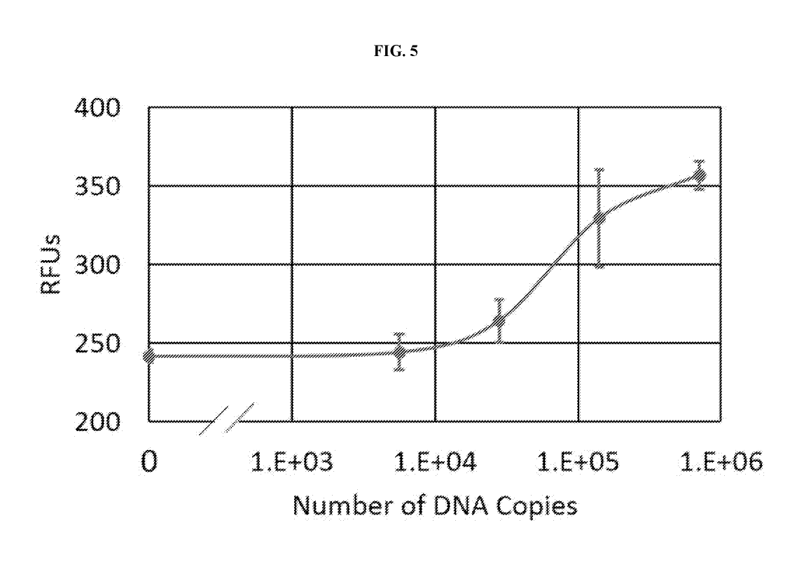

FIG. 5 is a plot illustrating the reflective fluorescence unit intensity (RFU) as compared to the number of DNA copies made in a DNA-hybridization assay. Poly(lactic acid)-diacrylate (20 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[maleimidely(polyethylene glycol)-2000] (DSPE-PEG-maleimide; Laysan Bio) was then added in addition to 2,2′-azosisobutyronitrile (AIBN), styrene, and divinyl benzene. This solution was homogenized into a 5× volume of deionized water, forming a stable, milky emulsion with water as the continuous phase. The suspension was loaded into a round-bottom flask fitted with a reflux condenser, flushed with nitrogen, heated to 50° C. and stirred at 400 rpm such that a vortex formed in the flask. The reaction proceeded for 2 hours, after which the heat was removed and the suspension was decanted into an open beaker. Thiol-terminated DNA oligonucleotides (Integrated DNA Technologies, IDT; 5′-THIOL-spacer18-AGAATAGTTTTATGGGATTAG-3′) were introduced together with a 100-molar excess of tris(2-carboxyethyl)phosphine and the pH was adjusted to 6.5. The solution was stirred overnight in a chemical fume hood to enable solvent evaporation. The resulting particles were filtered and concentrated with a 300 kD membrane (EMD Merck) and stored in 1×SSC buffer.

An assay for Listeria Monocytogenes DNA was developing by using the primer-functionalized nanoparticles together with a second primer functionalized to magnetic microparticles. Amino-reactive magnetic microparticles with 2 m diameters (BioClone) were reacted with amine-terminated DNA oligonucleotides (IDT; 5′-CTATCCATTGTAGCACGTG-spacer18-amino-3′) overnight at 50° C. according to the manufacturer's instructions. A hybridization reaction was performed with purified L. Monocytogenes genomic material (American Type Culture Center) dissolved in a 1×SSC buffer containing 1% bovine serum albumin and 1% salmon sperm DNA (Sigma-Alrich). The reaction proceeded for 30 minutes at 45° C., after which time the magnetic particles were thoroughly washed using a magnetic stand (Promega). The particles were burst and a solution of hydrogen peroxide and 2,7-dihydrodichlorofluorescein diacetate at pH 10 was then added and the fluorescence at 490/545 was measured. The fluorescent signals of a serial dilution of samples are compared against the known quantities of genomic material determined by quantitative PCR.

### Example 5: Use of Nanoparticles in a Sandwich Immunoassay Compared to Use of Enzyme in Sandwich Immunoassay

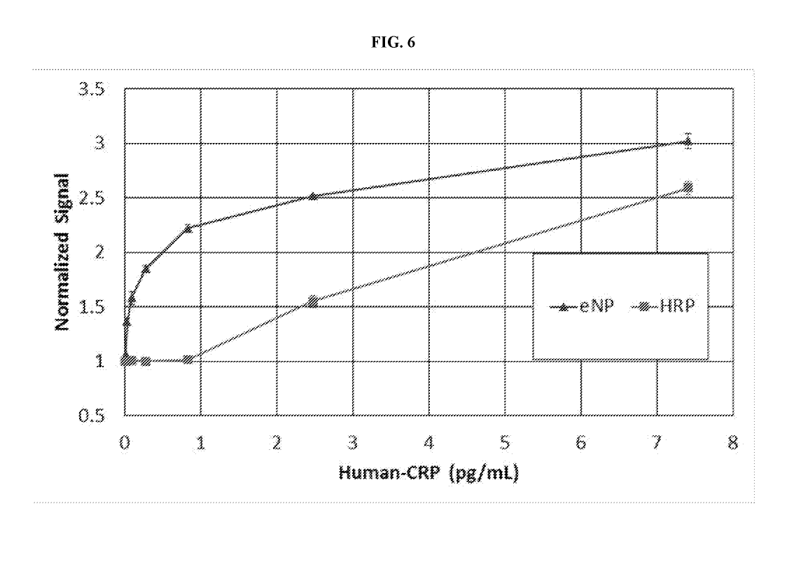

FIG. 6 is a plot illustrating the normalized optical signal from a sandwich immunoassay for human C-reactive protein (CRP). The nanoparticle (eNP)-based assay output a fluorescent signal and the enzyme (HRP)-based assay output an optical signal, thus values are normalized for each to the zero-concentration point.

Poly(lactic acid)-diacrylate (20 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-2000](DSPE-PEG-biotin; Laysan Bio) was then added in addition to 2,2′-azosisobutyronitrile (AIBN), styrene, and divinyl benzene. This solution was homogenized into a 5× volume of deionized water, forming a stable, milky emulsion with water as the continuous phase. The suspension was loaded into a round-bottom flask fitted with a reflux condenser, flushed with nitrogen, heated to 50° C. and stirred at 400 rpm such that a vortex formed in the flask. The reaction proceeded for 2 hours, after which the heat was removed and the suspension was decanted into an open beaker. The solution was stirred overnight in a chemical fume hood to enable solvent evaporation. The resulting particles were filtered and concentrated in 1×PBS with a 300 kD membrane (EMD Merck).

In order to confer neutravidin functionality to the particle surface, a particle concentration was determined such that the effective biotin concentration in 1×PBS, pH ˜7.2 was 0.1 μm. Neutravidin (ThermoFisher) was added at a concentration of ˜10 μm and binding was allowed to proceed at room temperature for 2 hours. The resulting particles were filtered and concentrated with a 300 kD membrane (EMD Merck) and stored in 1×PBS.

The particles were then used as the reporters in a human CRP immunoassay and compared against an enzyme reporter, HRP. A capture monoclonal antibody for human CRP (Abcam) was bound to N-hydroxysuccinimyl (NHS)-ester activated magnetic beads (ThermoFisher). The particles were washed with ice-cold 1 mM hydrochloric acid and the coupling was performed for 2 hours at room temperature with a 50 mM borate buffer, pH 8.0. A detection monoclonal antibody (Abcam) was biotinylated using sulfo-NHS-LC-biotin 888(ThermoFisher). The coupling reaction was performed for 2 hours at room temperature in 1×PBS, pH 7.5. The human CRP protein was used from a commercial CRP ELISA microplate kit (Abcam) and a standard curve was prepared in ⅓-dilutions from 600 pg/mL to 0.01 pg/mL.

The immunoassay steps were performed in triplicate in polypropylene tubes and a magnetic stand (Promega) was used for bead immobilization during wash steps. After the wash following detection antibody binding, the volume of each tube was split in two and either streptavidin-HRP (Abcam) or neutravidin-functionalized, MTALC-loaded nanoparticles. The streptavidin-HRP labeled assays were developed with TMB solution (Abcam), stopped with dilute sulfuric acid (Abcam), and the absorbance was read at 450 nm. The nanoparticles were burst with acetone and a solution of hydrogen peroxide and 2,7-dihydrodichlorofluorescein diacetate at pH 10 was then added and the fluorescence at 490/545 was measured.

### Example 6: Synthesis of Nanoparticles Comprising Transition Metal Catalysts

### Example 6.1

Poly(lactic acid)-diacrylate (20 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-2000](DSPE-PEG-biotin; Laysan Bio) was then added. This solution was homogenized into a 5× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.2

Poly(lactic acid)-diacrylate (20 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-2000](DSPE-PEG-biotin; Laysan Bio) was then added together with poly(lactic acid) (20 kD; PolySciTech). This solution was homogenized into a 5× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.3

Poly(lactic acid)-diacrylate (20 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-2000](DSPE-PEG-biotin; Laysan Bio) was then added in addition to 2,2′-azosisobutyronitrile (AIBN), styrene, and divinyl benzene. This solution was homogenized into a 5× volume of deionized water, forming a stable, milky emulsion with water as the continuous phase. The suspension was loaded into a round-bottom flask fitted with a reflux condenser, flushed with nitrogen, heated to 50° C. and stirred at 400 rpm such that a vortex formed in the flask. The reaction proceeded for 2 hours, after which the heat was removed and the suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.4

Poly(lactic acid)-acid (˜180 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-3400](DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.5

Poly(lactic acid)-acid (15-25 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-3400](DSPE-PEG-biotin; Laysan Bio), along with fluorescein dilaurate (FDL; Sigma Aldrich) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.6

Poly(lactic acid)-diacrylate (20 kD; PolySciTech) and the MTALC iron salt (GreenOx Catalysts), in a 5:1 mass ratio, were dissolved in an acetonitrile solution containing pentaerythritol tetrakis(3-mercaptopropionate) (PT3MP; Sigma Aldrich) and 2,2-dimethoxy-2-phenylacetophenone (DMPA Sigma). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The resulting solution was subjected to long wave UV irradiation to facilitate the thiolene-click reaction between PT3MP and acrylate species on the poly(lactic acid) polymer. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.7

Poly(lactic acid)-diacrylate (20 kD; PolySciTech) and the MTALC iron salt (GreenOx Catalysts), in a 5:1 mass ratio, were dissolved in an acetonitrile solution containing pentaerythritol tetrakis(3-mercaptopropionate) (PT3MP; Sigma Aldrich) and 2,2-dimethoxy-2-phenylacetophenone (DMPA Sigma). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP and acrylate species on the poly(lactic acid) polymer. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) and 1,2-distearoyl-sn-glycero-3-phosphoethanolamine-N-[amine(polyethylene glycol)-2000] (DSPE-PEG-amine; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.8

Poly(lactic acid)-acid (15-25 kD; PolySciTech) and the MTALC iron salt (GreenOx Catalysts) were dissolved in acetonitrile in a 5:1 mass ratio. A solution of polystyrene-block-polyisoprene-block-polystyrene (PS-PI-PS) in heptane was prepared and the acetonitrile solution was added and homogenized into the heptane at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-3400](DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.9

Pentaerythritol tetrakis(3-mercaptopropionate) (PT3MP; Sigma Aldrich) and the MTALC iron salt (GreenOx Catalysts), in a 5:1 mass ratio, were dissolved in an acetonitrile solution containing 2,2-dimethoxy-2-phenylacetophenone (DMPA; Sigma Aldrich), along with styrene (Sigma Aldrich), divinlybenzene (Sigma Aldrich), or a mixture of the two. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The resulting solution was subjected to long wave UV irradiation to facilitate the thiolene-click reaction between PT3MP and divinylbenzene. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.10

Subsequent washes of particles were performed to determine the amount of MTALC bound within the particle compared with that associated with the particle. The particles were loaded into a 300 kD-cutoff microfuge spin-filter column (VWR) and washes were performed with PBST and filtrates were collected. MTALC concentrations were determined fluorescent with the addition of 0.1 M sodium bicarbonate buffer (pH ˜10) containing 30 μM hydrogen peroxide and 600 μM DCFH-DA. Particles were resuspended in PBST for each wash by pipetting up-and-down five times. The final MTALC determination was made by first introducing acetone to the filter and pipetting up-and-down, followed by the addition of 0.1 M sodium bicarbonate buffer (pH ˜10), followed by centrifugation and collection. Standard curves were established with soluble MTALC for quantification and particle loading was determined using a NanoSight to measure particle concentration.

### Example 6.11

Poly(lactic acid)-diacrylate (20 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-2000](DSPE-PEG-biotin; Laysan Bio) was then added in addition to 2,2′-azosisobutyronitrile (AIBN), styrene, and 1,4-phenylene dimethacrylate. This solution was homogenized into a 5× volume of deionized water, forming a stable, milky emulsion with water as the continuous phase. The suspension was loaded into a round-bottom flask fitted with a reflux condenser, flushed with nitrogen, heated to 50° C. and stirred at 400 rpm such that a vortex formed in the flask. The reaction proceeded for 2 hours, after which the heat was removed and the suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.12

Poly(lactic acid)-diacrylate (20 kD; PolySciTech) and the MTALC iron salt (GreenOx Catalysts) were dissolved in acetonitrile in a 5:1 mass ratio. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-2000](DSPE-PEG-biotin; Laysan Bio) was then added in addition to 2-cyano-2-propyl dodecyl trithiocarbonate, 2,2′-azosisobutyronitrile (AIBN), styrene, and 1,4-phenylene dimethacrylate. This solution was homogenized into a 5× volume of deionized water, forming a stable, milky emulsion with water as the continuous phase. The suspension was loaded into a round-bottom flask fitted with a reflux condenser, flushed with nitrogen, heated to 50° C. and stirred at 400 rpm such that a vortex formed in the flask. The reaction proceeded for 16 hours, after which the heat was removed and the suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.13

Poly(lactic acid)-diamine (20 kD; PolySciTech) and the MTALC iron salt (GreenOx Catalysts), in a 5:1 mass ratio, were dissolved in acetonitrile. A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. Benzene was then added to clarify the suspension. 1,2-distearoyl-sn-glycero-3-phosphoethanolamine-N-[biotinyl(polyethylene glycol)-3400](DSPE-PEG-biotin; Laysan Bio) was then added. This solution was homogenized into an 8× volume of deionized water, while adding a solution of compritol 888 (Sigma Aldrich) in benzene, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.14

Poly(lactic acid)-diamine (20 kD; PolySciTech) and the MTALC iron salt (GreenOx Catalysts), in a 5:1 mass ratio, were dissolved in acetonitrile, along with poly(vinyl alcohol), N-methyl-4(4′-formylstyryl)pyridinium methosulfate acetal (PVA-SbQ; PolySciTech). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was then irradiated with UV light to facilitate the photocrosslinking of the PVA-SbQ. Benzene was then added to clarify the suspension. 1,2-distearoyl-sn-glycero-3-phosphoethanolamine-N-[biotinyl(polvethvlene glycol)-34001 (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.15

The MTALC iron salt (GreenOx Catalysts) was dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.16

The MTALC iron salt (GreenOx Catalysts) was dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), while adding tris(2-aminoethyl)amine (TAEA; Sigma Aldrich), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400](DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.17

The MTALC iron salt (GreenOx Catalysts) was dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), while adding 2,2′-(ethylenedioxy)bis(ethylamine) (22EBE); Sigma Aldrich), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.18

The MTALC iron salt (GreenOx Catalysts) was dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), while adding Adogen 464 (A464; Sigma Aldrich), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.19

The MTALC iron salt (GreenOx Catalysts) was dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), while adding Adogen 464 (A464; Sigma Aldrich), 2,2′, (ethylenedioxy)bis(ethylamine) (22EBE); Sigma Aldrich), and tris(2-aminoethyl)amine (TAEA; Sigma Aldrich), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400](DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.20

Poly(lactic acid)-diacrylate (20 kD; PolySciTech) and the MTALC iron salt (GreenOx Catalysts), in a 5:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.21

Poly(lactic acid)-diamine (20 kD; PolySciTech) and the MTALC iron salt (GreenOx Catalysts), in a 5:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.22

Poly(lactic acid)-diacrylate (20 kD; PolySciTech) and the MTALC iron salt (GreenOx Catalysts), in a 5:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), while adding tris(2-aminoethyl)amine (TAEA; Sigma Aldrich), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.23

Poly(lactic acid)-diacrylate (20 kD; PolySciTech) and the MTALC iron salt (GreenOx Catalysts), in a 5:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), while adding 2,2′-(ethylenedioxy)bis(ethylamine) (22EBE); Sigma Aldrich), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.24

Poly(lactic acid)-diacrylate (20 kD; PolySciTech) and the MTALC iron salt (GreenOx Catalysts), in a 5:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), while adding Adogen 464 (A464; Sigma Aldrich), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.25

Poly(lactic acid)-diamine (20 kD; PolySciTech) and the MTALC iron salt (GreenOx Catalysts), in a 5:1 mass ratio, were dissolved in acetonitrile. A solution of poly(maleic anhydride-alt-octadecane) (PMAOD) in cyclohexanes was prepared and the acetonitrile solution was added and homogenized into the cyclohexanes at 7,500 rpm (IKA), while adding tris(2-aminoethyl)amine (TAEA; Sigma Aldrich), forming a stable, milky emulsion. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.26

Poly(lactic acid)-diamine (20 kD; PolySciTech) and the MTALC iron salt (GreenOx Catalysts), in a 5:1 mass ratio, were dissolved in acetonitrile. A solution of poly(maleic anhydride-alt-octadecane) (PMAOD) in cyclohexanes was prepared and the acetonitrile solution was added and homogenized into the cyclohexanes at 7,500 rpm (IKA), while adding 2,2′, (ethylenedioxy)bis(ethylamine) (22EBE); Sigma Aldrich), forming a stable, milky emulsion. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.27

Poly(lactic acid)-diamine (20 kD; PolySciTech) and the MTALC iron salt (GreenOx Catalysts), in a 5:1 mass ratio, were dissolved in acetonitrile. A solution of poly(maleic anhydride-alt-octadecane) (PMAOD) in cyclohexanes was prepared and the acetonitrile solution was added and homogenized into the cyclohexanes at 7,500 rpm (IKA), while adding Adogen 464 (A464; Sigma Aldrich), 2,2′, (ethylenedioxy)bis(ethylamine) (22EBE); Sigma Aldrich), and tris(2-aminoethyl)amine (TAEA; Sigma Aldrich), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin: Lavsan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.28

Amine-Poly(Ethylene Glycol)-Thiol (NH-PEG-SH; 1 kD; Laysan Bio. Inc.) and the MTALC iron salt (GreenOx Catalysts), in a 2:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP, PTTA, and NH-PEG-SH. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.29

Amine-Poly(Ethylene Glycol)-Thiol (NH-PEG-SH; 1 kD; Laysan Bio. Inc.) and the MTALC iron salt (GreenOx Catalysts), in a 2:1 mass ratio, were dissolved in an acetonitrile solution containing 2,2-dimethoxy-2-phenylacetophenone (DMPA; Sigma Aldrich) and Adogen 464 (A464; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP, PTTA, and NH-PEG-SH. Benzene was then added to clarify the suspension. 1,2-distearoyl-sn-glycero-3-phosphoethanolamine-N-[biotinyl(polyethylene glycol)-3400](DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.30

Amine-Poly(Ethylene Glycol)-Amine (NH-PEG-NH; 2 kD; Laysan Bio. Inc.) and the MTALC iron salt (GreenOx Catalysts), in a 2:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.31

Amine-Poly(Ethylene Glycol)-Amine (NH-PEG-NH; 2 kD; Laysan Bio. Inc.) and the MTALC iron salt (GreenOx Catalysts), in a 2:1 mass ratio, were dissolved in an acetonitrile solution containing 2,2-dimethoxy-2-phenylacetophenone (DMPA; Sigma Aldrich) and Adogen 464 (A464; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP and PTTA. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.32

Amine-Poly(Ethylene Glycol)-Thiol (NH-PEG-SH; 1 kD; Laysan Bio. Inc.), Amine-Poly(Ethylene Glycol)-Amine (NH-PEG-NH; 2 kD; Laysan Bio. Inc.), and the MTALC iron salt (GreenOx Catalysts), in a 1:1:0.5 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP, PTTA, and NH-PEG-SH. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.33

Amine-Poly(Ethylene Glycol)-Thiol (NH-PEG-SH; 1 kD; Laysan Bio. Inc.), Amine-Poly(Ethylene Glycol)-Amine (NH-PEG-NH; 2 kD; Laysan Bio. Inc.), and the MTALC iron salt (GreenOx Catalysts), in a 1:1:0.5 mass ratio, were dissolved in an acetonitrile solution containing 2,2-dimethoxy-2-phenylacetophenone (DMPA; Sigma Aldrich) and Adogen 464 (A464; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP, PTTA, and NH-PEG-SH. Benzene was then added to clarify the suspension. 1,2-distearoyl-sn-glycero-3-phosphoethanolamine-N-[biotinyl(polyethylene glycol)-3400](DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.34

1,2-dioleoyl-sn-glycero-3-phosphoethanolamine-N-[methoxy(polyethylene glycol)-1000] (ammonium salt) (1.8 kD; Avanti Polar Lipids, Inc.) and the MTALC iron salt (GreenOx Catalysts), in a 5:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), forming a stable, milky emulsion. The resulting solution was subjected to long wave UV irradiation to facilitate the thiolene-click reaction between PT3MP and PTTA and the alkenes within the 1,2-dioleoyl-sn-glycero-3-phosphoethanolamine-N-[methoxy(polyethylene glycol)-1000]. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 6.35

1,2-dioleoyl-sn-glycero-3-phosphoethanolamine-N-[methoxy(polyethylene glycol)-1000] (ammonium salt) (1.8 kD; Avanti Polar Lipids, Inc.) and the MTALC iron salt (GreenOx Catalysts), in a 5:1 mass ratio, were dissolved in an acetonitrile solution containing 2,2-dimethoxy-2-phenylacetophenone (DMPA; Sigma Aldrich), along with pentaerythritol tetrakis(3-mercaptopropionate) (PT3MP; Sigma Aldrich) and pentaerythritol tetraacrylate (PTTA; Sigma Aldrich). A solution of poly(maleic anhydride-alt-octadecane) (PMAOD) in cyclohexanes was prepared and the acetonitrile solution was added and homogenized into the cyclohexanes at 7,500 rpm (IKA), while adding 2,2′, (ethylenedioxy)bis(ethylamine) (22EBE); Sigma Aldrich), forming a stable, milky emulsion. The resulting solution was subjected to long wave UV irradiation to facilitate the thiolene-click reaction between PT3MP and PTTA and the alkenes within the 1,2-dioleoyl-sn-glycero-3-phosphoethanolamine-N-[methoxy(polyethylene glycol)-1000]. Benzene was then added to clarify the suspension. 1,2-distearoyl-sn-glycero-3-phosphoethanolamine-N-[biotinyl(polyethylene glycol)-3400] (DSPE-PEG-biotin; Laysan Bio) was then added. This solution was homogenized into an 8× volume of deionized water, forming a stable, milky emulsion with water as the continuous phase. The suspension was decanted into an open beaker, which was stirred overnight in a chemical fume hood to enable solvent evaporation. The resulting particles were filtered and concentrated with a 300 kD membrane (EMD Merck) and measured by DLS and NanoSight techniques.

### Example 7: Fluorescein Dilaurate (FL-DL) Particles

Materials:
Fluorescein-Dilaurate (FL-DL) (Sigma Aldrich);
DSPE-PEG-2k-Biotin (Lysan Bio);
Ethanol (Sigma); and
De-Ionized (DI) Water.

Fluorescein dilaurate (FL-DL) nanoparticles (NPs) (Table 4) were prepared according to the following procedure.
1. 20 mgs of FL-DL were weighed into a clear glass scintillation vial.
2. 1000 mgs of Ethanol were added, and the above FL-DL was dissolved by vortexing the vial.
3. 10 mgs of DSPE-PEG-2k-Biotin were added to the above mixture and dissolved by vortexing.
4. 40 gm of DI water were weighed out in a beaker, and a stir-bar was added. The beaker was placed on a magnetic stirrer and stirred at 200 RPM.
5. The Ethanol solution of Step 3 was added to the beaker in a drop wise fashion.
6. 200 gm of DI water was added to the mixture of Step 5.
7. Purify, and the volume of the mixture was concentrated to 20 mLs.
8. 100 gms of PBST (0.05% Tween 80) were added to the concentrate of Step 7.
9. Tangential Flow Filtration (TFF) was used for purification and concentration. Purified NPs were concentrated ˜12-fold to 20 mL and collected in a glass scintillation vial.
10. The above collected nanoparticle formulation of Step 9 was filtered through a 0.2 μm Filter.
11. Size was measured with a Dynamic Light Scattering instrument.

TABLE 4

| | | Mol % FL-DL |
|---|---|---|
| | Mol % DSPE-PEG-2k-Biotin | |
| SL-113 | 10 | 90 |
| SL-118 | 8 | 92 |
| SL-119 | 6 | 94 |
| SL-120 | 4 | 96 |
| | Mol % DSPE-PEG-5k-Biotin | |
| SL-123 | 6 | 94 |

A polydispersity index of 0.090 was measured for a composition (SL-135) comprising fluorescein-dilaurate, and DSPE-PEG-2k-Biotin.

### Example 8: Alternative Synthesis of Nanoparticles

Fluorescein dilaurate (Sigma Aldrich), 1,2-distearoyl-sn-glycero-3-phosphoethanolamine-N-[amino(polyethylene glycol)] (ammonium salt) (2K or 3.4K; Laysan Bio., Inc.), and 1,2-distearoyl-sn-glycero-3-phosphoethanolamine-N-[biotinyl(polyethylene glycol)] (ammonium salt) (2K or 3.4K; Laysan Bio., Inc.), were dissolved in a 1:1.5 solution of benzyl alcohol and ethyl acetate in varying mass ratios in a scintillation vial. The reaction solution was then stirred (and lightly heated) to dissolve. The organic solution was then added to an aqueous solution of 2% benzyl alcohol/4% ethyl acetate in DI at a 1:8 mass ratio. The resulting, phase separated solution was homogenized for ˜5 sec using a high shear mixer and then processed through a microfluidizer at 9500 psi to yield a highly monodisperse emulsification product which was immediately added to an excess of water to remove the organic solvents from the core of the formed nanoparticles, yielding stable nanoparticles loaded with fluorescein dilaurate as a fluorescent marker. The product solution was run through a tangential flow filtration system to concentrate the solution down to 20 mL before being filtered through a 0.45 μm syringe filter and characterized by both DLS and NanoSight measurements.

### Example 9: Non-Specific Binding of Nanoparticles

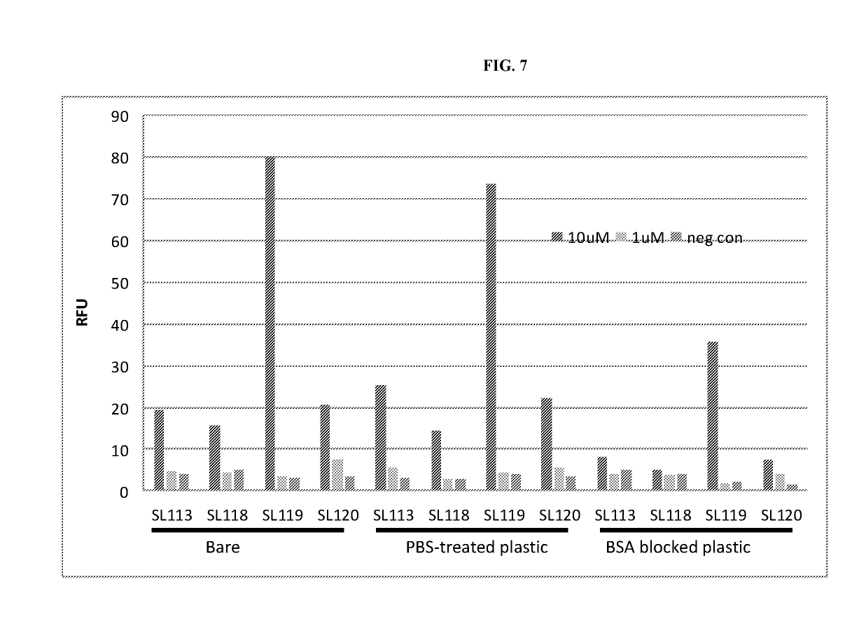

Four lots of nanoparticles (SL113, SL118, SL119 and SL120) were tested in this experiment for their non-specific binding activity to three surfaces, and these data are presented in FIG. 7. A Nunc MaxiSorp 96-well plate was used to generate three surfaces including: non-treated base plastic surface, plastic surface pretreated with PBS, and plastic surface pretreated with PBS containing 1% bovine serum albumin (BSA).

Each lot of nanoparticles was diluted in PBSTB 1 (phosphate buffered saline containing 0.05% Tween-20 and 1% BSA, pH 7.2) to prepare a nanoparticle solution containing 10 μM equivalent fluorescein dye and a nanoparticle solution containing 1 μM equivalent fluorescein dye. A total of 200 μL of 10 μM or 1 μM solution was added to each well in triplicate and the plate was incubated at room temperate for 60 minutes, while shaking at 575 rpm for efficient binding. For the no particle negative control, 200 μL per well of PBSTB 1 was used in duplicate. At the end of incubation, the non-bound nanoparticles were washed off by a total of 4 washes each with 350 μL PBST using an automatic plate washer. The residual liquid of each well was removed by gently tapping the plate (top down) onto clean tissue paper 3-5 times.

Then, 150 μL of pure ethanol was added to each well to disrupt the nanoparticle and 50 μL of 1M NaOH was added to each well to breakdown the ester bond between fluorescein dilaurate generating free fluorescein dye molecules. The fluorescence was read using a SpectraMax M2 plate reader with excitation/emission/cutoff setting at 490/545/530.

Overall, these nanoparticles showed very low non-specific binding activity to bare plastic surface and PBS-pretreated surface. Pretreatment of plastic surface with 1% BSA further reduced this non-specific binding. Comparatively speaking, lot SL119 showed slightly higher non-specific binding than other lots tested. In addition, the non-specific binding is nanoparticle concentration dependent.

### Example 10: Non-Specific Binding of Nanoparticles

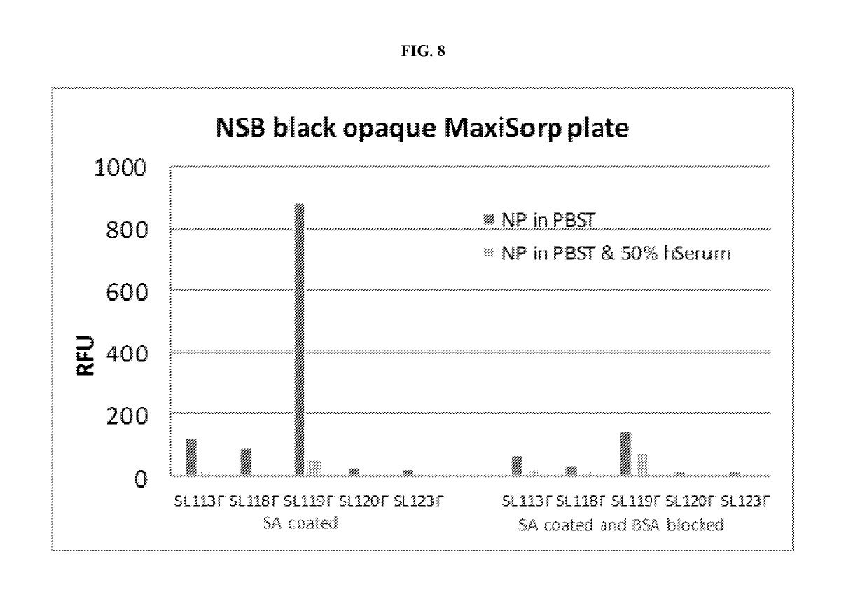

Five lots of nanoparticles (SL113, SL118, SL119, SL120 and SL123) were tested in this experiment for their non-specific binding activity to two surfaces, and these data are presented in FIG. 8.

A MaxiSorp 96-well plate was pretreated with 100 μL per well of 10 ug/mL streptavidin (SA) in PBS for 60 minutes at room temperature. The plate was washed with PBST for a total of four times. Then, half of the plate was treated with 100 μL per well of PBSB1 (PBS containing 1% BSA) for additional 60 minutes. After another 4× washing, the plate was used for the binding assay.

Two binding buffer matrices were used in this experiment: PBST (PBS containing 0.05% Tween-20) and PBST containing 33% human serum (PBST-33% hSerum). The nanoparticles were diluted in PBST or PBST-33% hSerum to 10 μM equivalent fluorescein dye concentration. A total of 200 μL per well of each diluted nanoparticle solution was added to the well coated with streptavidin or well coated with streptavidin and blocked with PBSB1. The plate was incubated at room temperate for 60 minutes, while shaking at 575 rpm. At the end of incubation, the non-bound nanoparticles were washed off by a total of 4 washes each with 350 μL PBST using an automatic plate washer. The residual liquid of each well was removed by gently tapping the plate (top down) onto clean tissue paper 3-5 times. The fluorescence signal was collected as described previously.

The nanoparticles (lots—SL119, SL120 and SL123) showed very low non-specific binding activity to SA coated surface and SA-BSA coated surface in both PBST and PBST containing 33% human serum. In PBST, SL119 showed much higher non-specific binding than other lots of nanoparticles tested in PBST matrix, addition of human serum into PBST significantly reduced this elevated non-specific signal. The presence of human serum in binding buffer overall reduced the non-specific binding.

### Example 11: Adiponectin ELISA

Nanoparticles were prepared and purified according to the procedure of SL-113, with a carboxylic acid-functionalized DSPE-PEG derivative, DSPE-PEG-2k-COOH, replacing the DSPE-PEG-2k-Biotin used for SL-113. After purification, the carboxylic acid was activated in MES buffer at pH 5, through the addition of 2 mM 1-Ethyl-3-(3-dimethylaminopropyl)-carbodiimide (hydrochloride salt) and 5 mM N-hydroxysulfosuccinimide. The activation reaction proceeded for 15 min at room temperature, after which unreacted EDC was deactivated with 2 mM 2-mercaptoethanol. A commercial anti-biotin antibody (Abcam) at ˜1 mg/mL in 1×PBS was then added to the activated particles, and the pH was titrated to ˜7.2. The coupling reaction proceeded for 2 hours at room temperature with stirring. The reaction volume was then loaded into a 300 kD cutoff dialysis tubing (Spectrum Labs) and dialyzed into a 1:1000 dilution of 1×PBS overnight. The antibody-functionalized nanoparticles were collected and stored at 4° C. until use.

A sandwich immunoassay was performed according to the kit instructions. A RayBiotech human adiponectin ELISA kit was purchased (ELH-ADIPONECTIN-001), and two sets of standards, each in triplicate, were prepared according to the kit instructions. In brief, to a kit-supplied capture antibody-coated 96-well microplate, varying concentrations of the provided standard were added and binding proceeded for 2 hours. The wells were washed 4 times followed by the addition of the kit-provided biotinylated detection antibody, and then diluted according to instructions. Binding proceeded for 1 hour followed by washing. The kit-provided streptavidin-HRP conjugate was then added to the first set of standards and the anti-biotin-functionalized nanoparticles were added to the second set. Binding proceeded for 30 minutes and was followed by washing. The kit-supplied, TMB-based detection solution was added to the HRP-containing wells and the reaction was allowed to proceed for 20 min, at which point the stop solution was added and the signal was measured by absorbance. In parallel, the 1:1 ethanol: 1M NaOH nanoparticle signal-generating solution was added to nanoparticle-containing wells and the signal was read fluorescently.

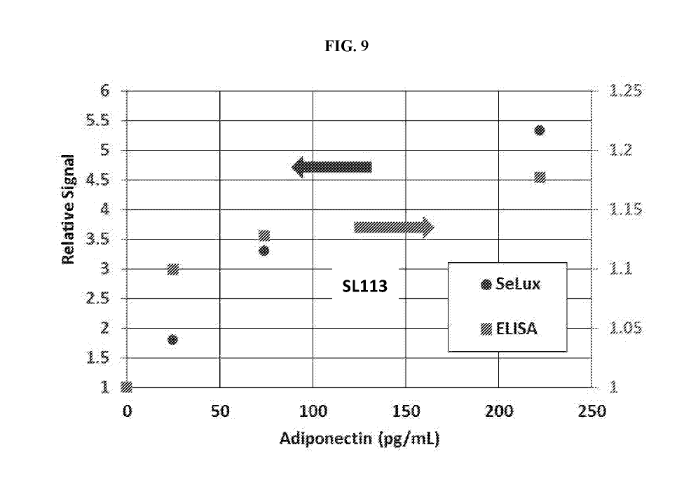

All measurements were taken with a Molecular Devices SpectraMax X2 microplate reader and the data shown were normalized to the zero-adiponectin point (FIG. 9).

### Example 12: Effect of Particle Size on Binding Efficiency

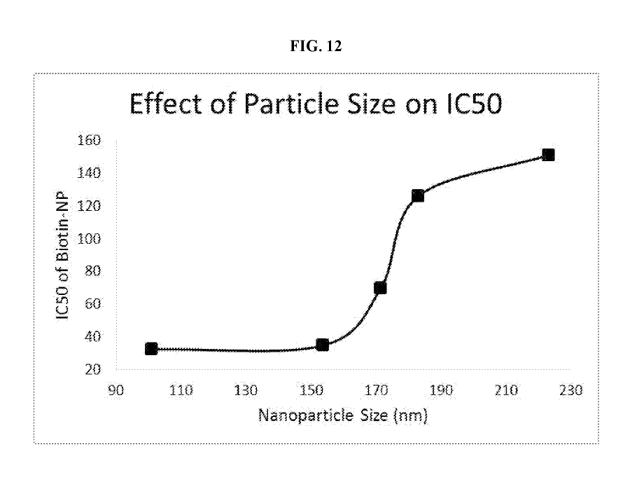

FIG. 12 illustrates the effect of nanoparticle size for nanoparticles comprising fluorescein dilaurate binding. The binding affinities of biotin coated fluorescein dilaurate nanoparticles (bt-FDL) were determined in a commercially available streptavidin coated 96 well plate (Thermofisher). The plate was washed three times with PBST prior to the addition of 100 μL bt-FDL (200 ng/mL biotin) and 100 μL biotin (final concentrations: 10⁻²-10⁵ M). After incubating for 30 minutes at 37° C. with 575 rpm shaking, the plate was washed three times with PBST, developed with (NaOH:MeOH 50:50, 15 min), and fluorescein units were measured using a plate reader (excitation: 485 nm, emission: 530 nm). The data is presented in the table below (Table 5):

TABLE 5

| FDL Amount (mg) | Diameter (nm) | IC₅₀ |
|---|---|---|
| 20 | 100.9 | 32.440 |
| 40 | 153.6 | 34.89 |
| 80 | 171.4 | 69.92 |
| 160 | 182.9 | 126.1 |
| 320 | 223.1 | 151.2 |

The plot in FIG. 12 illustrates the relationship between nanoparticle size and binding efficiency as measured by IC₅₀. IC₅₀ is defined as the concentration at which half of the competitor's binding is inhibited. Therefore the lower the IC₅₀ value, the more effective the biotinylated nanoparticle is at competing against the free biotin.

### Example 13: Effect of Nanoparticle Functional Group Concentration on Specific and Nonspecific Binding

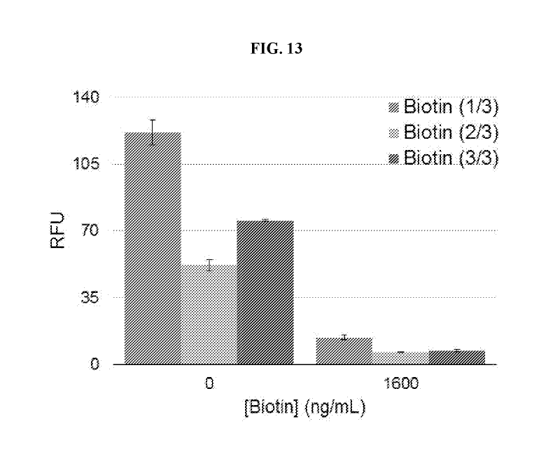

FIG. 13 illustrates the effect nanoparticle functional group concentration on specific and nonspecific binding. Three samples of nanoparticles were prepared: one with 1/3 of the surface functionalized with biotin, one with 2/3 of the surface functionalized with biotin, and one with 3/3 of the surface functionalized with biotin. The remainder of the surface was functionalized with a free amine. The table (Table 6) below lists the amount of biotin-DSPE, amine-DSPE, and FDL used to prepare each sample.

TABLE 6

| | | | |
|---|---|---|---|
| Sample | Biotin-DSPE | Amine-DSPE | FDL |

| | | | | | |
|---|---|---|---|---|---|
| Biotin (⅓) | 5.3 | mg | 10.7 | mg | 20 mg |
| Biotin (⅔) | 10.7 | mg | 5.3 | mg | 20 mg |
| Biotin ( 3/3) | 16 | mg | 0 | mg | 20 mg |

FIG. 13 is a bar graph which illustrates the specific and non-specific binding of nanoparticles (as measured by relative fluorescence units (RFU)) to concentration of biotin present in a sample. The assays were prepared using a streptavidin coated plate with varying amounts of biotin on the surface. The streptavidin plate was washed (3×, PBST) prior to incubation with 100 μL bt-FDL (200 ng/mL biotin) and 100 μL biotin (0 or 1600 ng/mL, specific or nonspecific binding, respectively). After 30 minutes at 37° C. with 575 rpm shaking, the plate was washed (3×, PBST), developed (100 μL, NaOH:MeOH 50:50, 15 min) and relative fluorescent units were measured using a plate reader (excitation: 485 nm, emission: 530 nm). The concentration of biotin on the surface of fluorescein dilaurate nanoparticles effects the overall fluorescent signal (0 ng/mL biotin) and nonspecific binding (1600 ng/mL). Nanoparticles with 1/3 biotin surface coating produced the highest relative fluorescent signal of 130 RFU but complete biotin surface coverage produced a 10.5 fold increase of the non-specific binding.

### Example 14: Percent Recovery of P4 from Samples Containing Known Concentrations Using Nanoparticles with a P4 Competitive Immunoassay

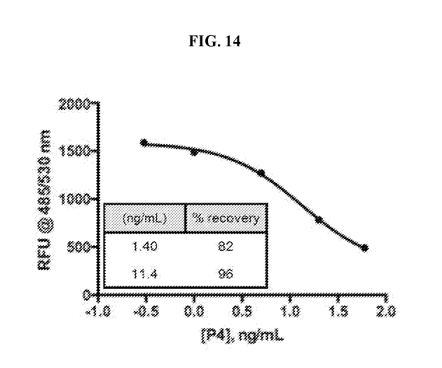

FIG. 14 is a plot illustrating the logarithmic relationship between the concentration of target sample (P4) and the presence of bound nanoparticles as measured in relative fluorescence units (RFU). A rabbit anti-progesterone coated plate was incubated with 0.05 ng/mL p4-11-botin and varying amounts of P4 (0-60 ng/mL) for 1 hour at 37° C. with shaking at 575 rpm. The plate was than washed three times and incubated with neutravidin (500 ng/mL). After 5 mins, the plate was washed again and incubated with 100 L of 50 ng/mL biotin-FDL. After 30 minutes at 37° C. with 575 rpm shaking, the plate was washed (5×, PBST), developed (100 uL, NaOH:MeOH 50:50, 15 min) and relative fluorescent units were measured using a plate reader (excitation: 485 nm, emission: 530 nm). Percent recoveries for test clinical samples were determined by dividing the value obtained for P4 concentration from an exponential best-fit curve to the 5 standards by the known concentrations of P4 in clinical samples and multiplying by 100. Acceptable ranges for the assay are 80-120% thus these samples fall within range.

### Example 15: Investigation of Nanoparticle Stability

The table below illustrates the stability of the nanoparticles synthesized by methods described herein (Table 7).

TABLE 7

| Time | | ⅓ | | ⅔ | 3/3 |
|---|---|---|---|---|---|
| (hours) | ⅓ | Cholate | ⅔ | Cholate | Cholate |
| 5 | 112 | 105.1 | 96.81 | 98.20 | 95.65 |
| PDI | 0.148 | 0.122 | 0.132 | 0.112 | 0.131 |
| 8 | 114.1 | 109.8 | 99.35 | 101.1 | 94.57 |
| PDI | 0.143 | 0.127 | 0.132 | 0.090 | 0.121 |
| 24 | 121.2 | 114.3 | 99.35 | 98.92 | 94.39 |
| PDI | 0.137 | 0.148 | 0.132 | 0.132 | 0.120 |
| 72 | 130.6 | 135.0 | 104.4 | 105.7 | 100.1 |
| PDI | 0.099 | 0.113 | 0.132 | 0.114 | 0.183 |
| 168 | 133.0 | 133.4 | 103.7 | 103.7 | 100.1 |
| PDI | 0.136 | 0.105 | 0.172 | 0.214 | 0.167 |

The nanoparticles were stored in deionized water at 37° C. for the time indicated. Five lots were prepared according to the methods described in Example 3. The fraction represents the percentage of the surface of the nanoparticle functionalized with biotin. The lots labeled “Cholate” are lots where cholate was added as a surfactant.

Instability of particles is indicated by the size and PDI of the particles increasing over time. The relatively small change in PDI despite minor particle growth illustrate that the particles are not degrading or aggregating beyond an acceptable value.

### Example 16: Sandwich Immunoassay Performed with FDL Particles Stored for 4+ Months at Room Temperature

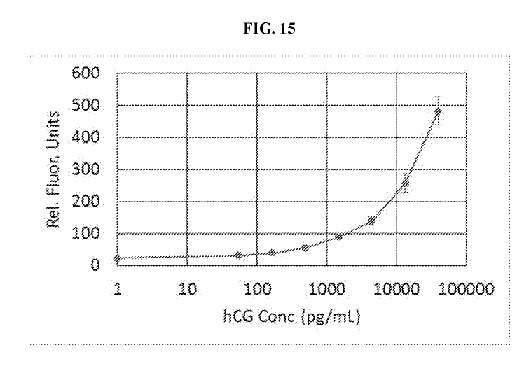

FIG. 15 is a plot of particle fluorescence strength in a sandwich immunoassay performed with nanoparticles stored for four months in deionized water. The nanoparticles were tested by using them as the reporters in a human chorionic gonadotropin (hCG) immunoassay. A human hCG ELISA microplate kit (Ray Biotech) was used and a standard curve was prepared as instructed. The neutravidin-functionalized, FDL-loaded nanoparticles were used in place of the avidin-horseradish peroxidase enzyme during the final binding step. A solution of ethanol and 1M sodium hydroxide was then added and the fluorescence at 490/530 nm was measured.

### IX. EXEMPLARY EMBODIMENTS

Exemplary, non-limiting embodiments 1 to 129 are provided herein.

### Embodiment 1

A nanoparticle comprising
(a) a transition-metal catalyst; and
(b) one or more matrix-forming agents providing a dissociable matrix, wherein the transition-metal catalyst is embedded in the matrix;
wherein
said transition-metal catalyst of (a) comprises a structure according to formula I,

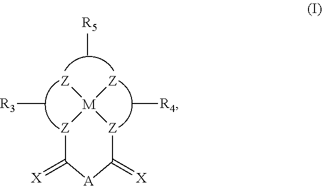

or an oxidized or reduced form thereof, wherein
M is a metal;
A is —CR₁R₂— or —NR_{1′}—;
   wherein when A is —CR₁R₂—, R₁ and R₂ are the same or different, linked or nonlinked, and each is independently selected from the group consisting of substituents which are unreactive, form strong bonds intramolecularly within said R₁ and R₂ and with the carbon C to which they are bound, are sterically hindered and are conformationally hindered such that oxidative degradation of a metal complex of the compound is restricted when the complex is in the presence of an oxidizing medium; and
   wherein when A is —NR_{1′}—, R_{1′} is C₁₋₂₀ alkyl, C₂₋₂₀ alkenyl, C₂₋₂₀ alkynyl, or phenyl;
   Z is a metal complexing atom selected from the group consisting of N and O;
   X is a functionality;
   wherein both Z and X are resistant to oxidative degradation such that each confers resistance to oxidative degradation to the metal complex of the compound when the complex is in the presence of an oxidizing medium;
   R₃ is a unit joining the adjacent Z atoms selected from the group consisting of:

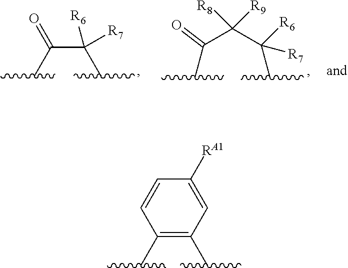

and wherein R₆, R₇, R₈ and R₉ pairwise and cumulatively are the same or different and each is selected from the group consisting of hydrogen, C₁₋₂₀ alkyl, C₂₋₂₀ alkenyl, C₂₋₂₀ alkynyl, C₆₋₁₀ aryl, and halogen; or any pair of R₆, R₇, R₈ and R₉ can, together with the atoms to which they are attached, form a C₄₋₁₀ cycloalkyl;
   R^{A1} is hydrogen, halogen, or —X¹—Y¹—Z¹, wherein
      X¹ is —C(R^{X1})₂—, —C(O)—, —C(O)O—, —C(O)NH—, —CR^{X1}═CR^{X1}—, —NR^{X1}—, —NR^{X1}C(O)—, —O—, or —OC(O)—, wherein R^{X1} is hydrogen or C₁₋₆ alkyl;
      Y¹ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y¹ are optionally and independently replaced by -Cy¹-,
   —NR^{Y1}—, —N(R^{Y1})C(O)—, —C(O)N(R^{Y1})—, —O—, —C(O)—, —OC(O)—, —C(O)O—, or —N═N—, wherein R^{Y1} is hydrogen or C₁₋₆ alkyl; and
      each Cy¹ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
      Z¹ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
R₄ is a unit joining the adjacent Z atoms comprised of

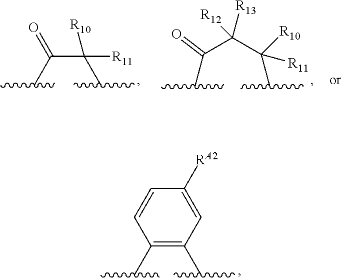

wherein R₁₀, R₁₁, R₁₂ and R₁₃ pairwise and cumulatively are the same or different and each is selected from the group consisting of hydrogen, C₁₋₂₀ alkyl, C₂₋₂₀ alkenyl, C₂₋₂₀ alkynyl, C₆₋₁₀ aryl, and halogen; or any pair of R₁₀, R₁₁, R₁₂ and R₁₃ can, together with the atoms to which they are attached, form a C₄₋₁₀ cycloalkyl;
   R^{A2} is hydrogen, halogen, or —X²—Y²—Z², wherein
      X² is —C(R^{X2})₂—, —C(O)—, —C(O)O—, —C(O)NH—, —CR^{X2}═CR^{X2}—, —NR^{X2}—, —NR^{X2}C(O)—, —O—, or —OC(O)—, wherein R^{X2} is hydrogen or C₁₋₆ alkyl;
      Y² is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y² are optionally and independently replaced by -Cy²-,
   —NR^{Y2}—, —N(R^{Y2})C(O)—, —C(O)N(R^{Y2})—, —O—, —C(O)—, —OC(O)—, —C(O)O—, or —N═N—, wherein R^{Y2} is hydrogen or C₁₋₆ alkyl; and
      each Cy² is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
      Z² is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl
R₅ is a unit joining adjacent Z atoms selected from the group consisting of
   (i)

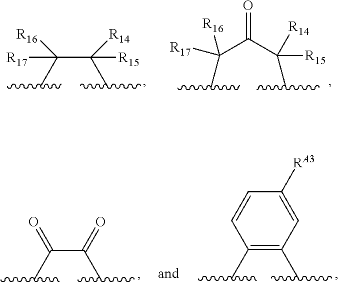

wherein R₁₄, R₁₅, R₁₆ and R₁₇ are the same or different and each is hydrogen, C₁₋₂₀ alkyl, C₂₋₂₀ alkenyl, C₂₋₂₀ alkynyl, C₆₋₁₀ aryl, and halogen;
or any pair of R₁₄, R₁₅, R₁₆ and R₁₇ can, together with the atoms to which they are attached, form a C₄₋₁₀ cycloalkyl;
R^{A3} is hydrogen, halogen, or —X³—Y³—Z³, wherein
   X³ is —C(R^{X3})₂—, —C(O)—, —C(O)O—, —C(O)NH—, —CR^{X3}═CR^{X3}—, —NR^{X3}—, —NR^{X3}C(O)—, —O—, or —OC(O)—, wherein R^{X3} is hydrogen or C₁₋₆ alkyl;
   Y³ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y³ are optionally and independently replaced by -Cy³-,
—NR^{Y3}—, —N(R^{Y3})C(O)—, —C(O)N(R^{Y3})—, —O—, —C(O)—, —OC(O)—, —C(O)O—, or —N═N—, wherein R^{Y3} is hydrogen or C₁₋₆ alkyl; and
   each Cy³ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
   Z³ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and optionally a counter ion selected from H₂O, ammonium, and halogen.

### Embodiment 2

The nanoparticle of embodiment 1, wherein each Z is N.

### Embodiment 3

The nanoparticle of embodiment 1, wherein each of R₁ and R₂ is selected, independently, from the group consisting of hydrogen, halogen, and C₁₋₂₀ alkyl, or wherein R₁ and R₂ link to form a C₃₋₁₀ cycloaliphatic group.

### Embodiment 4

The nanoparticle of embodiment 1, wherein R₃ is a unit joining the adjacent Z atoms comprised of

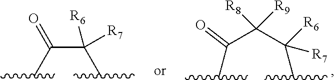

wherein each of R₆, R₇, R₈ and R₉ is, independently halogen, C₁₋₂₀ alkyl, C₂₋₂₀ alkenyl, or C₂₋₂₀ alkynyl; or
R₆ and R₇, or R₈ and R₉, link to form a C₃₋₁₀ cycloaliphatic group.

### Embodiment 5

The nanoparticle of embodiment 1, wherein R₄ is a unit joining the adjacent Z atoms comprised of

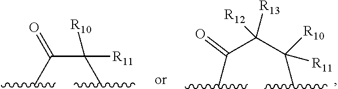

wherein each of R₁₀, R₁₁, R₁₂ and R₁₃ is, independently, halogen, C₁₋₂₀ alkyl, C₂₋₂₀ alkenyl, or C₂₋₂₀ alkynyl; or
R₁₀ and R₁₁, or R₁₂ and R₁₃, link to form a C₃₋₁₀ cycloaliphatic group.

### Embodiment 6

The nanoparticle of embodiment 1, wherein R₅ is a unit joining adjacent Z atoms selected from the group consisting of

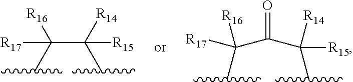

wherein each of R₁₄, R₁₅, R₁₆ and R₁₇ is independently selected from C₁₋₂₀ alkyl, C₆₋₁₂ aryl, and halogen, or R₁₄ and R₁₅, or R₁₆ and R₁₇, link to form a C₃₋₁₀ cycloaliphatic group.

### Embodiment 7

The nanoparticle of embodiment 1, wherein R₅ is an optionally-substituted aryl or heteroaryl group.

### Embodiment 8

The nanoparticle of embodiment 1, wherein one of any one of R₃, R₄, and R₅ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 9

The nanoparticle of any one of embodiments 1 to 8, wherein A is —CR₁R₂.

### Embodiment 10

The nanoparticle of any one of embodiments 1 to 8, wherein A is —NR_{1′}—.

### Embodiment 11

The nanoparticle of any one of embodiments 1 to 10, wherein the embedding of the catalyst in the matrix is not primarily governed by electrostatic interactions;

### Embodiment 12

A nanoparticle comprising
(a) a transition-metal catalyst; and
(b) one or more matrix-forming agents providing a dissociable matrix, wherein the transition-metal catalyst is embedded in the matrix;
wherein
said transition-metal catalyst of (a) comprises a structure according to formula II,

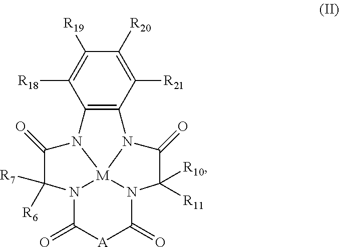

or an oxidized or reduced form thereof, wherein
   M is a metal;
A is —CR₁R₂— or —NR_{1′}—;
   wherein when A is —CR₁R₂—, each of R₁ and R₂ is, independently, hydrogen, C₁₋₂₀ alkyl, C₂₋₂₀ alkenyl, C₂₋₂₀ alkynyl, C₆₋₁₄ aryl, or halogen, or R₁ and R₂ may form, together with the carbon atom to which both are bound, a 3-6 membered ring; and
   wherein when A is —NR_{1′}—, R_{1′} is C₁₋₂₀ alkyl, C₂₋₂₀ alkenyl, C₂₋₂₀ alkynyl, or phenyl;
   each of R₆, R₇, R₁₀, and R₁₁ is, independently, hydrogen, C₁₋₂₀ alkyl, C₂₋₂₀ alkenyl, C₂₋₂₀ alkynyl, C₆₋₁₄ aryl, or halogen, or R₁ and R₂, or R₃ and R₄, or R₅ and R₆ may form, together with the carbon atom to which both are bound, a 3-10 membered ring; and
   each of is R₁₈, R₁₉, R₂₀, and R₂₁ is, independently, halogen, hydrogen, C₁₋₂₀ alkyl, C₂₋₂₀ alkenyl, C₂₋₂₀ alkynyl, C₆₋₁₄ aryl, amino, nitro, azido, cyano, —OH, C₁₋₂₀ alkoxy, —SH, C₁₋₂₀ thioalkoxy, C₆₋₁₄ aryloxy, —CO₂H, a carboxylic ester, an N-hydrosuccinimide ester group, an isothiocyanate group, an isocyanide group, or a 5-10-membered heterocyclic group.

### Embodiment 13

The nanoparticle of embodiment 12, wherein said transition-metal catalyst has a structure according to formula IIA,

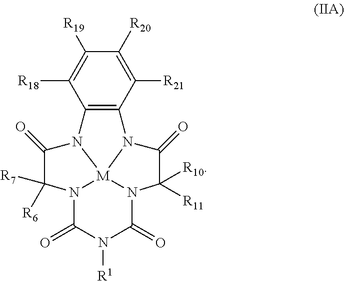

### Embodiment 14

The nanoparticle of embodiment 12, wherein said transition-metal catalyst has a structure according to formula IIB,

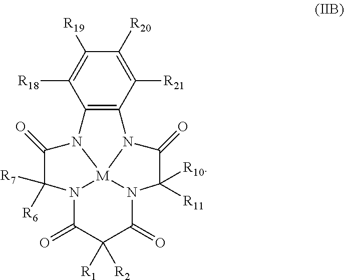

### Embodiment 15

The nanoparticle of any one of embodiments 12-14, wherein each of R₁ and R₂ is selected, independently, from the group consisting of hydrogen, halogen, and C₁₋₂₀ alkyl, or wherein R₁ and R₂ link to form a C₃₋₁₀ cycloaliphatic group.

### Embodiment 16

The nanoparticle of any one of embodiments 12-15, wherein one or more of R₆, R₇, R₁₀, and R₁₁ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 17

The nanoparticle of any one of embodiments 12-15, wherein one or more of R₁₈, R₁₉, R₂₀, and R₂₁ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 18

The nanoparticle of embodiment 12, comprising a transition metal catalyst having a structure that is

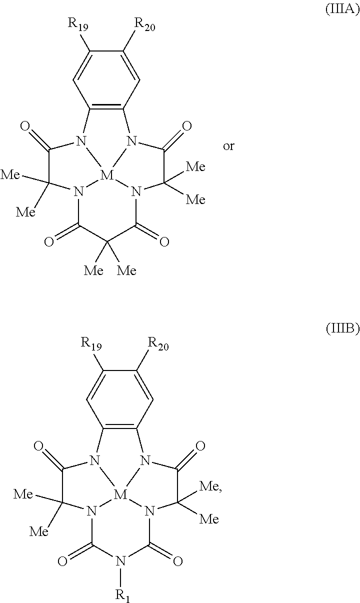

or an oxidized or reduced form thereof.

### Embodiment 19

The nanoparticle of embodiment 18, wherein one or both of R₁₉ and R₂₀ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 20

The nanoparticle of embodiment 18, wherein the transition metal catalyst has a structure according to formula (IIIB) and R¹ optionally comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 21

The nanoparticle of embodiment 12, comprising a transition-metal catalyst having a structure that is,

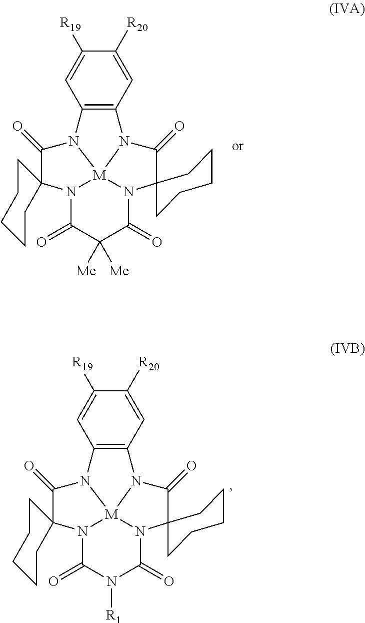

or an oxidized or reduced form thereof.

### Embodiment 22

The nanoparticle of embodiment 21, wherein one or both of R₁₉ and R₂₀ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 23

The nanoparticle of embodiment 22, wherein one or both of R₁₉ and R₂₀ is a norbornene or cyclooctene.

### Embodiment 24

The nanoparticle of embodiment 21, wherein the transition metal catalyst has a structure according to formula (IVB) and R¹ optionally comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 25

The nanoparticle of any one of embodiments 1-24, wherein M is a group 6, 7, 8, 9, 10, or 11 metal.

### Embodiment 26

The nanoparticle of embodiment 25, wherein M is Cr, Mn, Fe, Co, Ni, or Cu.

### Embodiment 27

The nanoparticle of any one of embodiments 12 to 26, wherein the embedding of the catalyst in the matrix is not primarily governed by electrostatic interactions;

### Embodiment 28

A nanoparticle comprising
(a) a transition-metal catalyst; and
(b) one or more matrix-forming agents providing a dissociable matrix, wherein the transition-metal catalyst is embedded in the matrix;
wherein
   said transition-metal catalyst of (a) comprises a structure according to formula V,

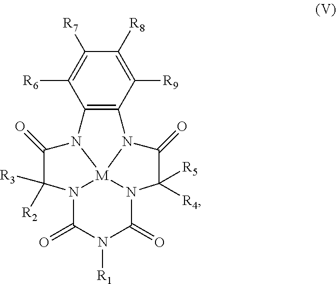

or an oxidized or reduced form thereof, wherein
   M is a metal selected from the group consisting of Cr, Mn, Fe, Cu, Ni and Co;
   R₁ is C₁₋₂₀ alkyl, C₂₋₂₀ alkenyl, C₂₋₂₀ alkynyl, or phenyl;
   each of R₂, R₃, R₄, and R₅ is, independently, hydrogen, C₁₋₂₀ alkyl, C₂₋₂₀ alkenyl, C₂₋₂₀ alkynyl, or phenyl, or R₂ and R₃, or R₄ and R₅, combine to form a C₃₋₁₀ cycloaliphatic;
   each of R₆, R₇, R₈, and R₉ is, independently, amino, nitro, azido, cyano, hydrogen, halogen, —NO₂, —COOH, —COOR₁₀, —COCl, —CN, C₁₋₂₀ alkyl, C₂₋₂₀ alkenyl, or C₂₋₂₀ alkynyl, wherein at least one of R₆, R₇, R₈, and R₉ is halogen, —NO₂, —COOH, —COOR₁₀, —COCl, or —CN;
   R₁₀ is C₁₋₂₀ alkyl, C₂₋₂₀ alkenyl, C₂₋₂₀ alkynyl, phenyl, or 5-to-10-membered heterocyclyl.

### Embodiment 29

The nanoparticle of embodiment 28, wherein one or both of R₇ and R₈ is halogen, —NO₂, —COOH, —COOR₁₀, —COCl, —CN, or a N-hydroxysuccinimide ester group.

### Embodiment 30

The nanoparticle of embodiment 28, wherein R₁, R₇, and/or R₈ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 31

The nanoparticle of embodiment 28, wherein one or more of R₂, R₃, R₄, and R₅ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

### Embodiment 32

The nanoparticle of embodiment 28, wherein each of R₂, R₃, R₄, and R₅ is C₁ alkyl.

### Embodiment 33

The nanoparticle of any one of embodiments 1-32, wherein M is Fe(III) and the transition-metal catalyst further comprises a cation having a charge of +1.

### Embodiment 34

The nanoparticle of any one of embodiments 1-33, wherein the transition-metal catalyst can mediate an oxidative or reductive transformation on a compound.

### Embodiment 35

The nanoparticle of embodiment 34, wherein the transition-metal catalyst can mediate an oxidative reaction on a compound.

### Embodiment 36

The nanoparticle of embodiment 35, wherein said compound is selected from: hydroethidine (HE); 1,3-diphenylisobenzofuran (DPBF), 2-(2-pyridyl)-benzothioazoline; 2,7-dichlorodihydrofluorescein (DCFH); 7-hydroxy-6-methoxy coumarin (scopoletin); N-acetyl-3,7-dihydroxyphenoxazine (Amplex Red); 4-hydroxy-3-methoxy-phenylacetic acid (HVA or homovanillic acid); dihydrorhodamine 123 (DHR); 4-(9-anthroyloxy)-2,2,6,6,-tetramethylpiperidine-1-oxyl; 1,3-cyclohexanedione (CHD); sodium terephthalate; coumarin-3-carboxylic acid (3-CCA); N-succinimidyl ester of coumarin-3-carboxylic acid (SECCA); 2-[6-(4′-hydroxy)phenoxy-3H-xanthen-3-on-9-yl]benzoic acid (HPF); 2-[6-(4′-amino)phenoxy-3H-xanthen-3-on-9-yl]benzoic acid (APF); cis-parinaric acid (cis-PnA, (18:14):9,11,13,15-cis-trans-trans-cis-octadecaenoic acid); 4,4-difluoro-5-(4-phenyl-1,3-butadienyl)-4-bora-3a,4a-diaza-s-indacene-3-undecanoic acid (C₁₁-BODIPY); lipophilic fluorescein derivatives; dipyridamole; diphenyl-1-pyrenylphosphine (DPPP); 2,7-dichlorodihydrofluorescein acetate (DCFH-DA); beta-physcoerythrin; fluorescein; and 6-carboxyfluorescein, or a derivative thereof.

### Embodiment 37

A nanoparticle comprising
(a) a transition-metal catalyst having the structure M(X)ₙ(R)ₒ, wherein
   M is a transition-metal;
   n is 0, 1, 2, 3, or 4;
   o is 2, 3, 4, 5, or 6;
   X is an ion of a Group V, VI, or VII element;
   R is a ligand selected from monodentate phosphine ligands, bidentate phosphine ligands, monodentate Schiff base ligands, bidentate Schiff base ligands, tridentate Schiff base ligands, macrocyclic ligands, pentamethylcyclopentadiene, monodentate arsine, or N-heterocyclic carbene ligands; and
(b) one or more matrix-forming agents providing a dissociable matrix, wherein the transition-metal catalyst is embedded in the matrix;
wherein
said transition-metal catalyst of (a) can catalyze a bond formation reaction or a bond cleavage reaction that modulates the fluorescent or chromogenic properties of a substrate compound.

### Embodiment 38

The nanoparticle of embodiment 37, wherein the embedding of the catalyst in the matrix is not primarily governed by electrostatic interactions.

### Embodiment 39

The nanoparticle of embodiment 37 or 38, wherein the matrix sequesters the transition-metal catalyst until said matrix is dissociated.

### Embodiment 40

The nanoparticle of any one of embodiments 37-39 wherein said substrate compound comprises a functional group that quenches fluorescence when covalently bound to the substrate compound.

### Embodiment 41

The nanoparticle of any one of embodiments 37-40, wherein said transition-metal catalyst can induce fluorescence by mediating a bond cleavage reaction of the fluorescence quenching functional group in the substrate compound.

### Embodiment 42

The nanoparticle of embodiment 41, wherein said substrate compound is a halogenated boron dipyrromethane (BODIPY) compound.

### Embodiment 43

The nanoparticle of embodiment 42, wherein said substrate compound has a structure that is

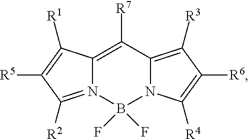

each of R¹, R², R³, R⁴, R⁵, and R⁶ is, independently, H, halogen, CN, C₁₋₂₀ alkyl, C₂₋₂₀ alkenyl, C₂₋₂₀ alkyl, C₁₋₂₀ alkoxy, —O(CH₂CH₂O)ₙCH₃, or —OCH((CH₂CH₂O)ₙCH₃)₂,
R⁷ is H, halogen, CN, C₁₋₂₀ alkyl, C₂₋₂₀ alkenyl, C₂₋₂₀ alkyl, C₁₋₂₀ alkoxy, C₆₋₁₀ aryl, or 5-to-10-membered heteroaryl;
each n is, independently, an integer from 1 to 6; and
wherein at least one of R¹, R², R³, R⁴, R⁵, R⁶, and R⁷ is halogen.

### Embodiment 44

The nanoparticle of embodiment 43, wherein each of R¹, R², R³, and R⁴ is, independently, C₁₋₂₀ alkyl.

### Embodiment 45

The nanoparticle of embodiment 43 or 44, wherein at least one of R¹, R², R³, and R⁴ comprises a carboxylic acid substituent.

### Embodiment 46

The nanoparticle of any one of embodiments 43-45, wherein one or both of R⁵ and R⁶ is halogen.

### Embodiment 47

The nanoparticle of 46, wherein one or both of R⁵ and R⁶ is bromo or iodo.

### Embodiment 48

The nanoparticle of embodiment 43, wherein the substrate compound has the following structure,

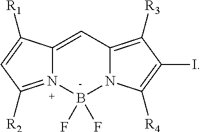

### Embodiment 49

The nanoparticle of any one of embodiments 43-47, wherein R⁷ is H or phenyl.

### Embodiment 50

The nanoparticle of embodiment 37, wherein M is Pd(0), Pd(II), Rh(I), Rh(III), Ir(I), Ir(III), Ru(II), Ru(III), Pt(0), Pt(II), or Cu(II).

### Embodiment 51

The nanoparticle of embodiment 50, wherein said transition-metal catalyst comprises monodentate phosphine ligands, bidentate phosphine ligands, monodentate Schiff base ligands, bidentate Schiff base ligands, tridentate Schiff base ligands, macrocyclic ligands, pentamethylcyclopentadiene, monodentate arsine, or N-heterocyclic carbene ligands.

### Embodiment 52

The nanoparticle of embodiment 51, wherein said transition-metal catalyst comprises a ligand selected from: triarylphosphines, trialkylphosphines, aryldialkylphosphines, 1,1′-bis(diphenylphosphino)ferrocene, tricycloalkylphosphine, (1,1′-biphenyl-2-yl)dicyclohexylphosphine, aryldicycloalkylphosphines, 2,6-bis[l-(phenyl)iminoethyl]pyridine, 3-[[3-[(E)-[[2,6-bis(1-methylethyl)phenyl]imino]methyl]-4-hydroxyphenyl]methyl]-1-methyl-imidazolium chloride, 3,7,11,17-tetraazabicyclo[11.3.1]heptadeca-1(17),13,15-triene, tetrasulfophthalocyanine, pentamethylcyclopentadiene, triarylarsines, 1,3-diisopropylimidazolium tetrafluoroborate, 1,3-bis(1-adamantanyl)imidazolium tetrafluoroborate, 1,3-bis-(tert-butyl)-4,5-dihydro-1H-imidazolium tetrafluoroborate, N,N′-(2,4,6-trimethyl)dihydroimidazolium chloride, and N,N′-(2,6-diisopropylphenyl)dihydroimidazolium chloride.

### Embodiment 53

The nanoparticle of embodiment 50, wherein M is Pd(II) or Pd(0).

### Embodiment 54

The nanoparticle of embodiment 50, wherein the nanoparticle comprises Pd(PCy₃)₂Cl₂, Pd(PPh₃)₂Cl₂, Pd(PPh₃)₄, Pd₂(dba)₃, Pd(TFA)₂, Pd(MeCN)₂Cl₂, Pd(acac)₂, Pd(amphos)Cl₂, Pd(dppf)Cl₂, Pd(dtbpf)Cl₂, Na₂PdCl₄, PdC, (NH₄)₂PdCl₄, PdBr₂, Pd(OAc)₂, or tris(dibenzylideneacetone)dipalladium(0).

### Embodiment 55

The nanoparticle of any one of embodiments 1-54, wherein at least one of said one or more matrix-forming agents comprises organic polymers, waxes, fats, oils, surfactants, or a combination thereof.

### Embodiment 56

The nanoparticle of embodiment 55, wherein said at least one matrix-forming agent comprises an organic polymer.

### Embodiment 57

The nanoparticle of embodiment 56, wherein said organic polymer comprises a hydrolyzable constituent.

### Embodiment 58

The nanoparticle of any one of embodiments 1-54, wherein at least one of said one or more matrix-forming agent forms an inorganic matrix.

### Embodiment 59

The nanoparticle of any one of embodiments 1-58, wherein the matrix comprises a covalent bond to the transition-metal catalyst.

### Embodiment 60

The nanoparticle of any one of embodiments 1-59, wherein the matrix comprises a non-covalent interaction with the transition-metal catalyst.

### Embodiment 61

The nanoparticle of embodiment 60, wherein the non-covalent interaction with the transition-metal catalyst is a hydrophobic interaction, a hydrogen bonding interaction, or a van der Waals interaction.

### Embodiment 62

The nanoparticle of any one of embodiments 1-61, wherein the nanoparticle comprises an outer surface that comprises one or more functional groups for conjugating the nanoparticle to a binding agent.

### Embodiment 63

The nanoparticle of embodiment 62, wherein the nanoparticle further comprises an inner layer between the matrix core and the outer surface.

### Embodiment 64

The nanoparticle of embodiment 62 or 63, wherein the binding agent comprises an antibody, ligand, protein, small molecule, aptamer, ss-DNA, ss-RNA, or ss-PNA.

### Embodiment 65

The nanoparticle of any one of embodiments 1-64, wherein the matrix comprises one or more catalyst species.

### Embodiment 66

The nanoparticle of any one of embodiments 1-65, wherein the matrix further comprises a compound that is a chemiluminophore, a chemiluminophore precursor, an absorber, or an absorber precursor.

### Embodiment 67

The nanoparticle of embodiment 66, wherein the matrix comprises solvent dyes and/or water-soluble dyes.

### Embodiment 68

The nanoparticle of embodiment 66, wherein the matrix comprises fluorescein dilaurate, fluorescein, rhodamine, rhodamine B octadecyl ester, Oregon green, eosin, Texas red, BODIPY, AlexaFluor, Atto, cyanine, indocarbocyanine, oxacarbocyanine, thiacarbocyanine, merocyanine, dansyl, prodan, coumarin, pyridyloxazole, nitrobenzoxadiazole, benzoxadiazole, anthraquinone, cascade blue, Nile red, Nile blue, cresyl violet, proflavin, acridine orange, acridine yellow, auramine, crystal violet, malachite green, porphin, phthalocyanine, bilirubin, 9,10-diphenylanthracene, 1-chloro-9,10-diphenylanthracene, 9,10-bis(phenylethynyl)anthracene, 1-chloro-9,10-bis(phenylethynyl)anthracene, 2-chloro-9,10-bis(phenylethynyl)anthracene, 1,8-dichloro-9,10-bis(phenylethynyl)anthracene, rubrene, 2,4-di-tert-butylphenyl-1,4,5,8-tetracarboxynaphthalene diamie, 5,12-bis(phenylethynyl)naphthacene, violanthrone, 16,16-(1,2-ethylenedioxy)violanthrone, 16,17-dihexyloxyviolanthrone, 16,17-butyloxyviolanthrone, N,N′-bis(2,5-di-tert-butylphenyl)-3,4,9,10-perylenedicarboximide, 1-N,N′-dibutylaminoanthracene, 6-methylacridinium iodide, or luminol, or a derivative thereof.

### Embodiment 69

The nanoparticle of any one of embodiments 66-68, comprising a molar ratio of the compound: transition-metal catalyst that is about 10:1 to about 1:1, about 10:1 to about 3:1, about 8:1 to about 3:1, or about 5:1 to about 3:1.

### Embodiment 70

The nanoparticle of any one of embodiments 1-69, wherein the matrix further comprises a second transition-metal catalyst.

### Embodiment 71

A composition comprising the nanoparticles of any one of embodiments 1-70, wherein said composition has a size distribution of said nanoparticles from about 10 nm to less than 10 μm, about 10 nm to about 1 m, about 10 nm to about 1 μm, about 10 nm to about 500 nm, about 10 nm to about 300 nm, or about 50 nm to about 300 nm.

### Embodiment 72

The composition of embodiment 71, wherein said composition has a size distribution of said nanoparticles from about 25 nm to about 250 nm, about 25 nm to about 200 nm, about 25 nm to about 175 nm, about 25 nm to about 100 nm, or about 50 nm to about 100 nm.

### Embodiment 73

The composition of embodiment 72 or 73, wherein said composition has a polydispersity index of about 0.35 or below.

### Embodiment 74

The composition of embodiment 74, wherein said composition has a polydispersity index of about 0.25 or below.

### Embodiment 75

The composition of embodiment 73, wherein said composition has a polydispersity index of about 0.15 or below.

### Embodiment 76

A nanoparticle comprising
(a) a transition-metal catalyst; and
(b) one or more matrix-forming agents providing a dissociable matrix, wherein the transition-metal catalyst is embedded in the matrix;
wherein
   said transition-metal catalyst of (a) is selected from:

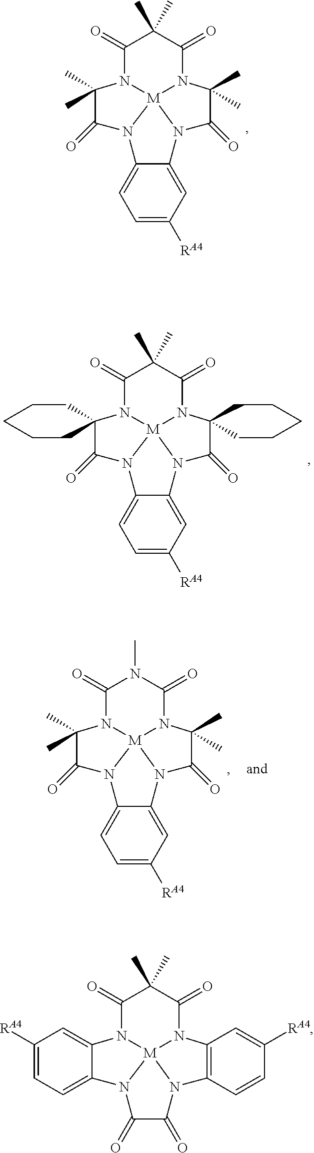

wherein
M is a metal selected from Fe, Mg, Cu, Mn, Pd, Pt, Ag, Ru, and Ce; and
   R^{A4} is hydrogen, halogen, or —X⁴—Y⁴—Z⁴, wherein
      X⁴ is —C(R^{X4})₂—, —C(O)—, —C(O)O—, —C(O)NH—, —CR^{X4}═CR^{X4} NR^{X4}—, —NR^{X4}C(O)—, —O—, or —OC(O)—, wherein R^{X4} is hydrogen or C₁₋₆ alkyl;
      Y⁴ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y⁴ are optionally and independently replaced by -Cy⁴-,
   —NR^{Y4}—, —N(R^{Y4})C(O)—, —C(O)N(R^{Y4})—, —O—, —C(O)—, —OC(O)—, —C(O)O—, or —N═N—, wherein R^{Y4} is hydrogen or C₁₋₆ alkyl; and
      each Cy⁴ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
Z⁴ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
a suitable counter ion selected from H₂O and halogen.

### Embodiment 77

A method for forming the nanoparticle of any one of embodiments 1-76 comprising
a. providing a first emulsion comprising an agent of interest, a polymeric matrix, a primary surfactant, and a first solvent system;
b. combining the first emulsion with a second solvent system to create a second emulsion;
c. mixing the second emulsion with a third solvent system to create a nanoparticle suspension; and
d. forming the water-dispersible polymeric nanoparticle in the presence of at least one secondary surfactant.

### Embodiment 78

The nanoparticle of any one of embodiments 1-76, wherein the transition metal catalyst is a signal inducing agent.

### Embodiment 79

The nanoparticle of embodiment 78, wherein the signal inducing agent reacts physically or chemically to produce a detectable signal.

### Embodiment 80

The nanoparticle of embodiment 79, wherein the detectable signal is an electrical signal.

### Embodiment 81

The nanoparticle of embodiment 79, wherein the detectable signal is a fluorescent signal.

### Embodiment 82

A liposome comprising a signal-inducing agent that is a transition metal catalyst having a structure according to formula II, formula IIA, formula IIB, formula IIIA, formula IIIB, formula IVA, formula IVB, formula V, or an oxidized or reduced form thereof.

### Embodiment 83

A liposome comprising a signal-inducing agent that is a transition metal catalyst having a structure selected from

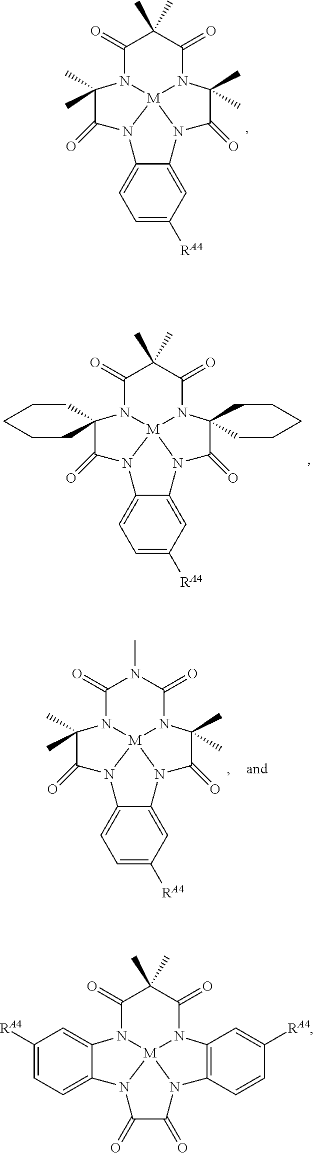

wherein
M is a metal selected from Fe, Mg, Cu, Mn, Pd, Pt, Ag, Ru, and Ce; and
   R^{A4} is hydrogen, halogen, or —X⁴—Y⁴—Z⁴, wherein
      X⁴ is —C(R^{X4})₂—, —C(O)—, —C(O)O—, —C(O)NH—, —CR^{X4}═CR^{X4}, —NR^{X4}—, —NR^{X4}C(O)—, —O—, or —OC(O)—, wherein R^{X4} is hydrogen or C₁₋₆ alkyl;
      Y⁴ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y⁴ are optionally and independently replaced by -Cy⁴-,
   —NR^{Y4}—, —N(R^{Y4})C(O)—, —C(O)N(R^{Y4})—, —O—, —C(O)—, —OC(O)—, —C(O)O—, or —N═N—, wherein R^{Y4} is hydrogen or C₁₋₆ alkyl; and
      each Cy⁴ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
Z⁴ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
   a suitable counter ion selected from H₂O and halogen.

### Embodiment 84

The liposome of embodiment 83, wherein R^{A4} is hydrogen.

### Embodiment 85

The liposome of any one of embodiments 82 to 84, wherein said transition metal is selected from the group consisting of Cr, Mn, Fe, Cu, Ni and Co.

### Embodiment 86

The liposome of embodiment 85, wherein said transition metal is Fe.

### Embodiment 87

The liposome of any one of embodiments 82 to 86 comprising an outer surface that comprises one or more functional groups for conjugating the nanoparticle to a binding agent.

### Embodiment 88

A nanoparticle comprising a polymeric matrix, wherein said polymeric matrix comprises a polymer that comprises a repeating unit comprising one or more signal-inducing agents.

### Embodiment 89

The nanoparticle of embodiment 88, wherein said signal-inducing agent is releasable.

### Embodiment 90

The nanoparticle of embodiment 89, wherein said polymer comprises a cleavable group that is within the backbone of the polymer.

### Embodiment 91

The nanoparticle of embodiment 88 or 90, wherein said polymer comprises a cleavable group that is pendant to the backbone of the polymer.

### Embodiment 92

The nanoparticle of any one of embodiments 88 to 91, wherein said polymer comprises a non-payload element for stability.

### Embodiment 93

The nanoparticle of any one of embodiments 88 to 92, wherein said polymer is covalently attached to one or more detection species.

### Embodiment 94

The nanoparticle of embodiment 88, wherein said polymer has a structure according to the following formula,

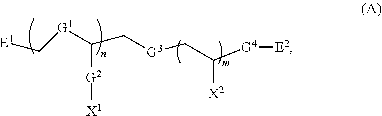

E¹ is independently hydrogen;
E² is independently hydrogen or a detection species;
each of G¹, G², G³, and G⁴ is independently a covalent bond or cleavable group;
n is independently an integer of 1 to 100;
m is independently an integer of 0 to 100;
X¹ is a signal-inducing agent; and
X² is hydrogen or non-payload element for stability.

### Embodiment 95

The nanoparticle of embodiment 94, wherein each of G¹, G², G³, and G⁴ is independently a covalent bond.

### Embodiment 96

The nanoparticle of embodiment 94, wherein one or more of G¹, G², G³, and G⁴ is independently a cleavable group.

### Embodiment 97

The nanoparticle of any one of embodiments 94-96, wherein X¹ is a signal-inducing agent comprising a transition metal catalyst.

### Embodiment 98

The nanoparticle of any one of embodiments 94-97, wherein E² is a detection species.

### Embodiment 99

The nanoparticle of embodiment 94, wherein said polymer comprises a repeating unit having a structure according to substructure S3.13,

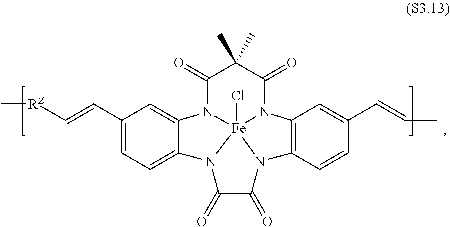

wherein R^{Z} is hydrogen, halogen, or —X^{Z1}—Y^{Z1}—Z^{Z1}, wherein X^{Z1} is —C(R^{XZ1})₂—, —C(O)—, —C(O)O—, —C(O)NH—, —CR^{XZ1}═CR^{XZ1}, —NR^{XZ1}—, —NR^{XZ1}C(O)—, —O—, or —OC(O)—, wherein R^{XZ1} is hydrogen or C₁₋₆ alkyl; Y^{Z1} is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y^{Z1} are optionally and independently replaced by -Cy^{Z11}-, —NR^{YZ1}—, —N(R^{YZ1})C(O)—, —C(O)N(R^{YZ1})—, —O—, —C(O)—, —OC(O)—, —C(O)O—, or —N═N—, wherein R^{YZ1} is hydrogen or C₁₋₆ alkyl; and each Cy^{Z1} is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and Z^{Z1} is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl.

### Embodiment 100

A nanoparticle comprising
(a) a compound that is chemiluminophore, a chemiluminophore precursor, a soluble absorber, or a soluble absorber precursor; and
(b) optionally one or more matrix-forming agents providing a matrix, wherein the compound of (a) is embedded in the matrix;
wherein
the embedding is not primarily governed by electrostatic interactions and/or the embedding is primarily governed by surfactant stabilization during formation of the matrix,
the matrix sequesters the compound of (a) until said matrix is dissociated, and
the nanoparticle comprises at least about 20 mol % of the compound of (a).

### Embodiment 101

The nanoparticle of embodiment 100, wherein the matrix sequesters the compound of (a) until said matrix is dissociated.

### Embodiment 102

The nanoparticle of embodiment 100 or embodiment 101, wherein the compound of (a) is an acylated fluorescein or an acylated rhodamine.

### Embodiment 103

The nanoparticle of embodiment 100, wherein the compound of (a) is fluorescein dilaurate, rhodamine B octadecyl ester, rhodamine B hexyl ester, Oregon green, eosin, Texas red, BODIPY, AlexaFluor, Atto, cyanine, indocarbocyanine, oxacarbocyanine, thiacarbocyanine, merocyanine, dansyl, prodan, coumarin, 3-(2-benzothiazolyl)-7-(diethylamino)coumarin (“coumarin 6”), 3-(2-N-methylbenzimidazolyl)-7-N,N-diethylaminocoumarin (“coumarin 30”), 7-amino-4-(trifluoromethyl)coumarin (“coumarin 151”), pyridyloxazole, nitrobenzoxadiazole, benzoxadiazole, anthraquinone, cascade blue, Nile red, Nile blue, cresyl violet, proflavin, acridine orange, acridine yellow, auramine, crystal violet, malachite green, porphin, phthalocyanine, bilirubin, 9,10-diphenylanthracene, 1-chloro-9,10-diphenylanthracene, 9,10-bis(phenylethynyl)anthracene, 1-chloro-9,10-bis(phenylethynyl)anthracene, 2-chloro-9,10-bis(phenylethynyl)anthracene, 1,8-dichloro-9,10-bis(phenylethynyl)anthracene, rubrene, 2,4-di-tert-butylphenyl-1,4,5,8-tetracarboxynaphthalene diamie, 5,12-bis(phenylethynyl)naphthacene, violanthrone, 16,16-(1,2-ethylenedioxy)violanthrone, 16,17-dihexyloxyviolanthrone, 16,17-butyloxyviolanthrone, N,N′-bis(2,5-di-tert-butylphenyl)-3,4,9,10-perylenedicarboximide, 1-N,N′-dibutylaminoanthracene, 6-methylacridinium iodide, or luminol, or a derivative thereof.

### Embodiment 104

The nanoparticle of any one of embodiments 100 to 103, comprising a matrix-forming agent selected from organic polymers, waxes, fats, oils, and surfactants, or a combination thereof.

### Embodiment 105

The nanoparticle of any one of embodiments 100 to 104, further comprising a transition-metal catalyst.

### Embodiment 106

A composition comprising the nanoparticles of any one of embodiments 100 to 105, wherein said composition has a size distribution of nanoparticles between about 10 nm and less than about 10 μm, between about 10 nm to about 1 μm, about 10 nm to about 1 μm, about 10 nm to about 500 nm, about 10 nm to about 300 nm, or about 50 nm to about 300 nm.

### Embodiment 107

A nanoparticle comprising
(a) a luminophore, a luminophore precursor, chemiluminophore, a chemiluminophore precursor, a soluble absorber, or a soluble absorber precursor;
(b) one or more surfactants; and
(c) a polymeric matrix-forming agent comprising a functional group,
wherein the polymeric matrix-forming agent forms a polymeric matrix; and
wherein the compound of (a) is embedded in the matrix.

### Embodiment 108

A method of detecting an analyte, comprising:
(i) incubating a sample suspected of having an analyte of interest with a binding agent specific to the analyte under conditions that permit binding between the analyte and the binding agent; wherein the binding agent is associated with a nanoparticle or liposome comprising a signaling agent; wherein the signaling agent is not an enzyme; and wherein if the signaling agent is a pre-chemiluminophore, the nanoparticle is not crystalline.
(ii) dissociating the nanoparticle or liposome bound to the analyte, if any, to release the signaling agent such that it results in a signal change; and
(iii) determining presence or quantity of the analyte in the sample based on the signal change,
wherein
   the nanoparticle is a nanoparticle according to any one of embodiments 1-70, 76, 78-81, 88-105, or 17, or is a nanoparticle prepared according to embodiment 77; and
   the liposome is a liposome according to any one of embodiments 82-87.

### Embodiment 109

A method of detecting an analyte, comprising:
(i) incubating a sample suspected of having an analyte of interest with a binding agent specific to the analyte under conditions that permit binding between the analyte and the binding agent; wherein the binding agent is associated with a nanoparticle or liposome comprising a signaling agent; wherein the signaling agent is not an enzyme; and further wherein the binding agent is associated with the nanoparticle or liposome via an interaction other than an electrostatic interaction.
(ii) dissociating the nanoparticle or liposome bound to the analyte, if any, to release the signaling agent such that it results in a signal change; and
(iii) determining presence or quantity of the analyte in the sample based on the signal change,
wherein
   the nanoparticle is a nanoparticle according to any one of embodiments 1-70, 76, 78-81, 88-105, or 17, or is a nanoparticle prepared according to embodiment 77; and
   the liposome is a liposome according to any one of embodiments 82-87.

### Embodiment 110

The method of any one of embodiments 108 and 109, wherein the binding agent is selected from antibodies or antigen-binding fragments thereof, enzymes, oligonucleotides, DNA, RNA, PNA, or LNA, proteins, peptides, polypeptides, receptors, ligands, small molecules, aptamers, polysaccharides, plastibodies, affibodies, camelids, fibronectins, or a combination thereof.

### Embodiment 111

The method of embodiment 110, wherein the binding agent is an antibody or antigen-binding fragment thereof.

### Embodiment 112

The method of embodiment 111, wherein the antibody or antigen-binding fragment thereof is a primary antibody or a secondary antibody.

### Embodiment 113

The method of embodiment 110, wherein the binding agent is a small molecule.

### Embodiment 114

The method of any one of embodiments 108-113, wherein the binding agent is associated with the nanoparticle or liposome via covalent conjugation, non-covalent interaction, and/or adsorption.

### Embodiment 115

The method of embodiment 114, wherein the binding agent is associated with the nanoparticle or liposome via covalent conjugation.

### Embodiment 116

The method of any one of embodiments 108-115, wherein the dissociating step comprises treating the nanoparticle or liposome with a physical trigger, a chemical trigger, or a combination thereof.

### Embodiment 117

The method of embodiment 116, wherein the physical trigger is selected from the group consisting of thermal energy, electromagnetic energy, and/or sound energy.

### Embodiment 118

The method of embodiment 116, wherein the chemical trigger is an enzyme, a catalyst, a solvent, or an acid or base or other chemical agent, or a combination thereof.

### Embodiment 119

The method of any one of embodiments 108-118, wherein step (ii) and step (iii) are performed simultaneously in a solution.

### Embodiment 120

The method of embodiment 119, wherein the solution further comprises a chemical trigger for dissociating the nanoparticle or liposome.

### Embodiment 121

The method of embodiment 119 or 120, wherein the solution further comprises a pH modulator, a solvent, a catalyst, a co-catalyst, or a combination thereof.

### Embodiment 122

The method of any one of embodiments 108-121, wherein the sample is a biological sample.

### Embodiment 123

The method of embodiment 122, wherein the biological sample is selected from cells, cell lysate, FFPE (FASP Protein Digestion) digests, tissues including tissue biopsies or autopsy samples, whole blood, plasma, serum, urine, stool, saliva, cerebrospinal fluid, cord blood, chorionic villus samples amniotic fluid, and transcervical lavage fluid.

### Embodiment 124

A kit for detecting an analyte, comprising
(i) a binding agent specific to the analyte, wherein the binding agent is associated with a nanoparticle or liposome comprising a signaling agent; wherein the signaling agent is not an enzyme; and wherein if the signaling agent is a pre-chemiluminophore, the nanoparticle is not crystalline; and
(ii) a solution comprising reagents for performing a reaction that results in a signal change, once the signaling agent is released from the nanoparticle or liposome,
wherein
   the nanoparticle is a nanoparticle according to any one of embodiments 1-70, 76, 78-81, 88-105, or 17, or is a nanoparticle prepared according to embodiment 77; and
   the liposome is a liposome according to any one of embodiments 82-87.

### Embodiment 125

A kit for detecting an analyte, comprising
(i) a binding agent specific to the analyte, wherein the binding agent is associated with a nanoparticle or liposome comprising a signaling agent; wherein the signaling agent is not an enzyme; and further wherein the binding agent is associated with the nanoparticle via an interaction other than an electrostatic interaction; and
(ii) a solution comprising reagents for performing a reaction that results in a signal change, once the signaling agent is released from the nanoparticle or liposome,
wherein
   the nanoparticle is a nanoparticle according to any one of embodiments 1-70, 76, 78-81, 88-105, or 17, or is a nanoparticle prepared according to embodiment 77; and
   the liposome is a liposome according to any one of embodiments 82-87.

### Embodiment 126

A kit for detecting an analyte, comprising
(i) a nanoparticle or liposome comprising a signaling agent and one or more functional groups for associating the nanoparticle or liposome to a binding agent specific for an analyte; and
(ii) a solution comprising reagents for performing a reaction that results in a signal change, once the signaling agent is released from the nanoparticle or liposome,
wherein
   the nanoparticle is a nanoparticle according to any one of embodiments 1-70, 76, 78-81, 88-105, or 17, or is a nanoparticle prepared according to embodiment 77; and
   the liposome is a liposome according to any one of embodiments 82-87.

### Embodiment 127

The kit of embodiment 126, wherein the signaling agent is not an enzyme.

### Embodiment 128

The kit of any one of embodiments 124-127, wherein if the signaling agent is a pre-chemiluminophore, the nanoparticle is not crystalline.

### Embodiment 129

The kit of any one of embodiments 126-128, wherein the one or more functional groups are designed for covalent conjugation, non-covalent interaction, and/or adsorption.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A nanoparticle comprising
(a) a transition-metal catalyst; and
(b) one or more matrix-forming agents providing a dissociable matrix, wherein
the transition-metal catalyst is embedded in the matrix;
wherein
said transition-metal catalyst of (a) comprises a structure according to formula I, 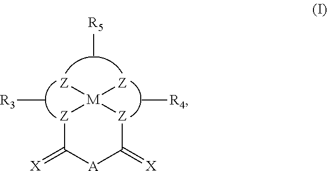
or an oxidized or reduced form thereof, wherein
M is a metal;
A is —CR₁R₂— or —NR_{1′}—;
wherein when A is —CR₁R₂—, R₁ and R₂ are the same or different, linked or nonlinked, and each is independently selected from the group consisting of substituents which are unreactive, form strong bonds intramolecularly within said R₁ and R₂ and with the carbon C to which they are bound, are sterically hindered and are conformationally hindered such that oxidative degradation of a metal complex of the compound is restricted when the complex is in the presence of an oxidizing medium; and
wherein when A is —NR_{1′}—, R_{1′} is C₁₋₂₀ alkyl, C₂₋₂₀ alkenyl, C₂₋₂₀ alkynyl, or phenyl;
Z is a metal complexing atom selected from the group consisting of N and 0;
X is a functionality;
wherein both Z and X are resistant to oxidative degradation such that each confers resistance to oxidative degradation to the metal complex of the compound when the complex is in the presence of an oxidizing medium;
R₃ is a unit joining the adjacent Z atoms selected from the group consisting of: 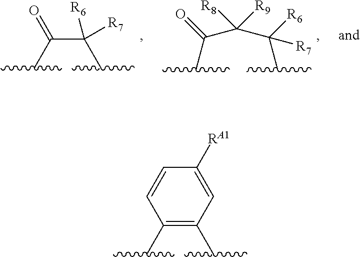
wherein R₆, R₇, R₈ and R₉ pairwise and cumulatively are the same or different and each is selected from the group consisting of hydrogen, C₁₋₂₀ alkyl, C₂₋₂₀ alkenyl, C₂₋₂₀ alkynyl, C₆₋₁₀ aryl, and halogen; or any pair of R₆, R₇, R₈ and R₉ can, together with the atoms to which they are attached, form a C₄₋₁₀ cycloalkyl;
R^{A1} is hydrogen, halogen, or —X¹—Y¹—Z¹, wherein
X¹ is —C(R^{X1})₂—, —C(O)—, —C(O)O—, —C(O)NH—, —CR^{X1}═CR^{X1}—, —NR^{X1}—, NR^{X1}C(O)—, —O—, or —OC(O)—, wherein R^{X1} is hydrogen or C₁₋₆ alkyl;
Y¹ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y¹ are optionally and independently replaced by -Cy¹-,
—NR^{Y1}—, —N(R^{Y1})C(O)—, —C(O)N(R^{Y1})—, —O—, —C(O)—, —OC(O)—, —C(O)O—, or —N═N—, wherein R^{Y1} is hydrogen or C₁₋₆ alkyl; and
each Cy¹ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
Z¹ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
R₄ is a unit joining the adjacent Z atoms comprised of 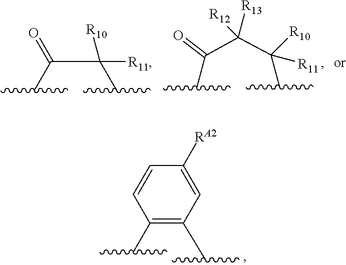
wherein R₁₀, R₁₁, R₁₂ and R₁₃ pairwise and cumulatively are the same or different and each is selected from the group consisting of hydrogen, C₁₋₂₀ alkyl, C₂₋₂₀ alkenyl, C₂₋₂₀ alkynyl, C₆₋₁₀ aryl, and halogen; or any pair of R₁₀, R₁₁, R₁₂ and R₁₃ can, together with the atoms to which they are attached, form a C₄₋₁₀ cycloalkyl;
R^{A2} is hydrogen, halogen, or —X²—Y²—Z², wherein
X² is —C(R^{X2})₂—, —C(O)—, —C(O)O—, —C(O)NH—, —CR^{X2}═CR^{X2}—, —NR^{X2}, —NR^{X2}C(O)—, —O—, or —OC(O)—, wherein R^{X2} is hydrogen or C₁₋₆ alkyl;
Y² is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y² are optionally and independently replaced by -Cy²-,
—NR^{Y2}—, —N(R^{Y2})C(O)—, —C(O)N(R^{Y2})—, —O—, —C(O)—, —OC(O)—, —C(O)O—, or —N═N—, wherein R^{Y2} is hydrogen or C₁₋₆ alkyl; and
each Cy² is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
Z² is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl
R₅ is a unit joining adjacent Z atoms selected from the group consisting of
(i) 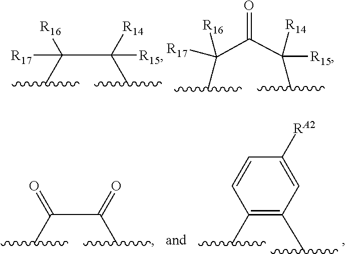
wherein R₁₄, R₁₅, R₁₆ and R₁₇ are the same or different and each is hydrogen, C₁₋₂₀ alkyl, C₂₋₂₀ alkenyl, C₂₋₂₀ alkynyl, C₆₋₁₀ aryl, and halogen;
or any pair of R₁₄, R₁₅, R₁₆ and R₁₇ can, together with the atoms to which they are attached, form a C₄₋₁₀ cycloalkyl;
R^{A3} is hydrogen, halogen, or —X³—Y³—Z³, wherein
X³ is —C(R^{X3})₂—, —C(O)—, —C(O)O—, —C(O)NH—, —CR^{X3}═CR^{X3}—, —NR^{X3}—, —NR^{X3}C(O)—, —O—, or —OC(O)—, wherein R^{X3} is hydrogen or C₁₋₆ alkyl;
Y³ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y³ are optionally and independently replaced by -Cy³-,
—NR^{Y3}—, —N(R^{Y3})C(O)—, —C(O)N(R^{Y3})—, —O—, —C(O)—, —OC(O)—, —C(O)O—, or —N═N—, wherein R^{Y3} is hydrogen or C₁₋₆ alkyl; and
each Cy³ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
Z³ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
optionally a counter ion selected from H₂O, ammonium, and halogen.

2. The nanoparticle of claim 1, wherein each Z is N.

3. The nanoparticle of claim 1, wherein each of R₁ and R₂ is selected, independently, from the group consisting of hydrogen, halogen, and C₁₋₂₀ alkyl, or wherein R₁ and R₂ link to form a C₃₋₁₀ cycloaliphatic group.

4. The nanoparticle of claim 1, wherein R₃ is a unit joining the adjacent Z atoms comprised of 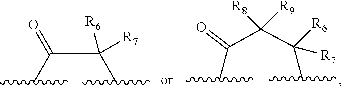
wherein each of R₆, R₇, R₈ and R₉ is, independently halogen, C₁₋₂₀ alkyl, C₂₋₂₀ alkenyl, or C₂₋₂₀ alkynyl; or
R₆ and R₇, or R₈ and R₉, link to form a C₃₋₁₀ cycloaliphatic group.

5. The nanoparticle of claim 1, wherein R₄ is a unit joining the adjacent Z atoms comprised of 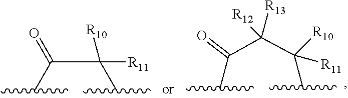
wherein each of R₁₀, R₁₁, R₁₂ and R₁₃ is, independently, halogen, C₁₋₂₀ alkyl, C₂₋₂₀ alkenyl, or C₂₋₂₀ alkynyl; or
R₁₀ and R₁₁, or R₁₂ and R₁₃, link to form a C₃₋₁₀ cycloaliphatic group.

6. The nanoparticle of claim 1, wherein R₅ is a unit joining adjacent Z atoms selected from the group consisting of 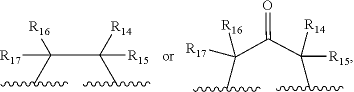
wherein each of R₁₄, R₁₅, R₁₆ and R₁₇ is independently selected from C₁₋₂₀ alkyl, C₆₋₁₂ aryl, and halogen, or R₁₄ and R₁₅, or R₁₆ and R₁₇, link to form a C₃₋₁₀ cycloaliphatic group.

7. The nanoparticle of claim 1, wherein R₅ is an optionally-substituted aryl or heteroaryl group.

8. The nanoparticle of claim 1, wherein one of any one of R₃, R₄, and R₅ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

9. The nanoparticle of any one of claims 1 to 8, wherein A is —CR₁R₂.

10. The nanoparticle of any one of claims 1 to 8, wherein A is —NR_{1′}—.

11. The nanoparticle of any one of claims 1 to 10, wherein the embedding of the catalyst in the matrix is not primarily governed by electrostatic interactions;

12. A nanoparticle comprising
(a) a transition-metal catalyst; and
(b) one or more matrix-forming agents providing a dissociable matrix, wherein the transition-metal catalyst is embedded in the matrix;
wherein
said transition-metal catalyst of (a) comprises a structure according to formula II, 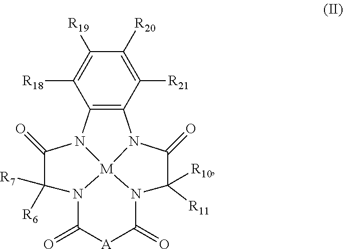
or an oxidized or reduced form thereof, wherein
M is a metal;
A is —CR₁R₂— or —NR_{1′}—;
wherein when A is —CR₁R₂—, each of R₁ and R₂ is, independently, hydrogen, C₁₋₂₀ alkyl, C₂₋₂₀ alkenyl, C₂₋₂₀ alkynyl, C₆₋₁₄ aryl, or halogen, or R₁ and R₂ may form, together with the carbon atom to which both are bound, a 3-6 membered ring; and
wherein when A is —NR_{1′}—, R_{1′} is C₁₋₂₀ alkyl, C₂₋₂₀ alkenyl, C₂₋₂₀ alkynyl, or phenyl;
each of R₆, R₇, R₁₀, and R₁₁ is, independently, hydrogen, C₁₋₂₀ alkyl, C₂₋₂₀ alkenyl, C₂₋₂₀ alkynyl, C₆₋₁₄ aryl, or halogen, or R₁ and R₂, or R₃ and R₄, or R₅ and R₆ may form, together with the carbon atom to which both are bound, a 3-10 membered ring; and
each of is R₁₈, R₁₉, R₂₀, and R₂₁ is, independently, halogen, hydrogen, C₁₋₂₀ alkyl, C₂₋₂₀ alkenyl, C₂₋₂₀ alkynyl, C₆₋₁₄ aryl, amino, nitro, azido, cyano, —OH, C₁₋₂₀ alkoxy, —SH, C₁₋₂₀ thioalkoxy, C₆₋₁₄ aryloxy, —CO₂H, a carboxylic ester, an N-hydrosuccinimide ester group, an isothiocyanate group, an isocyanide group, or a 5-10-membered heterocyclic group.

13. The nanoparticle of claim 12, wherein said transition-metal catalyst has a structure according to formula IIA,

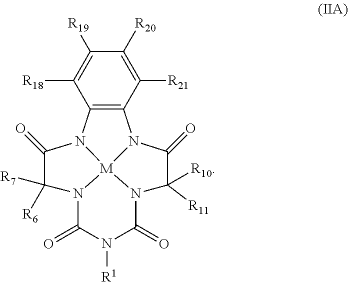

14. The nanoparticle of claim 12, wherein said transition-metal catalyst has a structure according to formula IIB,

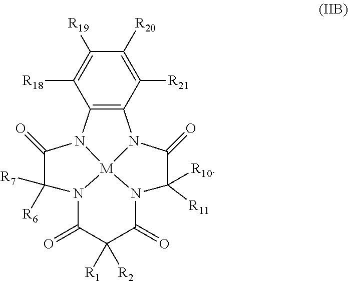

15. The nanoparticle of any one of claims 12-14, wherein each of R₁ and R₂ is selected, independently, from the group consisting of hydrogen, halogen, and C₁₋₂₀ alkyl, or wherein R₁ and R₂ link to form a C₃₋₁₀ cycloaliphatic group.

16. The nanoparticle of any one of claims 12-15, wherein one or more of R₆, R₇, R₁₀, and R₁₁ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

17. The nanoparticle of any one of claims 12-15, wherein one or more of R₁₈, R₁₉, R₂₀, and R₂₁ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

18. The nanoparticle of claim 12, comprising a transition metal catalyst having a structure that is 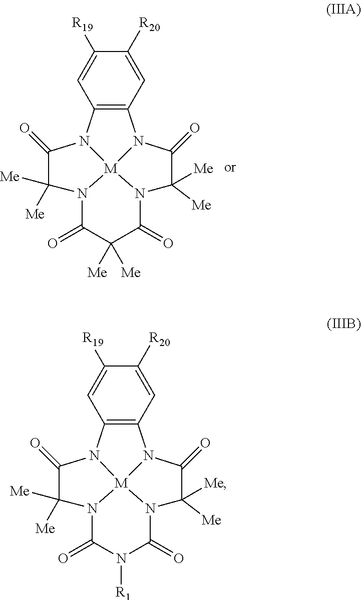
or an oxidized or reduced form thereof.

19. The nanoparticle of claim 18, wherein one or both of R₁₉ and R₂₀ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

20. The nanoparticle of claim 18, wherein the transition metal catalyst has a structure according to formula (IIIB) and R¹ optionally comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

21. The nanoparticle of claim 12, comprising a transition-metal catalyst having a structure that is, 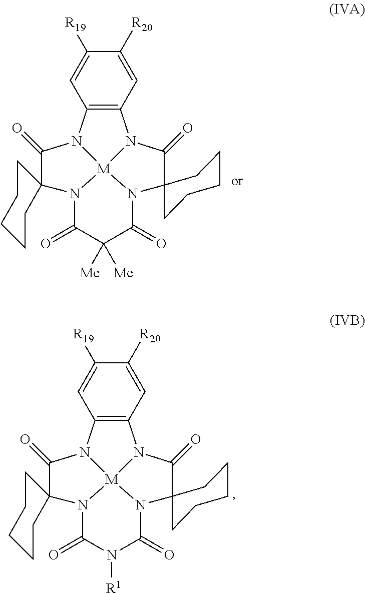
or an oxidized or reduced form thereof.

22. The nanoparticle of claim 21, wherein one or both of R₁₉ and R₂₀ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

23. The nanoparticle of claim 22, wherein one or both of R₁₉ and R₂₀ is a norbornene or cyclooctene.

24. The nanoparticle of claim 21, wherein the transition metal catalyst has a structure according to formula (IVB) and R¹ optionally comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

25. The nanoparticle of any one of claims 1-24, wherein M is a group 6, 7, 8, 9, 10, or 11 metal.

26. The nanoparticle of claim 25, wherein M is Cr, Mn, Fe, Co, Ni, or Cu.

27. The nanoparticle of any one of claims 12 to 26, wherein the embedding of the catalyst in the matrix is not primarily governed by electrostatic interactions;

28. A nanoparticle comprising
(a) a transition-metal catalyst; and
(b) one or more matrix-forming agents providing a dissociable matrix, wherein the transition-metal catalyst is embedded in the matrix;
wherein
said transition-metal catalyst of (a) comprises a structure according to formula V, 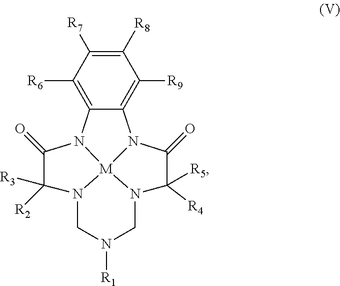
or an oxidized or reduced form thereof, wherein
M is a metal selected from the group consisting of Cr, Mn, Fe, Cu, Ni and Co;
R₁ is C₁₋₂₀ alkyl, C₂₋₂₀ alkenyl, C₂₋₂₀ alkynyl, or phenyl;
each of R₂, R₃, R₄, and R₅ is, independently, hydrogen, C₁₋₂₀ alkyl, C₂₋₂₀ alkenyl, C₂₋₂₀ alkynyl, or phenyl, or R₂ and R₃, or R₄ and R₅, combine to form a C₃₋₁₀ cycloaliphatic;
each of R₆, R₇, R₈, and R₉ is, independently, amino, nitro, azido, cyano, hydrogen, halogen, —NO₂, —COOH, —COOR₁₀, —COCl, —CN, C₁₋₂₀ alkyl, C₂₋₂₀ alkenyl, or C₂₋₂₀ alkynyl, wherein at least one of R₆, R₇, R₈, and R₉ is halogen, —NO₂, —COOH, —COOR₁₀, —COCl, or —CN;
R₁₀ is C₁₋₂₀ alkyl, C₂₋₂₀ alkenyl, C₂₋₂₀ alkynyl, phenyl, or 5-to-10-membered heterocyclyl.

29. The nanoparticle of claim 28, wherein one or both of R₇ and R₈ is halogen, —NO₂, —COOH, —COOR₁₀, —COCl, —CN, or a N-hydroxysuccinimide ester group.

30. The nanoparticle of claim 28, wherein R₁, R₇, and/or R₈ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

31. The nanoparticle of claim 28, wherein one or more of R₂, R₃, R₄, and R₅ comprises an amino group, an azido group, a thiol group, an alkenyl group, an alkynyl group, a carboxylic acid group, a carboxylic ester group, a N-hydroxysuccinimide ester group, an isothiocyanate group, an isocyanide group, a maleimide, an aldehyde, a norbornyl, a cyclooctenyl, or a tetrazine group.

32. The nanoparticle of claim 28, wherein each of R₂, R₃, R₄, and R₅ is C₁ alkyl.

33. The nanoparticle of any one of claims 1-32, wherein M is Fe(III) and the transition-metal catalyst further comprises a cation having a charge of +1.

34. The nanoparticle of any one of claims 1-33, wherein the transition-metal catalyst can mediate an oxidative or reductive transformation on a compound.

35. The nanoparticle of claim 34, wherein the transition-metal catalyst can mediate an oxidative reaction on a compound.

36. The nanoparticle of claim 35, wherein said compound is selected from: hydroethidine (HE); 1,3-diphenylisobenzofuran (DPBF), 2-(2-pyridyl)-benzothioazoline; 2,7-dichlorodihydrofluorescein (DCFH); 7-hydroxy-6-methoxy coumarin (scopoletin); N-acetyl-3,7-dihydroxyphenoxazine (Amplex Red); 4-hydroxy-3-methoxy-phenylacetic acid (HVA or homovanillic acid); dihydrorhodamine 123 (DHR); 4-(9-anthroyloxy)-2,2,6,6,-tetramethylpiperidine-1-oxyl; 1,3-cyclohexanedione (CHD); sodium terephthalate; coumarin-3-carboxylic acid (3-CCA); N-succinimidyl ester of coumarin-3-carboxylic acid (SECCA); 2-[6-(4′-hydroxy)phenoxy-3H-xanthen-3-on-9-yl]benzoic acid (HPF); 2-[6-(4′-amino)phenoxy-3H-xanthen-3-on-9-yl]benzoic acid (APF); cis-parinaric acid (cis-PnA, (18:14):9,11,13,15-cis-trans-trans-cis-octadecaenoic acid); 4,4-difluoro-5-(4-phenyl-1,3-butadienyl)-4-bora-3a,4a-diaza-s-indacene-3-undecanoic acid (C₁₁—BODIPY); lipophilic fluorescein derivatives; dipyridamole; diphenyl-1-pyrenylphosphine (DPPP); 2,7-dichlorodihydrofluorescein acetate (DCFH-DA); beta-physcoerythrin; fluorescein; and 6-carboxyfluorescein, or a derivative thereof.

37. A nanoparticle comprising
(a) a transition-metal catalyst having the structure M(X)ₙ(R)ₒ, wherein
M is a transition-metal;
n is 0, 1, 2, 3, or 4;
o is 2, 3, 4, 5, or 6:
X is an ion of a Group V, VI, or VII element;
R is a ligand selected from monodentate phosphine ligands, bidentate phosphine ligands, monodentate Schiff base ligands, bidentate Schiff base ligands, tridentate Schiff base ligands, macrocyclic ligands, pentamethylcyclopentadiene, monodentate arsine, or N-heterocyclic carbene ligands; and
(b) one or more matrix-forming agents providing a dissociable matrix, wherein the transition-metal catalyst is embedded in the matrix;
wherein
said transition-metal catalyst of (a) can catalyze a bond formation reaction or a bond cleavage reaction that modulates the fluorescent or chromogenic properties of a substrate compound.

38. The nanoparticle of any one of claims 1-37, wherein at least one of said one or more matrix-forming agents comprises organic polymers, waxes, fats, oils, surfactants, or a combination thereof.

39. The nanoparticle of any one of claims 1-38, wherein the matrix comprises a covalent bond to the transition-metal catalyst.

40. The nanoparticle of any one of claims 1-39, wherein the nanoparticle comprises an outer surface that comprises one or more functional groups for conjugating the nanoparticle to a binding agent.

41. The nanoparticle of claim 40, wherein the binding agent comprises an antibody, ligand, protein, small molecule, aptamer, ss-DNA, ss-RNA, or ss-PNA.

42. A nanoparticle comprising
(a) a transition-metal catalyst; and
(b) one or more matrix-forming agents providing a dissociable matrix, wherein the transition-metal catalyst is embedded in the matrix;
wherein
said transition-metal catalyst of (a) is selected from: 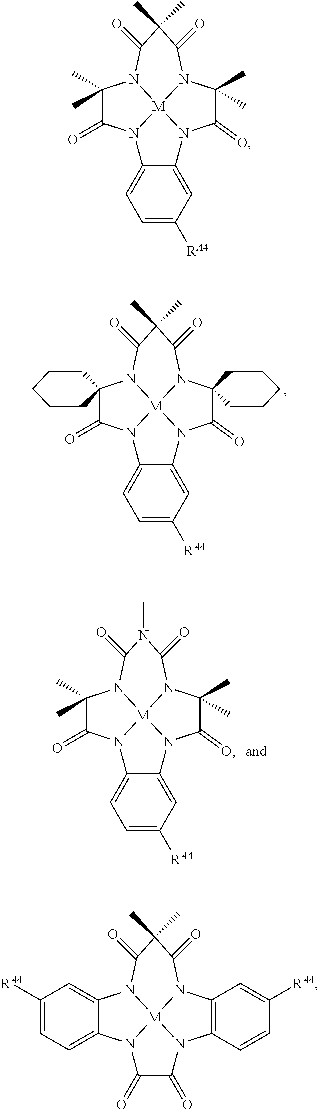
wherein
M is a metal selected from Fe, Mg, Cu, Mn, Pd, Pt, Ag, Ru, and Ce; and
R^{A4} is hydrogen, halogen, or —X⁴—Y⁴—Z⁴, wherein
X⁴ is —C(R^{X4})₂—, —C(O)—, —C(O)O—, —C(O)NH—, —CR^{X4}═CR^{X4}—, —NR^{X4}—, —NR^{X4}C(O)—, —O—, or —OC(O)—, wherein R^{X4} is hydrogen or C₁₋₆ alkyl;
Y⁴ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y⁴ are optionally and independently replaced by -Cy⁴-,
—NR^{Y4}—, —N(R^{Y4})C(O)—, —C(O)N(R^{Y4})—, —O—, —C(O)—, —OC(O)—, —C(O)O—, or —N═N—, wherein R^{Y4} is hydrogen or C₁₋₆ alkyl; and
each Cy⁴ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
Z⁴ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
a suitable counter ion selected from H₂O and halogen.

43. A liposome comprising a signal-inducing agent that is a transition metal catalyst having a structure according to formula II, formula IIA, formula IIB, formula IIIA, formula IIIB, formula IVA, formula IVB, formula V, or an oxidized or reduced form thereof.

44. A liposome comprising a signal-inducing agent that is a transition metal catalyst having a structure selected from 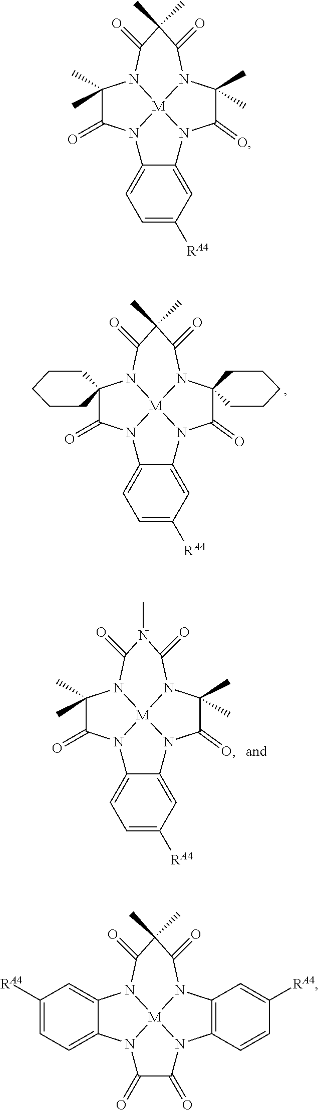
wherein
M is a metal selected from Fe, Mg, Cu, Mn, Pd, Pt, Ag, Ru, and Ce; and
R^{A4} is hydrogen, halogen, or —X⁴—Y⁴—Z⁴, wherein
X⁴ is —C(R^{X4})₂—, —C(O)—, —C(O)O—, —C(O)NH—, —CR^{X4}═CR^{X4}—, —NR^{X4}, —NR^{X4}C(O)—, —O—, or —OC(O)—, wherein R^{X4} is hydrogen or C₁₋₆ alkyl;
Y⁴ is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y⁴ are optionally and independently replaced by -Cy⁴-,
—NR^{Y4}—, —N(R^{Y4})C(O)—, —C(O)N(R^{Y4})—, —O—, —C(O)—, —OC(O)—, —C(O)O—, or —N═N—, wherein R^{Y4} is hydrogen or C₁₋₆ alkyl; and
each Cy⁴ is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and
Z⁴ is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl; and
a suitable counter ion selected from H₂O and halogen.

45. A nanoparticle comprising a polymeric matrix, wherein said polymeric matrix comprises a polymer that comprises a repeating unit comprising one or more signal-inducing agents.

46. The nanoparticle of claim 45, wherein said polymer has a structure according to the following formula, 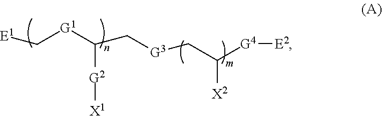
E¹ is independently hydrogen;
E² is independently hydrogen or a detection species;
each of G¹, G², G³, and G⁴ is independently a covalent bond or cleavable group;
n is independently an integer of 1 to 100;
m is independently an integer of 0 to 100;
X¹ is a signal-inducing agent; and
X² is hydrogen or non-payload element for stability.

47. The nanoparticle of claim 45, wherein said polymer comprises a repeating unit having a structure according to substructure S3.13, 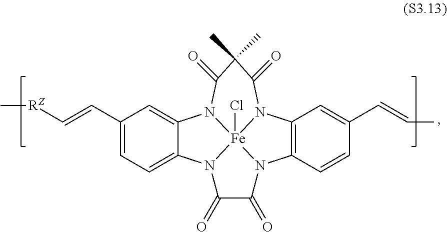
wherein R^{Z} is hydrogen, halogen, or —X^{Z1}—Y^{Z1}—Z^{Z1}, wherein X^{Z1} is —C(R^{XZ1})₂—, —C(O)—, —C(O)O—, —C(O)NH—, —CR^{XZ1}═CR^{XZ1}—, —NR^{XZ1}—, —NR^{XZ1}C(O)—, —O—, or —OC(O)—, wherein R^{XZ1} is hydrogen or C₁₋₆ alkyl; Y^{Z1} is a covalent bond, a bivalent linker comprising two or more repeating units of ethylene glycol, or an optionally substituted, bivalent C₁₋₂₀ saturated or unsaturated, straight or branched, hydrocarbon chain, wherein one, two, or three methylene units of Y^{Z1} are optionally and independently replaced by -Cy^{Z11}-, —NR^{YZ1}—, —N(R^{YZ1})C(O)—, —C(O)N(R^{YZ1})—, —O—, —C(O)—, —OC(O)—, —C(O)O—, or —N═N—, wherein R^{YZ1} is hydrogen or C₁₋₆ alkyl; and each Cy^{Z1} is independently an optionally substituted bivalent ring selected from C₆₋₁₀ arylene, a C₃₋₁₀ cycloalkylene, a 3 to 7 membered heterocyclylene having 1-2 heteroatoms independently selected from nitrogen, oxygen, or sulfur, or a 5-6 membered heteroarylene having 1-3 heteroatoms independently selected from nitrogen, oxygen, or sulfur; and Z^{Z1} is hydrogen or a functional group selected from an optionally substituted C₂₋₈ unsaturated hydrocarbon chain, wherein one or two methylene units are optionally and independently replaced by C(O), trans-cyclooctenyl, thiolyl, and tetrazinyl.

48. A nanoparticle comprising
(a) a compound that is chemiluminophore, a chemiluminophore precursor, a soluble absorber, or a soluble absorber precursor; and
(b) optionally one or more matrix-forming agents providing a matrix, wherein the compound of (a) is embedded in the matrix;
wherein
the embedding is not primarily governed by electrostatic interactions and/or the embedding is primarily governed by surfactant stabilization during formation of the matrix,
the matrix sequesters the compound of (a) until said matrix is dissociated, and
the nanoparticle comprises at least about 20 mol % of the compound of (a).

49. The nanoparticle of claim 48, wherein the matrix sequesters the compound of (a) until said matrix is dissociated.

50. The nanoparticle of claim 48 or 49, wherein the compound of (a) is an acylated fluorescein or an acylated rhodamine.
